(19) United States
(12) Patent Application Publication
(10) Pub. No.: US 2005/0171079 A1
(43) Pub. Date: Aug. 4, 2005

(54) Amino-substituted tricyclic derivatives and methods of use

(76) Inventors: Michael R. Schrimpf, Grayslake, IL (US); Kevin B. Sippy, Antioch, IL (US); Jianguo Ji, Libertyville, IL (US); Tao Li, Grayslake, IL (US); Jennifer M. Pace, Grayslake, IL (US); Clark A. Briggs, Libertyville, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/772,192

(22) Filed: Feb. 4, 2004

(51) Int. Cl. A61K 31/397; A61K 31/551; A61K 31/4545; A61K 31/4025

(52) U.S. Cl. 514/210.01; 514/221; 514/249; 514/412; 514/408; 514/649; 548/950; 548/528; 514/319; 546/203; 546/135; 540/575; 514/316; 514/422; 548/517

(57) ABSTRACT

Compounds of formula (I) 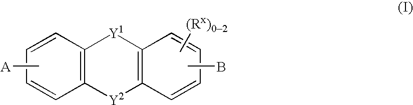
wherein A and B are amine-substituted sidechains, Y¹ and Y² form various tricyclic cores, and R^{x} is an optional substituent. Compounds and compositions of formula (I) are contemplated as well as methods for treating conditions or disorders prevented by or ameliorated by α7 nAChR ligands that encompass compounds of formula (I) and other tricyclic derivatives.

### BACKGROUND OF THE INVENTION

1. Technical Field

The invention relates to amine-substituted tricyclic derivatives, compositions comprising such compounds, and methods of treating conditions and disorders using such compounds and compositions.

2. Description of Related Technology

Nicotinic acetylcholine receptors (nAChRs) are widely distributed throughout the central (CNS) and peripheral (PNS) nervous systems. Such receptors play an important role in regulating CNS function, particularly by modulating release of a wide range of neurotransmitters, including, but not necessarily limited to acetylcholine, norepinephrine, dopamine, serotonin and GABA. Consequently, nicotinic receptors mediate a very wide range of physiological effects, and have been targeted for therapeutic treatment of disorders relating to cognitive function, learning and memory, neurodegeneration, pain and inflammation, psychosis and sensory gating, mood and emotion, among others.

Many subtypes of the nAChR exist in the CNS and periphery. Each subtype has a different effect on regulating the overall physiological function. Typically, nAChRs are ion channels that are constructed from a pentameric assembly of subunit proteins. At least 12 subunit proteins, α2-α10 and β2-β4, have been identified in neuronal tissue. These subunits provide for a great variety of homomeric and heteromeric combinations that account for the diverse receptor subtypes. For example, the predominant receptor that is responsible for high affinity binding of nicotine in brain tissue has composition (α4)₂(β2)₃ (the α4β2 subtype), while another major population of receptors is comprised of homomeric (α7)₅ (the (α7 subtype) receptors.

Certain compounds, like the plant alkaloid nicotine, interact with all subtypes of the nAChRs, accounting for the profound physiological effects of this compound. While nicotine has been demonstrated to have many beneficial properties, not all of the effects mediated by nicotine are desirable. For example, nicotine exerts gastrointestinal and cardiovascular side effects that interfere at therapeutic doses, and its addictive nature and acute toxicity are well-known. Ligands that are selective for interaction with only certain subtypes of the nAChR offer potential for achieving beneficial therapeutic effects with an improved margin for safety.

The α7 nAChRs have been shown to play a significant role in enhancing cognitive function, including aspects of learning, memory and attention (Levin, E. D., J. Neurobiol. 53: 633-640, 2002). For example, α7nAChRs have been linked to conditions and disorders related to attention deficit disorder, attention deficit hyperactivity disorder (ADHD), Alzheimer's disease (AD), mild cognitive impairment, senile dementia, dementia associated with Lewy bodies, dementia associated with Down's syndrome, AIDS dementia, Pick's Disease, as well as cognitive deficits associated with schizophrenia, among other systemic activities.

The activity at the α7 nAChRs can be modified or regulated by the administration of α7 nAChR ligands. The ligands can exhibit antagonist, agonist, or partial agonist properties. Thus, α7 ligands have potential in treatment of various cognitive disorders.

Although various classes of tricyclic compounds are known, it would be beneficial to provide additional compounds demonstrating activity at the α7 nAChRs that can be incorporated into pharmaceutical compositions useful for therapeutic methods. Specifically, it would be beneficial to provide tricyclic compounds that interact selectively with α7-containing neuronal nAChRs compared to other subtypes.

### SUMMARY OF THE INVENTION

The invention is directed to amine-substituted tricyclic derivative compounds as well as compositions comprising such compounds, and method of using the same. Compounds of the invention have the formula (I): 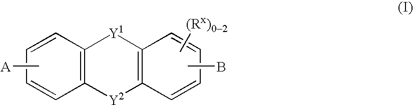
or a pharmaceutically acceptable salt, ester, amide, or prodrug thereof, wherein:
   A and B are each independently selected from the group consisting of hydrogen; halogen; alkoxy; amino; alkylamino; acylamino; dialkylamino; cyano; nitro; and —SO₃H; and
a group of formula (a): 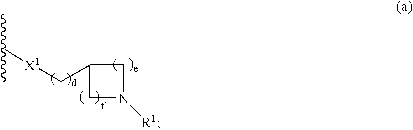
a group of formula (b): 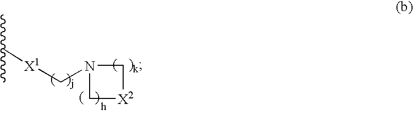
a group of formula (c): 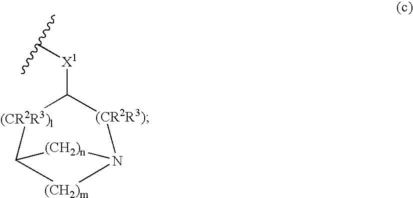
a group of formula (d): 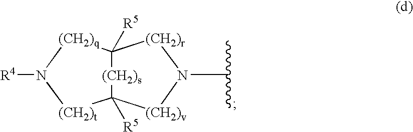
a group of formula (e): 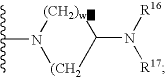
a group of formula (f): 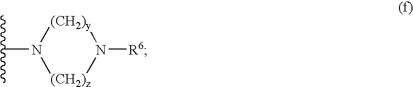
a group of formula (g): 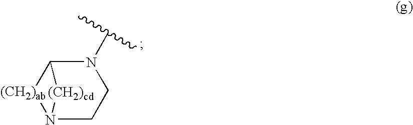
(h) —C≡CCH₂NR⁷R⁸; and (i) —O—(C(R²⁰)₂₋₃N(R²¹)(R²²); provided that at least one of A or B is a group selected from (a)-(i); with the proviso that if A or B is selected from group (a), (b), or (f) when y and z are both two, then A and B are different;
   X¹ at each occurrence is selected from the group consisting of O, S, and —N(R⁹)—;
   X² at each occurrence is selected from the group consisting of O, S, —CH₂—, and —N(R¹⁰)—;
   Y¹ is independently selected from the group consisting of —O(O)—, —CH₂—, —CH(OH)—, —C(S)—, —N(R¹¹)—, —O—, —S—, —S(O)—, —S(O)₂—, —C(O)NH—, and —S(O)₂NH—, provided that if Y¹ is —C(O)—, —O—, —S—, or —N(R¹¹)— and one of A or B is selected from a group (a), (b), or (f), then the other of A or B is selected from the group consisting of alkoxy, dialkylamino, cyano, and —SO₃H;
   Y² is a bond or Y² is independently selected from —O—, —S—, and —N(R¹²)—;
   R¹ is independently selected from hydrogen and alkyl;
   R² and R³ at each occurrence are each independently selected from the group consisting of hydrogen and alkyl;
   R⁴ and R⁶ at each occurrence are each independently selected from the group consisting of hydrogen and alkyl;
   R⁵ at each occurrence are each independently selected from the group consisting of hydrogen, alkyl, and alkoxycarbonyl;
   R⁷ and R⁸ are each independently selected from hydrogen and alkyl or R⁷ and R⁸ taken together with the nitrogen atom to which each is attached form a 4- to 8-membered cyclic amine;
   R⁹, R¹⁰, R¹¹, and R¹² at each occurrence are each independently selected from hydrogen and alkyl;
   R¹⁶ and R¹⁷ are each independently selected from hydrogen and alkyl, or R¹⁶ and R¹⁷ taken together with the nitrogen atom to which each is attached form a 4 to 8-membered cyclic amine;
   R²⁰ is selected from the group consisting of hydrogen and alkyl;
   R²¹ and R²² are each independently selected from the group consisting of hydrogen and alkyl;
   R^{x} is independently selected at each occurrence from the group consisting of hydrogen, halogen, alkoxy, amino, alkylamino, dialkylamino, acylamino, dialkylaminoalkyl, and cyano;
   d is independently selected from 0 or 1;
   e and f are each independently selected from 0, 1, 2 or a provided that the sum total of e and f is 2, 3, or 4, provided that when d is 0, e and f are selected from 1, 2 or 3;
   j is independently selected from 2 or 3;
   h and k are each independently selected from 0, 1, or 2, provided that the sum total of h and k is 2, 3, or 4, provided that when X² is O, S, or N(R¹⁰), h and k are both 2;
   l is 0 or 1, m is 2 or 3, and n is 0, 1, or 2, provided that the sum total of l, m, and n is 4, 5, or 6;
   q, r, s, t, and v are each independently selected from 0, 1, or 2, provided that the sum of q and r; t and v; q, s, and t; and r, s, and v; are each at least 1, and further provided that the sum total of q, r, s, t, and v is 2, 3, 4, or 5, provided that when the sum total is 5 and Y¹ is —O—, —S—, or —N(R¹¹)— and Y² is a bond, both A and B are other than hydrogen;
   w and x are each independently selected from 1, 2, or 3, provided that the sum total of w and x is 3, 4, 5, or 6;
   y and z are each independently selected from 2, 3, or 4, provided that the sum total of y and z is 4, 5, or 6; and
   ab is 2 or 3, and cd is 1 or 2.

Another aspect of the invention relates to pharmaceutical compositions comprising compounds of the invention. Such compositions can be administered in accordance with a method of the invention, typically as part of a therapeutic regimen for treatment or prevention of conditions and disorders related to nAChR activity, and more particularly α7 nAChR activity.

Yet another aspect of the invention relates to a method of selectively modulating to nAChR activity, for example α7 nAChR activity. The method is useful for treating and/or preventing conditions and disorders related to α7 nAChR activity modulation in mammals. More particularly, the method is useful for conditions and disorders related to attention deficit disorder, attention deficit hyperactivity disorder (ADHD), Alzheimer's disease (AD), mild cognitive impairment, senile dementia, AIDS dementia, Pick's Disease, dementia associated with Lewy bodies, dementia associated with Down's syndrome, amyotrophic lateral sclerosis, Huntington's disease, diminished CNS function associated with traumatic brain injury, acute pain, post-surgical pair) chronic pain, inflammatory pain, neuropathic pain, infertility, lack of circulation, need for new blood vessel growth associated with wound healing, more particularly circulation around a vascular occlusion, need for new blood vessel growth associated with vascularization of skin grafts, ischemia, inflammation, wound healing, and other complications associated with diabetes, among other systemic activities.

The compounds, compositions comprising the compounds, and methods for treating or preventing conditions and disorders by administering the compounds are further described herein.

### DETAILED DESCRIPTION OF THE INVENTION

Definition of Terms

Certain terms as used in the specification are intended to refer to the following definitions, as detailed below.

The term “acyl”, as used herein, means an alkyl group, as defined herein, appended to the parent molecular moiety through a carbonyl group, as defined herein. Representative examples of acyl include, but are not limited to, acetyl, 1-oxopropyl, 2,2-dimethyl-1-oxopropyl, 1-oxobutyl, and 1-oxopentyl.

The term “acyloxy”, as used herein, means an acyl group, as defined herein, appended to the parent molecular moiety through an oxygen atom. Representative examples of acyloxy include, but are not limited to, acetyloxy, propionyloxy, and isobutyryloxy.

The term “alkenyl”, as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy”, as used herein, means an alkyl group as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkoxy”, as used herein, means an alkoxy group, as defined herein, appended to the parent molecular moiety through another alkoxy group, as defined herein. Representative examples of alkoxyalkoxy include, but are not limited to, tert-butoxymethoxy, 2-ethoxyethoxy, 2-methoxyethoxy, and methoxymethoxy.

The term “alkoxyalkyl”, as used herein, means an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl”, as used herein, means an alkoxy group, as defined herein, appended to the parent molecular moiety through a carbonyl group, represented by —C(O)—, as defined herein. Representative examples of alkoxycarbonyl include, but are not limited to, methoxycarbonyl, ethoxycarbonyl, and tert-butoxycarbonyl.

The term “alkoxysulfonyl”, as used herein, means an alkoxy group, as defined herein, appended to the parent molecular moiety through a sulfonyl group, as defined herein. Representative examples of alkoxysulfonyl include, but are not limited to, methoxysulfonyl, ethoxysulfonyl and propoxysulfonyl.

The term “alkyl”, as used herein, means a straight or branched chain hydrocarbon containing from 1 to 6 carbon atoms. Representative examples of alkyl include, but are not limited to, methyl, ethyl, n-propyl, isopropyl, n-butyl, sec-butyl, iso-butyl, tert-butyl, n-pentyl, isopentyl, neopertyl, and n-hexyl.

The term “alkylcarbonyl”, as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonyloxy”, as used herein, means an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy.

The term “alkylsulfonyl”, as used herein, means an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylthio”, as used herein, means an alkyl group, as defined herein, appended to the parent molecular moiety through a sulfur atom. Representative examples of alkylthio include, but are not limited, methylthio, ethylthio, tert-butylthio, and hexylthio.

The term “alkynyl”, as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “amino”, as used herein, means —NH₂.

The term “acylamino”, as used herein, means an acyl group, as defined herein, appended to the parent molecular moiety through an amino group, as defined herein.

The term “alkylamino”, as used herein, means an alkyl group, as defined herein, appended to the parent molecular moiety through an amino group, as defined herein.

The term “dialkylamino”, as used herein, means two independently selected alkyl groups, as defined herein, appended to the parent molecular moiety through an amino group, as defined herein.

The term “dialkylaminoalkyl”, as used herein, means a dialkylamino, as defined herein, appended to the parent molecular moiety through an alkyl group, as defined herein.

The term “amido”, as used herein, means an amino, alkylamino, or dialkylamino group appended to the parent molecular moiety through a carbonyl group, as defined herein. Representative examples of amido include, but are not limited to, aminocarbonyl, methylaminocarbonyl, dimethylaminocarbonyl, and ethylmethylaminocarbonyl.

The term “aryl”, as used herein, means a monocyclic or bicyclic aromatic ring system. Representative examples of aryl include, but are not limited to, phenyl and naphthyl.

The aryl groups of this invention are substituted with 0, 1, 2, 3, 4, or 5 substituents independently selected from acyl, acyloxy, alkenyl, alkoxy, alkoxyalkoxy, alkoxyalkyl, alkoxycarbonyl, alkoxyimino, alkoxysulfonyl, alkyl, alkylsulfonyl, alkynyl, amino, carboxy, cyano, formyl, haloalkoxy, haloalkyl, halo, hydroxy, hydroxyalkyl, mercapto, nitro, thioalkoxy, —NR_{A}R_{B}, (NR_{A}R_{B})alkyl, (NR_{A}R_{B})alkoxy, (NR_{A}R_{B})carbonyl, and (NR_{A}R_{B})sulfonyl.

The term “carbonyl”, as used herein, means a —C(O)— group.

The term “carboxy”, as used herein, means a —CO₂H group.

The term “cyano”, as used herein, means a —CN group.

The term “cyclic amine”, as used herein, means a heterocycle group, as defined herein, wherein the heteroatom is nitrogen. Typically, cyclic amine groups are 4- to 6-membered rings containing one nitrogen atom.

The term “formyl”, as used herein, means a —C(O)H group.

The term “halo” or “halogen”, as used herein, means —Cl, —Br, —I or —F.

The term “haloalkoxy”, as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl”, as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3 fluoropentyl.

The term “heteroaryl” means an aromatic five- or six-membered ring containing 1, 2, 3, or 4 heteroatoms independently selected from nitrogen, oxygen, or sulfur. The heteroaryl groups are connected to the parent molecular moiety through a carbon or nitrogen atom. Representative examples of heteroaryl include, but are not limited to, furyl, imidazolyl, isoxazolyl, isothiazolyl, oxadiazolyl, oxazolyl, pyrazinyl, pyrazolyl, pyridazinyl, pyridinyl, pyrimidinyl, pyrrolyl, tetrazolyl, thiadiazolyl, thiazolyl, thienyl, triazinyl, and triazolyl.

The heteroaryl groups of the invention are substituted with 0, 1, 2, or 3 substituents independently selected from alkenyl, alkoxy, alkoxyalkoxy, alkoxyalkyl, alkoxycarbonyl, alkoxysulfonyl, alkyl, alkylcarbonyl, alkylcarbonyloxy, alkylsulfonyl, alkylthio, alkynyl, carboxy, cyano, formyl, haloalkoxy, haloalkyl, halo, hydroxy, hydroxyalkyl, mercapto, nitro, —NR_{A}R_{B}, (NR_{A}R_{B})alkyl, (NR_{A}R_{B})alkoxy, (NR_{A}R_{B})carbonyl, and (NR_{A}R_{B})sulfonyl.

The term “heterocycle,” as used herein, refers to a four, five, six, seven or eight membered ring containing one, two, or three heteroatoms independently selected from the group consisting of nitrogen, oxygen, and sulfur. The four membered ring has zero double bond and five membered ring has zero or one double bond. The six membered ring has zero, one, or two double bonds. The seven and eight membered rings have zero. one, two, or three double bonds. The term “heterocycle” also includes bicyclic groups in which the heterocycle ring is fused to a phenyl group, a monocyclic cycloalkenyl group, as defined herein, a monocyclic cycloalkyl group, as defined herein, or another monocyclic heterocycle group, as defined herein; and tricyclic groups in which a bicyclic system is fused to a phenyl group, a monocyclic cycloalkenyl group, as defined herein, a monocyclic cycloalkyl group, as defined herein, or another monocyclic heterocycle group. The heterocycle groups of the present invention can be attached to the parent molecular moiety through a carbon atom or a nitrogen atom. Representative examples of heterocycle include, but are not limited to, azetidinyl, azepanyl, azocanyl, morpholinyl, piperazinyl, piperidinyl, pyrrolidinyl, and thiomorpholinyl.

The heterocycles of the present invention are substituted with 0, 1, 2, 3, or 4 substituents independently selected from acyl, acyloxy, alkenyl, alkoxy, alkoxyalkoxy, alkoxyalkyl, alkoxycarbonyl, alkoxyimino, alkoxysulfonyl, alkyl, alkylsulfonyl, alkynyl, amido, arylalkyl, arylalkoxycarbonyl, carboxy, cyano, formyl, haloalkoxy, haloalkyl, halo, hydroxy, hydroxyalkyl, mercapto, nitro, oxo, thioalkoxy, —NR_{A}R_{B}, and (NR_{A}R_{B})sulfonyl.

The term “heterocyclealkyl”, as used herein, refers to a heterocycle group, as defined herein, appended to the parent molecular moiety through an alkyl group, as defined herein. Representative examples of heterocyclealkyl include, but are not limited to, 1-methylpyrrolidin-2-ylmethyl, azetidin-2-ylmethyl, 1-methylazetidin-2-ylmethyl, pyrrolidin-3-ylethyl, and 1-methylpyrrolidin-3-ylethyl.

The term “bicyclic heteroaryl” refers to fused aromatic nine- and ten-membered bicyclic rings containing 1, 2, 3, or 4 heteroatoms independently selected from nitrogen, oxygen, or sulfur, or a tautomer thereof. The bicyclic heteroaryl groups are connected to the parent molecular moiety through a carbon or nitrogen atom. Representative examples of bicyclic heteroaryl rings include, but are not limited to, indolyl, benzothiazolyl, benzofuranyl, isoquinolinyl, and quinolinyl. Bicyclic heteroaryl groups of the invention are substituted with 0, 1, 2, or 3 substituents independently selected from alkenyl, alkoxy, alkoxyalkoxy, alkoxyalkyl, alkoxycarbonyl, alkoxysulfonyl, alkyl, alkylcarbonyl, alkylcarbonyloxy, alkylsulfonyl, alkylthio, alkynyl, carboxy, cyano, formyl, haloalkoxy, haloalkyl, halo, hydroxy, hydroxyalkyl, mercapto, nitro, NR_{A}R_{B}, (NR_{A}R_{B})alkyl, (NR_{A}R_{B})alkoxy, (NR_{A}R_{B})carbonyl, and (NR_{A}R_{B})sulfonyl.

The term “hydroxy”, as used herein, means an —OH group.

The term “hydroxyalkyl”, as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “mercapto”, as used herein, means a —SH group.

The term “nitro”, as used herein, means a —NO₂ group.

The term “—NR_{A}R_{B}”, as used herein, means two groups, R_{A} and R_{B}, which are appended to the parent molecular moiety through a nitrogen atom. R_{A} and R_{B} are each independently hydrogen, alkyl, alkylcarbonyl, or formyl. Representative examples of —NR_{A}R_{B} include, but are not limited to, amino, methylamino, acetylamino, and acetylmethylamino.

The term “(NR_{A}R_{B})alkyl”, as used herein, means a —NR_{A}R_{B} group, as defined herein, appended to the parent molecular moiety through an alkyl group, as defined herein. Representative examples of (NR_{A}R_{B})alkyl include, but are not limited to, (amino)methyl, (dimethylamino)methyl, and (ethylamino)methyl.

The term “(NR_{A}R_{B})alkoxy”, as used herein, means a —NR_{A}R_{B} group, as defined herein, appended to the parent molecular moiety through an alkoxy group, as defined herein. Representative examples of (NR_{A}R_{B})alkoxy include, but are not limited to, (amino)methoxy, (dimethylamino)methoxy, and (diethylamino)ethoxy.

The term “(NR_{A}R_{B})carbonyl”, as used herein, means a —NR_{A}R_{B} group, as defined herein, appended to the parent molecular moiety through a carbonyl group, as defined herein. Representative examples of (NR_{A}R_{B})carbonyl include, but are not limited to, aminocarbonyl, (methylamino)carbonyl, (dimethylamino)carbonyl, and (ethylmethylamino)carbonyl.

The term “(NR_{A}R_{B})sulfonyl”, as used herein, means a —NR_{A}R_{B} group, as defined herein, appended to the parent molecular moiety through a sulfonyl group, as defined herein. Representative examples of (NR_{A}R_{B})sulfonyl include, but are not limited to, aminosulfonyl, (methylamino)sulfonyl, (dimethylamino)sulfonyl, and (ethylmethylamino)sulfonyl.

The term “sulfonyl”, as used herein, means a —S(O)₂— group.

The term “thioalkoxy”, as used herein, means an alkyl group, as defined herein, appended to the parent molecular moiety through a sulfur atom. Representative examples of thioalkoxy include, but are no limited to, methylthio, ethylthio, and propylthio.

Although typically it may be recognized that an asterisk is used to indicate that the exact subunit composition of a receptor is uncertain, for example α3b4* indicates a receptor that contains the α3 and β4 proteins in combination with other subunits, the term α7 as used herein is intended to include receptors wherein the exact subunit composition is both certain and uncertain. For example, as used herein α7 includes homomeric (α7)₅ receptors and α7* receptors, which denote a nAChR containing at least one α7 subunit.

Compounds of the Invention

Compounds of the invention can have the formula (I) as described above.

Preferred moieties for the group of formula (a) are azetidinyloxy, N-methylazetidinyloxy, pyrrolidinyloxy, N-methylpyrrolidinyloxy, piperidinyloxy, N-methylpiperidinyloxy; azetidinylmethoxy, N-methylazetidinylmethoxy, pyrrolidinylmethoxy, N-methylpyrrolidinylmethoxy, piperidinylmethoxy, N-methylpiperidinylmethoxy, and the like.

Specific examples of rings suitable for a group of formula (b) include, but are not limited to,

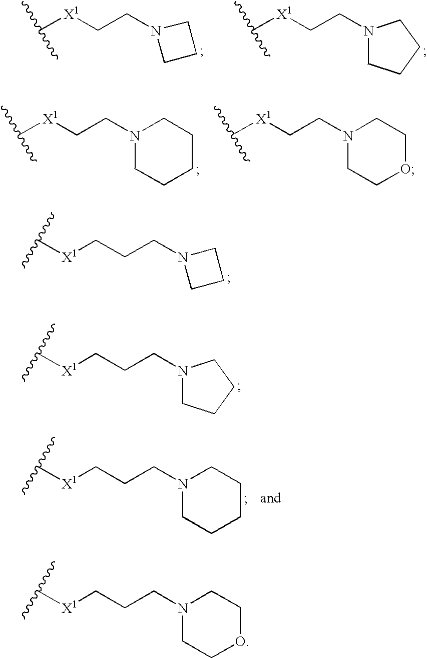

Specific examples of rings suitable for a group of formula (c) include, but are not limited to, 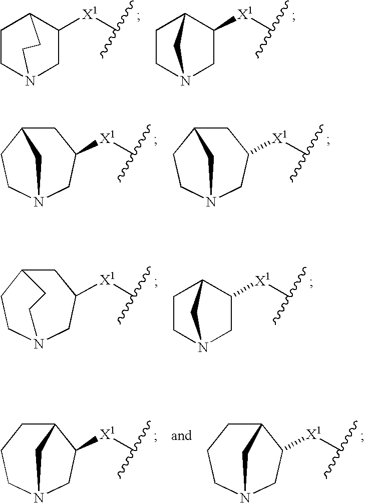
wherein X¹ is as defined for compounds of formula (I), and enantiomers thereof. Preferably, X¹ is O or NR⁹, wherein R⁹ is hydrogen or alkyl. Wherein one of A or B is a group of formula (c) wherein l is 0 and m is 2, and Y¹ is —O—, —S—, or —N(R¹¹)— in a compound of formula (I), then preferably Y² is other than a bond.

Specific examples of rings suitable for a group of formula (d) include, but are not limited to, 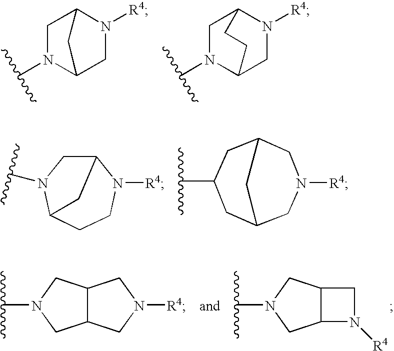
and enantiomers thereof, wherein R⁴ is as defined for compounds of formula (I). Preferably, R⁴ is hydrogen. Wherein one of A or B is selected from a group of formula (d), it can be particularly beneficial if the other of A or B is a group selected from amino, dialkylamino, and acylamino. Particularly, it is preferred that the group of formula (d) is 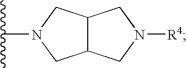
wherein R⁴ is hydrogen or alkyl. More particularly, it can be beneficial that the one of A or B is a group of formula (d) and the other is amino.

Specific examples of rings suitable for a group of formula (e) include, but are not limited to, 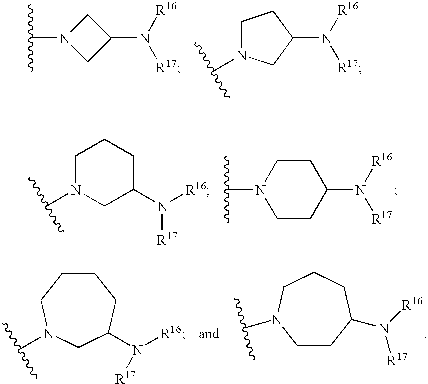
wherein R¹⁶ and R¹⁷ are as designed for components of formulas (I); and enantiomers thereof.

Specific examples of rings suitable for a group of formula (f) include, but are not limited to, 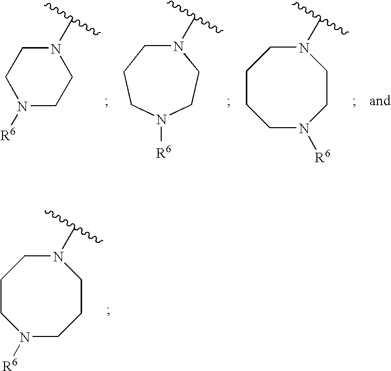
and enantiometers thereof, wherein R⁶ is as defined for compounds of formulas (I)

Specific examples of rings suitable for a group of formula (g) include, but are not limited to, 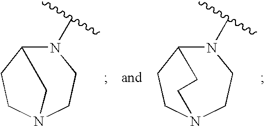
and enantiomers thereof.

Specific compounds of formula (I) contemplated as part of the invention include, but are not limited to:
2,7-bis-[(3R)-1-azabicyclo[2.2.2]octan-3-yloxy]-fluoren-9-one;
2,7-bis[(3S)-1-azabicyclo[2.2.2]octan-3-yloxy]-fluoren-9-one;
2-[(3R)-1-azabicyclo[2.2.2]octan-3-yloxy]-fluoren-9-one;
2-[(3S)-1-azabicyclo[2.2.2]octan-3-yloxy]-fluoren-9-one;
2,7-bis(4-methyl-[1,4]diazepan-1-yl)-fluoren-9-one;
2,7-bis[3,7-diazabicyclo[3.3.0]octan-3-yl]-fluoren-9-one;
2,7-bis[7-methyl-3,7-diazabicyclo[3.3.0]octan-3-yl]fluoren-9-one;
2-[3,7-diazabicyclo[3.3.0]octan-3-yl]-fluoren-9-one;
2-[7-methyl-3,7-diazabicyclo[3.3.0]octan-3-yl]-fluoren-9-one;
2,7-bis(3-diethylamino-propyn-1-yl)-fluoren-9-one;
3,7-bis(2-diethylaminoethoxy)dibenzothiophene;
3,7-bis(2-diethylaminoethoxy)dibenzothiophene-5-oxide;
3,7-bis[(3S)-1-azabicyclo[2.2.2]octan-3-yloxy]-dibenzothiophene;
2-[(1S,5S)-3,6-diazabicyclo[3.2.0]heptan-3-yl]-dibenzothiophene-5,5-dioxide;
2-amino-7-[3,7-diazabicyclo[3.3.0]octan-3-yl]-fluoren-9-one;
2-[(3R)-1-azabicyclo[2.2.2]octan-3-yloxy]-xanthen-9-one;
2-(1-azabicyclo[2.2.2]octan-3-yloxy)-9H-carbazole;
2-(3,7-diazabicyclo[3.3.0]octan-3-yl)-7-methylamino-fluoren-9-one;
2-(3,7-diazabicyclo[3.3.0]octan-3-yl)-7-dimethylamino-fluoren-9-one;
2-amino-7-(7-methyl-3,7-diazabicyclo[3.3.0]octan-3-yl)-fluoren-9-one;
2-methylamino-7-(7-methyl-3,7-diazabicyclo[3.3.0]octan-3-yl)-fluoren-9-one;
2-dimethylamino-7-(7-methyl-3,7-diazabicyclo[3.3.0]octan-3-yl)-fluoren-9-one;
2-(3,7-diazabicyclo[3.3.0]octan-3-yl)-xanthen-9-one;
2-(7-methyl-3,7-diazabicyclo[3.3.0]octan-3-yl)-xanthen-9-one;
2-amino-7-(3,7-diazabicyclo[3.3.0]octan-3-yl)-xanthen-9-one;
2-amino-7-(7-methyl-3,7-diazabicyclo[3.3.0]octan-3-yl)-xanthen-9-one;
2-(3,7-diazabicyclo[3.3.0]octan-3-yl)-7-methylamino-xanthen-9-one;
2-methylamino-7-(7-methyl-3,7-diazabicyclo[3.3.0]octan-3-yl)-xanthen-9-one;
2-(3,7-diazabicyclo[3.3.0]octan-3-yl)-7-dimethylamino-xanthen-9-one;
2-dimethylamino-7-(7-methyl-3,7-diazabicyclo[3.3.0]octan-3-yl)-xanthen-9-one;
2-amino-7-[(3R)-1-azabicyclo[2.2.2]octan-3-yloxy]-fluoren-9-one;
2-amino-7-[(3S)-1-azabicyclo[2.2.2]octan-3-yloxy]-fluoren-9-one;
2-[(3R)-1-azabicyclo[2.2.2]octan-3-yloxy]-7-methylamino-fluoren-9-one;
2-[(3S)-1-azabicyclo[2.2.2]octan-3-yloxy]-7-methylamino-fluoren-9-one;
2-[(3R)-1-azabicyclo[2.2.2]octan-3-yloxy]-7-dimethylamino-fluoren-9-one;
2-[(3S)-1-azabicyclo[2.2.2]octan-3-yloxy]-7-dimethylamino-fluoren-9-one;
3,7-bis[(3R)-1-azabicyclo[2.2.2]octan-3-yloxy]-dibenzothiophene;
3,7-bis[(3S)-1-azabicyclo[2.2.2]octan-3-yloxy]-dibenzothiophene-5,5-dioxide;
3,7-bis[(3R)-1-azabicyclo[2.2.2]octan-3-yloxy]-dibenzothiophene-5,5-dioxide;
3,7-bis[3,7-diazabicyclo[3.3.0]octan-3-yl]-dibenzothiophene-5,5-dioxide;
3,7-bis[7-methyl-3,7-diazabicyclo[3.3.0]octan-3-yl]-dibenzothiophene-5,5-dioxide;
3-[3,7-diazabicyclo[3.3.0]octan-3-yl]-dibenzothiophene-5,5-dioxide;
3-[7-methyl-3,7-diazabicyclo[3.3.0]octan-3-yl]-dibenzothiophene-5,5-dioxide;
3-amino-7-[3,7-diazabicyclo[3.3.0]octan-3-yl]-dibenzothiophene-5,5-dioxide; and
2-[(3S)-1-azabicyclo[2.2.2]octan-3-yloxy]-xanthen-9-one;
   or pharmaceutically acceptable salts, esters, amides, and prodrugs thereof.

Compounds of the invention may exist as stereoisomers wherein, asymmetric or chiral centers are present. These stereoisomers are “R” or “S” depending on the configuration of substituents around the chiral element. The terms “R” and “S” used herein are configurations as defined in IUPAC 1974 Recommendations for Section E, Fundamental Stereochemistry, Pure Appl. Chem., 1976, 45: 13-30. The invention contemplates various stereoisomers and mixtures thereof and are specifically included within the scope of this invention. Stereoisomers include enantiomers and diastereomers, and mixtures of enantiomers or diastereomers. Individual stereoisomers of compounds of the invention may be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

Methods of the Invention

Compounds and compositions of the invention are useful for modulating the effects of nAChRs, and more particularly α7 nAChRs. In particular, the compounds and compositions of the invention can be used for treating or preventing disorders modulated by α7 nAChRs. Typically, such disorders can be ameliorated by selectively modulating the α7 nAChRs in a mammal, preferably by administering a compound or composition of the invention, either alone or in combination with another active agent, for example, as part of a therapeutic regimen.

In addition, the invention relates to a method for treating or preventing a condition or disorder modulated by an α7 nicotinic acetylcholine receptor comprising the step of administering a compound of the formula (II): 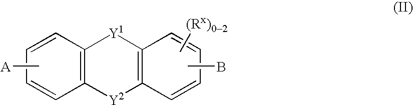
or a pharmaceutically acceptable salt, ester, amide, or prodrug thereof, wherein:
A and B are each independently selected from the group consisting of hydrogen; halogen; alkoxy; amino; alkylamino; acylamino; dialkylamino; cyano; nitro; and —SO₃H; and
a group of formula (a): 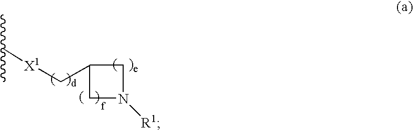
a group of formula (b): 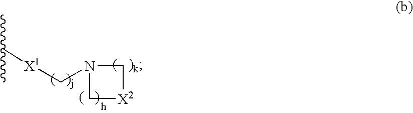
a group of formula (c): 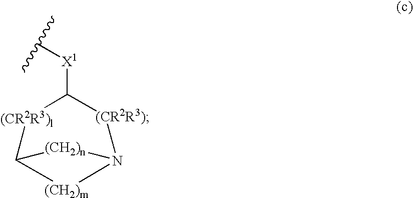
a group of formula (d): 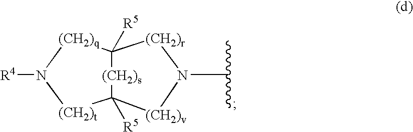
a group of formula (e): 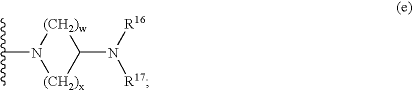
a group of formula (f): 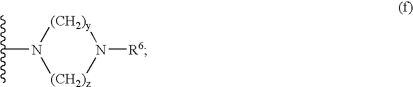
a group of formula (g): 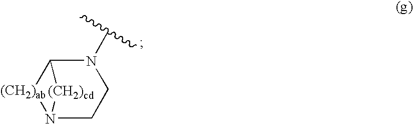
(h) —C≡CCH₂NR⁷R⁸; (i) —O—(C(R²⁰)₂₋₃N(R²¹)(R²²); and
(j) —O—(C(R²³)₂₋₃N⁺(R²⁴)(R²⁵)(R²⁶);
   X¹ at each occurrence is selected from the group consisting of O, S, and —N(R⁹)—;
   X² at each occurrence is selected from the group consisting of O, S, —CH₂—, and —N(R¹⁰)—;
   Y¹ is independently selected from the group consisting of —C(O)—, —CH₂—, —CH(OH)—, —C(S)—, —N(R¹¹)—, —O—, —S—, —S(O)—, —S(O)₂—, —C(O)NH—, and —S(O)₂NH—;
   Y² is a bond or Y² is independently selected from —O—, —S—, and —N(R¹²)—;
   R¹ is independently selected from hydrogen and alkyl;
   R² and R³ at each occurrence are each independently selected from the group consisting of hydrogen and alkyl;
   R⁴ and R⁶ at each occurrence are each independently selected from the group consisting of hydrogen and alkyl;
   R⁵ at each occurrence are each independently selected from the group consisting of hydrogen, alkyl, and alkoxycarbonyl;
   R⁷ and R⁸ are each independently selected from hydrogen and alkyl or R⁷ and R⁸ taken together with the nitrogen atom to which each is attached form a 4- to 8-membered cyclic amine;
   R⁹, R¹⁰, R¹¹, and R¹² at each occurrence are each independently selected from hydrogen and alkyl;
   R¹⁶ and R¹⁷ are each independently selected from hydrogen and alkyl, or R¹⁶ and R¹⁷ taken together with the nitrogen atom to which each is attached form a 4 to 8-membered cyclic amine;
   R²⁰ and R²³ are each independently selected from the group consisting of hydrogen and alkyl;
   R²¹ and R²² are each independently selected from the group consisting of hydrogen and alkyl;
   R²⁴, R²⁵, and R²⁶ are alkyl, or one pair of substituents selected from R²⁴, R²⁵ and R²⁶ is taken together with the nitrogen atom to which each is attached form a 4 to 8-membered cyclic amine and the remaining substituent is selected from hydrogen and alkyl;
   R^{x} is independently selected at each occurrence from the group consisting of hydrogen, halogen, alkoxy, amino, alkylamino, dialkylamino, acylamino, dialkylaminoalkyl, and cyano;
   d is independently selected from 0 or 1;
   e and f are each independently selected from 0, 1, 2 or 3, provided that the sum total of e and f is 2, 3, or 4, provided that when d is 0, e and f are selected from 1, 2 or 3;
   j is independently selected from 2 or 3;
   h and k are each independently selected from 0, 1, or 2, provided that the sum total of h and k is 2, 3, or 4, provided that when X² is O, S, or N(R¹⁰), h and k are both 2;
   l is 0 or 1, m is 2 or 3, and n is 0, 1, or 2, provided that the sum total of l, m, and n is 4, 5, or 6;
   q, r, s, t, and v are each independently selected from 0, 1, or 2, provided that the sum of q and r; t and v; q, s, and t; and r, s, and v; are each at least 1;
   w and x are each independently selected from 1, 2, or 3, provided that the sum total of w and x is 3, 4, 5, or 6;
   y and z are each independently selected from 2, 3, or 4, provided that the sum total of y and z is 4, 5, or 6; and
   ab is 2 or 3, and cd is 1 or 2.

Preferred compounds for the method of the invention are those wherein the group A, the group B, or both groups A and B are selected from the group consisting of substituents (a)-(j). More preferably, the compound for the method is one wherein the group A, the group B, or both groups A and B is a substituent (d). When one of A or B is selected from a group of formula (d) it can be beneficial that the other is selected from amino, dialkylamino, and acylamino. It is particularly preferred when the group of formula (d) is 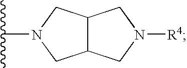
wherein R⁴ is hydrogen or alkyl, and more particularly, when the other substituent of A or B is amino.

Compounds for the method of the invention, including but not limited to those specified in the examples or otherwise specifically named, can modulate, and often possess an affinity for, nAChRs, and more particularly α7 nAChRs. As α7nAChRs ligands, the compounds of the invention can be useful for the treatment or prevention of a number of α7 nAChR-mediated diseases or conditions.

Specific examples of compounds that can be useful for the treatment or prevention of α7 nAChR-mediated diseases or conditions include, but are not limited to, compounds described in the Examples, such as
2,7-bis[(2R)-1-methylpyrrolidin-2-ylmethoxy]-fluoren-9-one;
2,7-bis[(2R)-azetidin-2-ylmethoxy]-fluoren-9-one;
2,7-bis[(2R)-1-methylazetidin-2-ylmethoxy]-fluoren-9-one;
2,7-bis[(3S)-pyrrolidin-3-yloxy]-fluoren-9-one;
2,7-bis[(3S)-1-methylpyrrolidin-3-yloxy]-fluoren-9-one;
2,7-bis-[(3R)-1-azabicyclo[2.2.2]octan-3-yloxy]-fluoren-9-one;
2,7-bis[(3S)-1-azabicyclo[2.2.2]octan-3-yloxy]-fluoren-9-one;
2-[(3R)-1-azabicyclo[2.2.2]octan-3-yloxy]-fluoren-9-one;
2-[(3S)-1-azabicyclo[2.2.2]octan-3-yloxy]-fluoren-9-one;
2,7-bis(4-methyl-[1,4]diazepan-1-yl)-fluoren-9-one;
2,7-bis[3,7-diazabicyclo[3.3.0]octan-3-yl]-fluoren-9-one;
2,7-bis[7-methyl-3,7-diazabicyclo[3.3.0]octan-3-yl]fluoren-9-one;
2-[3,7-diazabicyclo[3.3.0]octan-3-yl]-fluoren-9-one;
2-[7-methyl-3,7-diazabicyclo[3.3.0]octan-3-yl]-fluoren-9-one;
2,7-bis(3-diethylamino-propyn-1-yl)-fluoren-9-one;
3,7-bis(2-diethylaminoethoxy)dibenzothiophene;
3,7-bis(2-diethylaminoethoxy)dibenzothiophene-5-oxide;
3,7-bis[(3S)-1-azabicyclo[2.2.2]octan-3-yloxy]-dibenzothiophene;
2-[(1S,5S)-3,6-diazabicyclo[3.2.0]heptan-3-yl]-dibenzothiophene-5,5-dioxide;
2-amino-7-[3,7-diazabicyclo[3.3.0]octan-3-yl]-fluoren-9-one;
2-[(3R)-1-azabicyclo[2.2.2]octan-3-yloxy]-xanthen-9-one; and
2-(1-azabicyclo[2.2.2]octan-3-yloxy)-9H-carbazole;
   and pharmaceutically acceptable salts, esters, amides, and prodrugs thereof. Additionally, compounds that can be prepared by methods described in the Schemes and methods readily available to one with skill in the art include, but are not limited to, for example,
2,7-bis-(2-aminoethoxy)-fluorene;
2,7-bis-(3-aminopropoxy)-fluorene;
2,7-bis-(2-methylaminoethoxy)-fluorene;
2,7-bis-(2-ethylaminoethoxy)-fluorene;
2,7-bis-(2-n-propylaminoethoxy)-fluorene;
2,7-bis-(3-methylaminopropoxy)-fluorene;
2,7-bis-(3-ethylaminopropoxy)-fluorene;
2,7-bis-(3-n-propylaminopropoxy)-fluorene;
2,7-bis-(2-dimethylaminoethoxy)-fluorene;
2,7-bis-(2-diethylaminoethoxy)-fluorene;
2,7-bis-(2-di-n-propylaminoethoxy)-fluorene;
2,7-bis-(3-dimethylaminopropoxy)-fluorene;
2,7-bis-(3-diethylaminopropoxy)-fluorene;
2,7-bis-(3-di-n-propylaminopropoxy)-fluorene;
2,7-bis-(2-azetidin-1-yl-ethoxy)-fluorene;
2,7-bis-(2-pyrrolidin-1-yl-ethoxy)-fluorene;
2,7-bis-(2-piperidin-1-yl-ethoxy)-fluorene;
2,7-bis-(3-azetidin-1-yl-propoxy)-fluorene;
2,7-bis-(3-pyrrolidin-1-yl-propoxy)-fluorene;
2,7-bis-(3-piperidin-1-yl-propoxy)-fluorene;
2,7-bis-(2-trimethylammoniumethoxy)-fluorene;
2,7-bis-(3-trimethylammoniumpropoxy)-fluorene;
2,6-bis-(2-aminoethoxy)fluorene;
2,6-bis-(3-aminopropoxy)fluorene;
2,6-bis-(2-methylaminoethoxy)-fluorene;
2,6-bis-(2-ethylaminoethoxy)-fluorene;
2,6-bis-(2-n-propylaminoethoxy)-fluorene;
2,6-bis-(3-methylaminopropoxy)-fluorene;
2,6-bis-(3-ethylaminopropoxy)-fluorene;
2,6-bis-(3-n-propylaminopropoxy)-fluorene;
2,6-bis-(2-dimethylaminoethoxy)-fluorene;
2,6-bis-(2-diethylaminoethoxy)-fluorene;
2,6-bis-(2-di-n-propylaminoethoxy)-fluorene;
2,6-bis-(3-dimethylaminopropoxy)-fluorene;
2,6-bis-(3-diethylaminopropoxy)-fluorene;
2,6-bis-(3-di-n-propylaminopropoxy)-fluorene;
2,6-bis-(2-azetidin-1-yl-ethoxy)-fluorene;
2,6-bis-(2-pyrrolidin-1-yl-ethoxy)-fluorene;
2,6-bis-(2-piperidin-1-yl-ethoxy)-fluorene;
2,6-bis-(3-azetidin-1-yl-propoxy)-fluorene;
2,6-bis-(3-pyrrolidin-1-yl-propoxy)-fluorene;
2,6-bis-(3-piperidin-1-yl-propoxy)-fluorene;
2,6-bis-(2-trimethylammoniumethoxy)-fluorene;
2,6-bis-(3-trimethylammoniumpropoxy)-fluorene;
3,6-bis-(2-aminoethoxy)-fluorene;
3,6-bis-(3-aminopropoxy)-fluorene;
3,6-bis-(2-methylaminoethoxy)-fluorene;
3,6-bis-(2-ethylaminoethoxy)-fluorene;
3,6-bis-(2-n-propylaminoethoxy)-fluorene;
3,6-bis-(3-methylaminopropoxy)-fluorene;
3,6-bis-(3-ethylaminopropoxy)-fluorene;
3,6-bis-(3-n-propylaminopropoxy)-fluorene;
3,6-bis-(2-dimethylaminoethoxy)-fluorene;
3,6-bis-(2-diethylaminoethoxy)-fluorene;
3,6-bis-(2-di-n-propylaminoethoxy)-fluorene;
3,6-bis-(3-dimethylaminopropoxy)-fluorene;
3,6-bis-(3-diethylaminopropoxy)-fluorene;
3,6-bis-(3-di-n-propylaminopropoxy)-fluorene;
3,6-bis-(2-azetidin-1-yl-ethoxy)-fluorene;
3,6-bis-(2-pyrrolidin-1-yl-ethoxy)-fluorene;
3,6-bis-(2-piperidin-1-yl-ethoxy)-fluorene;
3,6-bis-(3-azetidin-1-yl-propoxy)-fluorene;
3,6-bis-(3-pyrrolidin-1-yl-propoxy)-fluorene;
3,6-bis-(3-piperidin-1-yl-propoxy)-fluorene;
3,6-bis-(2-trimethylammoniumethoxy)-fluorene;
3,6-bis-(3-trimethylammoniumpropoxy)-fluorene;
2,7-bis-(2-aminoethoxy)-fluoren-9-ol;
2,7-bis-(3-aminopropoxy)fluoren-9-ol;
2,7-bis-(2-methylaminoethoxy)-fluoren-9-ol;
2,7-bis-(2-ethylaminoethoxy)-fluoren-9-ol;
2,7-bis-(2-n-propylaminoethoxy)-fluoren-9-ol;
2,7-bis-(3-methylaminopropoxy)-fluoren-9-ol;
2,7-bis-(3-ethylaminopropoxy)-fluoren-9-ol;
2,7-bis-(3-n-propylaminopropoxy)-fluoren-9-ol;
2,7-bis-(2-dimethylaminoethoxy)-fluoren-9-ol;
2,7-bis-(2-diethylaminoethoxy)-fluoren-9-ol;
2,7-bis-(2-di-n-propylaminoethoxy)-fluoren-9-ol;
2,7-bis-(3-dimethylaminopropoxy)-fluoren-9-ol;
2,7-bis-(3-diethylaminopropoxy)-fluoren-9-ol;
2,7-bis-(3-di-n-propylaminopropoxy)-fluoren-9-ol;
2,7-bis-(2-azetidin-1-yl-ethoxy)-fluoren-9-ol;
2,7-bis-(2-pyrrolidin-1-yl-ethoxy)-fluoren-9-ol;
2,7-bis-(2-piperidin-1-yl-ethoxy)-fluoren-9-ol;
2,7-bis-(3-azetidin-1-yl-propoxy)-fluoren-9-ol;
2,7-bis-(3-pyrrolidin-1-yl-propoxy)-fluoren-9-ol;
2,7-bis-(3-piperidin-1-yl-propoxy)-fluoren-9-ol;
2,7-bis-(2-trimethylammoniumethoxy)-fluoren-9-ol;
2,7-bis-(3-trimethylammoniumpropoxy)-fluoren-9-ol;
2,6-bis-(2-aminoethoxy)fluoren-9-ol;
2,6-bis-(3-aminopropoxy)fluoren-9-ol;
2,6-bis-(2-methylaminoethoxy)-fluoren-9-ol;
2,6-bis-(2-ethylaminoethoxy)-fluoren-9-ol;
2,6-bis-(2-n-propylaminoethoxy)-fluoren-9-ol;
2,6-bis-(3-methylaminopropoxy)-fluoren-9-ol;
2,6-bis-(3-ethylaminopropoxy)-fluoren-9-ol;
2,6-bis-(3-n-propylaminopropoxy)-fluoren-9-ol;
2,6-bis-(2-dimethylaminoethoxy)-fluoren-9-ol;
2,6-bis-(2-diethylaminoethoxy)-fluoren-9-ol;
2,6-bis-(2-di-n-propylaminoethoxy)-fluoren-9-ol;
2,6-bis-(3-dimethylaminopropoxy)-fluoren-9-ol;
2,6-bis-(3-diethylaminopropoxy)-fluoren-9-ol;
2,6-bis-(3-di-n-propylaminopropoxy)-fluoren-9-ol;
2,6-bis-(2-azetidin-1-yl-ethoxy)-fluoren-9-ol;
2,6-bis-(2-pyrrolidin-1-yl-ethoxy)-fluoren-9-ol;
2,6-bis-(2-piperidin-1-yl-ethoxy)-fluoren-9-ol;
2,6-bis-(3-azetidin-1-yl-propoxy)-fluoren-9-ol;
2,6-bis-(3-pyrrolidin-1-yl-propoxy)-fluoren-9-ol;
2,6-bis-(3-piperidin-1-yl-propoxy)-fluoren-9-ol;
2,6-bis-(2-trimethylammoniumethoxy)-fluoren-9-ol;
2,6-bis-(3-trimethylammoniumpropoxy)-fluoren-9-ol;
3,6-bis-(2-aminoethoxy)fluoren-9-ol;
3,6-bis-(3-aminopropoxy)fluoren-9-ol;
3,6-bis-(2-methylaminoethoxy)-fluoren-9-ol;
3,6-bis-(2-ethylaminoethoxy)-fluoren-9-ol;
3,6-bis-(2-n-propylaminoethoxy)-fluoren-9-ol;
3,6-bis-(3-methylaminopropoxy)-fluoren-9-ol;
3,6-bis-(3-ethylaminopropoxy)-fluoren-9 ol;
3,6-bis-(3-n-propylaminopropoxy)-fluoren-9-ol;
3,6-bis-(2-dimethylaminoethoxy)-fluoren-9-ol;
3,6-bis-(2-diethylaminoethoxy)-fluoren-9-ol;
3,6-bis-(2-di-n-propylaminoethoxy)-fluoren-9-ol;
3,6-bis-(3-dimethylaminopropoxy)-fluoren-9-ol;
3,6-bis-(3-diethylaminopropoxy)-fluoren-9-ol;
3,6-bis-(3-di-n-propylaminopropoxy)-fluoren-9-ol;
3,6-bis-(2-azetidin-1-yl-ethoxy)-fluoren-9-ol;
3,6-bis-(2-pyrrolidin-1-yl-ethoxy)-fluoren-9-ol;
3,6-bis-(2-piperidin-1-yl-ethoxy)-fluoren-9-ol;
3,6-bis-(3-azetidin-1-yl-propoxy)-fluoren-9-ol;
3,6-bis-(3-pyrrolidin-1-yl-propoxy)-fluoren-9-ol;
3,6-bis-(3-piperidin-1-yl-propoxy)-fluoren-9-ol;
3,6-bis-(2-trimethylammoniumethoxy)-fluoren-9-ol;
3,6-bis-(3-trimethylammoniumpropoxy)-fluoren-9-ol;
2,7-bis-(2-aminoethoxy)-fluoren-9-one;
2,7-bis-(3-aminopropoxy)fluoren-9-one;
2,7-bis-(2-methylaminoethoxy)-fluoren-9-one;
2,7-bis-(2-ethylaminoethoxy)-fluoren-9-one;
2,7-bis-(2-n-propylaminoethoxy)-fluoren-9-one;
2,7-bis-(3-methylaminopropoxy)-fluoren-9-one;
2,7-bis-(3-ethylaminopropoxy)-fluoren-9-one;
2,7-bis-(3-n-propylaminopropoxy)-fluoren-9-one;
2,7-bis-(2-dimethylaminoethoxy)-fluoren-9-one;
2,7-bis-(2-diethylaminoethoxy)-fluoren-9-one;
2,7-bis-(2-di-n-propylaminoethoxy)-fluoren-9-one;
2,7-bis-(3-dimethylaminopropoxy)-fluoren 9-one;
2,7-bis-(3-diethylaminopropoxy)-fluoren-9-one;
2,7-bis-(3-di-n-propylaminopropoxy)-fluoren-9-one;
2,7-bis-(2-azetidin-1-yl-ethoxy)-fluoren-9-one;
2,7-bis-(2-pyrrolidin-1-yl-ethoxy)-fluoren-9-one;
2,7-bis-(2-piperidin-1-yl-ethoxy)-fluoren-9-one;
2,7-bis-(3-azetidin-1-yl-propoxy)-fluoren-9-one;
2,7-bis-(3-pyrrolidin-1-yl-propoxy)-fluoren-9-one;
2,7-bis-(3-piperidin-1-yl-propoxy)-fluoren-9-one;
2,7-bis-(2-trimethylammoniumethoxy)-fluoren-9-one;
2,7-bis-(3-trimethylammoniumpropoxy)-fluoren-9-one;
2,6-bis-(2-aminoethoxy)-fluoren-9-one;
2,6-bis-(3-aminopropoxy)-fluoren-9-one;
2,6-bis-(2-methylaminoethoxy)-fluoren-9-one;
2,6-bis-(2-ethylaminoethoxy)-fluoren-9-one;
2,6-bis-(2-n-propylaminoethoxy)-fluoren-9-one;
2,6-bis-(3-methylaminopropoxy)-fluoren-9-one;
2,6-bis-(3-ethylaminopropoxy)-fluoren-9-one;
2,6-bis-(3-n-propylaminopropoxy)-fluoren-9-one;
2,6-bis-(2-dimethylaminoethoxy)-fluoren-9-one;
2,6-bis-(2-diethylaminoethoxy)-fluoren-9-one;
2,6-bis-(2-di-n-propylaminoethoxy)-fluoren-9-one;
2,6-bis-(3-dimethylaminopropoxy)-fluoren-9-one;
2,6-bis-(3-diethylaminopropoxy)-fluoren-9-one;
2,6-bis-(3-di-n-propylaminopropoxy)-fluoren-9-one;
2,6-bis-(2-azetidin-1-yl-ethoxy)-fluoren-9-one;
2,6-bis-(2-pyrrolidin-1-yl-ethoxy)-fluoren-9-one;
2,6-bis-(2-piperidin-1-yl-ethoxy)-fluoren-9-one;
2,6-bis-(3-azetidin-1-yl-propoxy)-fluoren-9-one;
2,6-bis-(3-pyrrolidin-1-yl-propoxy)-fluoren-9-one;
2,6-bis-(3-piperidin-1-yl-propoxy)-fluoren-9-one;
2,6-bis-(2-trimethylammoniumethoxy)-fluoren-9-one;
2,6-bis-(3-trimethylammoniumpropoxy)-fluoren-9-one;
3,6-bis-(2-aminoethoxy)fluoren-9-one;
3,6-bis-(3-aminopropoxy)fluoren-9-one;
3,6-bis-(2-methylaminoethoxy)-fluoren-9-one;
3,6-bis-(2-ethylaminoethoxy)-fluoren-9-one;
3,6-bis-(2-n-propylaminoethoxy)-fluoren-9-one;
3,6-bis-(3-methylaminopropoxy)-fluoren-9-one;
3,6-bis-(3-ethylaminopropoxy)-fluoren-9-one;
3,6-bis-(3-n-propylaminopropoxy)-fluoren-9-one;
3,6-bis-(2-dimethylaminoethoxy)-fluoren-9-one;
3,6-bis-(2-diethylaminoethoxy)-fluoren-9-one;
3,6-bis-(2-di-n-propylaminoethoxy)-fluoren-9-one;
3,6-bis-(3-dimethylaminopropoxy)-fluoren-9-one;
3,6-bis-(3-diethylaminopropoxy)-fluoren-9-one;
3,6-bis-(3-di-n-propylaminopropoxy)-fluoren-9-one;
3,6-bis-(2-azetidin-1-yl-ethoxy)-fluoren-9-one;
3,6-bis-(2-pyrrolidin-1-yl-ethoxy)-fluoren-9-one;
3,6-bis-(2-piperidin-1-yl-ethoxy)-fluoren-9-one;
3,6-bis-(3-azetidin-1-yl-propoxy)-fluoren-9-one;
3,6-bis-(3-pyrrolidin-1-yl-propoxy)-fluoren-9-one;
3,6-bis-(3-piperidin-1-yl-propoxy)-fluoren-9-one;
3,6-bis-(2-trimethylammoniumethoxy)-fluoren-9-one;
3,6-bis-(3-trimethylammoniumpropoxy)-fluoren-9-one;
3,7-bis-(2-aminoethoxy)-dibenzofuran;
3,7-bis-(3-aminopropoxy)-dibenzofuran;
3,7-bis-(2-methylaminoethoxy)-dibenzofuran;
3,7-bis-(2-ethylaminoethoxy)-dibenzofuran;
3,7-bis-(2-n-propylaminoethoxy)-dibenzofuran;
3,7-bis-(3-methylaminopropoxy)-dibenzofuran;
3,7-bis-(3-ethylaminopropoxy)-dibenzofuran;
3,7-bis-(3-n-propylaminopropoxy)-dibenzofuran;
3,7-bis-(2-dimethylaminoethoxy)-dibenzofuran;
3,7-bis-(2-diethylaminoethoxy)-dibenzofuran;
3,7-bis-(2-di-n-propylaminoethoxy)-dibenzofuran;
3,7-bis-(3-dimethylaminopropoxy)-dibenzofuran;
3,7-bis-(3-diethylaminopropoxy)-dibenzofuran;
3,7-bis-(3-di-n-propylaminopropoxy)-dibenzofuran;
3,7-bis-(2-azetidin-1-yl-ethoxy)-dibenzofuran;
3,7-bis-(2-pyrrolidin-1-yl-ethoxy)-dibenzofuran;
3,7-bis-(2-piperidin-1-yl-ethoxy)-dibenzofuran;
3,7-bis-(3-azetidin-1-yl-propoxy)-dibenzofuran;
3,7-bis-(3-pyrrolidin-1-yl-propoxy)-dibenzofuran;
3,7-bis-(3-piperidin-1-yl-propoxy)-dibenzofuran;
3,7-bis-(2-trimethylammoniumethoxy)-dibenzofuran;
3,7-bis-(3-trimethylammoniumpropoxy)-dibenzofuran;
2,7-bis-(2-aminoethoxy)-dibenzofuran;
2,7-bis-(3-aminopropoxy)-dibenzofuran;
2,7-bis-(2-methylaminoethoxy)-dibenzofuran;
2,7-bis-(2-ethylaminoethoxy)-dibenzofuran;
2,7-bis-(2-n-propylaminoethoxy)-dibenzofuran;
2,7-bis-(3-methylaminopropoxy)-dibenzofuran;
2,7-bis-(3-ethylaminopropoxy)-dibenzofuran;
2,7-bis-(3-n-propylaminopropoxy)-dibenzofuran;
2,7-bis-(2-dimethylaminoethoxy)-dibenzofuran;
2,7-bis-(2-diethylaminoethoxy)-dibenzofuran;
2,7-bis-(2-di-n-propylaminoethoxy)-dibenzofuran;
2,7-bis-(3-dimethylaminopropoxy)-dibenzofuran;
2,7-bis-(3-diethylaminopropoxy)-dibenzofuran;
2,7-bis-(3-di-n-propylaminopropoxy)-dibenzofuran;
2,7-bis-(2-azetidin-1-yl-ethoxy)-dibenzofuran;
2,7-bis-(2-pyrrolidin-1-yl-ethoxy)-dibenzofuran;
2,7-bis-(2-piperidin-1-yl-ethoxy)-dibenzofuran;
2,7-bis-(3-azetidin-1-yl-propoxy)-dibenzofuran;
2,7-bis-(3-pyrrolidin-1-yl-propoxy)-dibenzofuran;
2,7-bis-(3-piperidin-1-yl-propoxy)-dibenzofuran;
2,7-bis-(2-trimethylammoniumethoxy)-dibenzofuran;
2,7-bis-(3-trimethylammoniumpropoxy)-dibenzofuran;
2,8-bis-(2-aminoethoxy)-dibenzofuran;
2,8-bis-(3-aminopropoxy)-dibenzofuran;
2,8-bis-(2-methylaminoethoxy)-dibenzofuran;
2,8-bis-(2-ethylaminoethoxy)-dibenzofuran;
2,8-bis-(2-n-propylaminoethoxy)-dibenzofuran;
2,8-bis-(3-methylaminopropoxy)-dibenzofuran;
2,8-bis-(3-ethylaminopropoxy)-dibenzofuran;
2,8-bis-(3-n-propylaminopropoxy)-dibenzofuran;
2,8-bis-(2-dimethylaminoethoxy)-dibenzofuran;
2,8-bis-(2-diethylaminoethoxy)-dibenzofuran;
2,8-bis-(2-di-n-propylaminoethoxy)-dibenzofuran;
2,8-bis-(3-dimethylaminopropoxy)-dibenzofuran;
2,8-bis-(3-diethylaminopropoxy)-dibenzofuran;
2,8-bis-(3-di-n-propylaminopropoxy)-dibenzofuran;
2,8-bis-(2-azetidin-1-yl-ethoxy)-dibenzofuran;
2,8-bis-(2-pyrrolidin-1-yl-ethoxy)-dibenzofuran;
2,8-bis-(2-piperidin-1-yl-ethoxy)-dibenzofuran;
2,8-bis-(3-azetidin-1-yl-propoxy)-dibenzofuran;
2,8-bis-(3-pyrrolidin-1-yl-propoxy)-dibenzofuran;
2,8-bis-(3-piperidin-1-yl-propoxy)-dibenzofuran;
2,8-bis-(2-trimethylammoniumethoxy)-dibenzofuran;
2,8-bis-(3-trimethylammoniumpropoxy)-dibenzofuran;
3,7-bis-(2-aminoethoxy)-dibenzothiophene;
3,7-bis-(3-aminopropoxy)-dibenzothiophene;
3,7-bis-(2-methylaminoethoxy)-dibenzothiophene;
3,7-bis-(2-ethylaminoethoxy)-dibenzothiophene;
3,7-bis-(2-n-propylaminoethoxy)-dibenzothiophene;
3,7-bis-(3-methylaminopropoxy)-dibenzothiophene;
3,7-bis-(3-ethylaminopropoxy)-dibenzothiophene;
3,7-bis-(3-n-propylaminopropoxy)-dibenzothiophene;
3,7-bis-(2-dimethylaminoethoxy)-dibenzothiophene;
3,7-bis-(2-diethylaminoethoxy)-dibenzothiophene;
3,7-bis-(2-di-n-propylaminoethoxy)-dibenzothiophene;
3,7-bis-(3-dimethylaminopropoxy)-dibenzothiophene;
3,7-bis-(3-diethylaminopropoxy)-dibenzothiophene;
3,7-bis-(3-di-n-propylaminopropoxy)-dibenzothiophene;
3,7-bis-(2-azetidin-1-yl-ethoxy)-dibenzothiophene;
3,7-bis-(2-pyrrolidin-1-yl-ethoxy)-dibenzothiophene;
3,7-bis-(2-piperidin-1-yl-ethoxy)-dibenzothiophene;
3,7-bis-(3-azetidin-1-yl-propoxy)-dibenzothiophene;
3,7-bis-(3-pyrrolidin-1-yl-propoxy)-dibenzothiophene;
3,7-bis-(3-piperidin-1-yl-propoxy)-dibenzothiophene;
3,7-bis-(2-trimethylammoniumethoxy)-dibenzothiophene;
3,7-bis-(3-trimethylammoniumpropoxy)-dibenzothiophene;
2,7-bis-(2-aminoethoxy)-dibenzothiophene;
2,7-bis-(3-aminopropoxy)-dibenzothiophene;
2,7-bis-(2-methylaminoethoxy)-dibenzothiophene;
2,7-bis-(2-ethylaminoethoxy)-dibenzothiophene;
2,7-bis-(2-n-propylaminoethoxy)-dibenzothiophene;
2,7-bis-(3-methylaminopropoxy)-dibenzothiophene;
2,7-bis-(3-ethylaminopropoxy)-dibenzothiophene;
2,7-bis-(3-n-propylaminopropoxy)-dibenzothiophene;
2,7-bis-(2-dimethylaminoethoxy)-dibenzothiophene;
2,7-bis-(2-diethylaminoethoxy)-dibenzothiophene;
2,7-bis-(2-di-n-propylaminoethoxy)-dibenzothiophene;
2,7-bis-(3-dimethylaminopropoxy)-dibenzothiophene;
2,7-bis-(3-diethylaminopropoxy)-dibenzothiophene;
2,7-bis-(3-di-n-propylaminopropoxy)-dibenzothiophene;
2,7-bis-(2-azetidin-1-yl-ethoxy)-dibenzothiophene;
2,7-bis-(2-pyrrolidin-1-yl-ethoxy)-dibenzothiophene;
2,7-bis-(2-piperidin-1-yl-ethoxy)-dibenzothiophene;
2,7-bis-(3-azetidin-1-yl-propoxy)-dibenzothiophene;
2,7-bis-(3-pyrrolidin-1-yl-propoxy)-dibenzothiophene;
2,7-bis-(3-piperidin-1-yl-propoxy)-dibenzothiophene;
2,7-bis-(2-trimethylammoniumethoxy)-dibenzothiophene;
2,7-bis-(3-trimethylammoniumpropoxy)-dibenzothiophene;
2,8-bis-(2-aminoethoxy)-dibenzothiophene;
2,8-bis-(3-aminopropoxy)-dibenzothiophene;
2,8-bis-(2-methylaminoethoxy)-dibenzothiophene;
2,8-bis-(2-ethylaminoethoxy)-dibenzothiophene;
2,8-bis-(2-n-propylaminoethoxy)-dibenzothiophene;
2,8-bis-(3-methylaminopropoxy)-dibenzothiophene;
2,8-bis-(3-ethylaminopropoxy)-dibenzothiophene;
2,8-bis-(3-n-propylaminopropoxy)-dibenzothiophene;
2,8-bis-(2-dimethylaminoethoxy)-dibenzothiophene;
2,8-bis-(2-diethylaminoethoxy)-dibenzothiophene;
2,8-bis-(2-di-n-propylaminoethoxy)-dibenzothiophene;
2,8-bis-(3-dimethylaminopropoxy)-dibenzothiophene;
2,8-bis-(3-diethylaminopropoxy)-dibenzothiophene;
2,8-bis-(3-di-n-propylaminopropoxy)-dibenzothiophene;
2,8-bis-(2-azetidin-1-yl-ethoxy)-dibenzothiophene;
2,8-bis-(2-pyrrolidin-1-yl-ethoxy)-dibenzothiophene;
2,8-bis-(2-piperidin-1-yl-ethoxy)-dibenzothiophene;
2,8-bis-(3-azetidin-1-yl-propoxy)-dibenzothiophene;
2,8-bis-(3-pyrrolidin-1-yl-propoxy)-dibenzothiophene;
2,8-bis-(3-piperidin-1-yl-propoxy)-dibenzothiophene;
2,8-bis-(2-trimethylammoniumethoxy)-dibenzothiophene;
2,8-bis-(3-trimethylammoniumpropoxy)-dibenzothiophene;
3,7-bis-(2-aminoethoxy)-dibenzothiophene-5-oxide;
3,7-bis-(3-aminopropoxy)-dibenzothiophene-5-oxide;
3,7-bis-(2-methylaminoethoxy)-dibenzothiophene-5-oxide;
3,7-bis-(2-ethylaminoethoxy)-dibenzothiophene-5-oxide;
3,7-bis-(2-n-propylaminoethoxy)-dibenzothiophene-5-oxide;
3,7-bis-(3-methylaminopropoxy)-dibenzothiophene-5-oxide;
3,7-bis-(3-ethylaminopropoxy)-dibenzothiophene-5-oxide;
3,7-bis-(3-n-propylaminopropoxy)-dibenzothiophene-5-oxide;
3,7-bis-(2-dimethylaminoethoxy)-dibenzothiophene-5-oxide;
3,7-bis-(2-diethylaminoethoxy)-dibenzothiophene-5-oxide;
3,7-bis-(2-di-n-propylaminoethoxy)-dibenzothiophene-5-oxide;
3,7-bis-(3-dimethylaminopropoxy)-dibenzothiophene-5-oxide;
3,7-bis-(3-diethylaminopropoxy)-dibenzothiophene-5-oxide;
3,7-bis-(3-di-n-propylaminopropoxy)-dibenzothiophene-5-oxide;
3,7-bis-(2-azetidin-1-yl-ethoxy)-dibenzothiophene-5-oxide;
3,7-bis-(2-pyrrolidin-1-yl-ethoxy)-dibenzothiophene-5-oxide;
3,7-bis-(2-piperidin-1-yl-ethoxy)-dibenzothiophene-5-oxide;
3,7-bis-(3-azetidin-1-yl-propoxy)-dibenzothiophene-5-oxide;
3,7-bis-(3-pyrrolidin-1-yl-propoxy)-dibenzothiophene-5-oxide;
3,7-bis-(3-piperidin-1-yl-propoxy)-dibenzothiophene-5-oxide;
3,7-bis-(2-trimethylammoniumethoxy)-dibenzothiophene-5-oxide;
3,7-bis-(3-trimethylammoniumpropoxy)-dibenzothiophene-5-oxide;
2,7-bis-(2-aminoethoxy)-dibenzothiophene-5-oxide;
2,7-bis-(3-aminopropoxy)-dibenzothiophene-5-oxide;
2,7-bis-(2-methylaminoethoxy)-dibenzothiophene-5-oxide;
2,7-bis-(2-ethylaminoethoxy)-dibenzothiophene-5-oxide;
2,7-bis-(2-n-propylaminoethoxy)-dibenzothiophene-5-oxide;
2,7-bis-(3-methylaminopropoxy)-dibenzothiophene-5-oxide;
2,7-bis-(3-ethylaminopropoxy)-dibenzothiophene-5-oxide;
2,7-bis-(3-n-propylaminopropoxy)-dibenzothiophene-5-oxide;
2,7-bis-(2-dimethylaminoethoxy)-dibenzothiophene-5-oxide;
2,7-bis-(2-diethylaminoethoxy)-dibenzothiophene-5-oxide;
2,7-bis-(2-di-n-propylaminoethoxy)-dibenzothiophene-5-oxide;
2,7-bis-(3-dimethylaminopropoxy)-dibenzothiophene-5-oxide;
2,7-bis-(3-diethylaminopropoxy)-dibenzothiophene-5-oxide;
2,7-bis-(3-di-n-propylaminopropoxy)-dibenzothiophene-5-oxide;
2,7-bis-(2-azetidin-1-yl-ethoxy)-dibenzothiophene-5-oxide;
2,7-bis-(2-pyrrolidin-1-yl-ethoxy)-dibenzothiophene-5-oxide;
2,7-bis-(2-piperidin-1-yl-ethoxy)-dibenzothiophene-5-oxide;
2,7-bis-(3-azetidin-1-yl-propoxy)-dibenzothiophene-5-oxide;
2,7-bis-(3-pyrrolidin-1-yl-propoxy)-dibenzothiophene-5-oxide;
2,7-bis-(3-piperidin-1-yl-propoxy)-dibenzothiophene-5-oxide;
2,7-bis-(2-trimethylammoniumethoxy)-dibenzothiophene-5-oxide;
2,7-bis-(3-trimethylammoniumpropoxy)-dibenzothiophene-5-oxide;
2,8-bis-(2-aminoethoxy)-dibenzothiophene-5-oxide;
2,8-bis-(3-aminopropoxy)-dibenzothiophene-5-oxide;
2,8-bis-(2-methylaminoethoxy)-dibenzothiophene-5-oxide;
2,8-bis-(2-ethylaminoethoxy)-dibenzothiophene-5-oxide;
2,8-bis-(2-n-propylaminoethoxy)-dibenzothiophene-5-oxide;
2,8-bis-(3-methylaminopropoxy)-dibenzothiophene-5-oxide;
2,8-bis-(3-ethylaminopropoxy)-dibenzothiophene-5-oxide;
2,8-bis-(3-n-propylaminopropoxy)-dibenzothiophene-5-oxide;
2,8-bis-(2-dimethylaminoethoxy)-dibenzothiophene-5-oxide;
2,8-bis-(2-diethylaminoethoxy)-dibenzothiophene-5-oxide;
2,8-bis-(2-di-n-propylaminoethoxy)-dibenzothiophene-5-oxide;
2,8-bis-(3-dimethylaminopropoxy)-dibenzothiophene-5-oxide;
2,8-bis-(3-diethylaminopropoxy)-dibenzothiophene-5-oxide;
2,8-bis-(3-di-n-propylaminopropoxy)-dibenzothiophene-5-oxide;
2,8-bis-(2-azetidin-1-yl-ethoxy)-dibenzothiophene-5-oxide;
2,8-bis-(2-pyrrolidin-1-yl-ethoxy)-dibenzothiophene-5-oxide;
2,8-bis-(2-piperidin-1-yl-ethoxy)-dibenzothiophene-5-oxide;
2,8-bis-(3-azetidin-1-yl-propoxy)-dibenzothiophene-5-oxide;
2,8-bis-(3-pyrrolidin-1-yl-propoxy)-dibenzothiophene-5-oxide;
2,8-bis-(3-piperidin-1-yl-propoxy)-dibenzothiophene-5-oxide;
2,8-bis-(2-trimethylammoniumethoxy)-dibenzothiophene-5-oxide;
2,8-bis-(3-trimethylammoniumpropoxy)-dibenzothiophene-5-oxide;
3,7-bis-(2-aminoethoxy)-dibenzothiophene-5,5-dioxide;
3,7-bis-(3-aminopropoxy)-dibenzothiophene-5,5-dioxide;
3,7-bis-(2-methylaminoethoxy)-dibenzothiophene-5,5-dioxide;
3,7-bis-(2-ethylaminoethoxy)-dibenzothiophene-5,5-dioxide;
3,7-bis-(2-n-propylaminoethoxy)-dibenzothiophene-5,5-dioxide;
3,7-bis-(3-methylaminopropoxy)-dibenzothiophene-5,5-dioxide;
3,7-bis-(3-ethylaminopropoxy)-dibenzothiophene-5,5-dioxide;
3,7-bis-(3-n-propylaminopropoxy)-dibenzothiophene-5,5-dioxide;
3,7-bis-(2-dimethylaminoethoxy)-dibenzothiophene-5,5-dioxide;
3,7-bis-(2-diethylaminoethoxy)-dibenzothiophene-5,5-dioxide;
3,7-bis-(2-di-n-propylaminoethoxy)-dibenzothiophene-5,5-dioxide;
3,7-bis-(3-dimethylaminopropoxy)-dibenzothiophene-5,5-dioxide;
3,7-bis-(3-diethylaminopropoxy)-dibenzothiophene-5,5-dioxide;
3,7-bis-(3-di-n-propylaminopropoxy)-dibenzothiophene-5,5-dioxide;
3,7-bis-(2-azetidin-1-yl-ethoxy)-dibenzothiophene-5,5-dioxide;
3,7-bis-(2-pyrrolidin-1-yl-ethoxy)-dibenzothiophene-5,5-dioxide;
3,7-bis-(2-piperidin-1-yl-ethoxy)-dibenzothiophene-5,5-dioxide;
3,7-bis-(3-azetidin-1-yl-propoxy)-dibenzothiophene-5,5-dioxide;
3,7-bis-(3-pyrrolidin-1-yl-propoxy)-dibenzothiophene-5,5-dioxide;
3,7-bis-(3-piperidin-1-yl-propoxy)-dibenzothiophene-5,5-dioxide;
3,7-bis-(2-trimethylammoniumethoxy)-dibenzothiophene-5,5-dioxide;
3,7-bis-(3-trimethylammoniumpropoxy)-dibenzothiophene-5,5-dioxide;
2,7-bis-(2-aminoethoxy)-dibenzothiophene-5,5-dioxide;
2,7-bis-(3-aminopropoxy)-dibenzothiophene-5,5-dioxide;
2,7-bis-(2-methylaminoethoxy)-dibenzothiophene-5,5-dioxide;
2,7-bis-(2-ethylaminoethoxy)-dibenzothiophene-5,5-dioxide;
2,7-bis-(2-n-propylaminoethoxy)-dibenzothiophene-5,5-dioxide;
2,7-bis-(3-methylaminopropoxy)-dibenzothiophene-5,5-dioxide;
2,7-bis-(3-ethylaminopropoxy)-dibenzothiophene-5,5-dioxide;
2,7-bis-(3-n-propylaminopropoxy)-dibenzothiophene-5,5-dioxide;
2,7-bis-(2-dimethylaminoethoxy)-dibenzothiophene-5,5-dioxide;
2,7-bis-(2-diethylaminoethoxy)-dibenzothiophene-5,5-dioxide;
2,7-bis-(2-di-n-propylaminoethoxy)-dibenzothiophene-5,5-dioxide;
2,7-bis-(3-dimethylaminopropoxy)-dibenzothiophene-5,5-dioxide;
2,7-bis-(3-diethylaminopropoxy)-dibenzothiophene-5,5-dioxide;
2,7-bis-(3-di-n-propylaminopropoxy)-dibenzothiophene-5,5-dioxide;
2,7-bis-(2-azetidin-1-yl-ethoxy)-dibenzothiophene-5,5-dioxide;
2,7-bis-(2-pyrrolidin-1-yl-ethoxy)-dibenzothiophene-5,5-dioxide;
2,7-bis-(2-piperidin-1-yl-ethoxy)-dibenzothiophene-5,5-dioxide;
2,7-bis-(3-azetidin-1-yl-propoxy)-dibenzothiophene-5,5-dioxide;
2,7-bis-(3-pyrrolidin-1-yl-propoxy)-dibenzothiophene-5,5-dioxide;
2,7-bis-(3-piperidin-1-yl-propoxy)-dibenzothiophene-5,5-dioxide;
2,7-bis-(2-trimethylammoniumethoxy)-dibenzothiophene-5,5-dioxide;
2,7-bis-(3-trimethylammoniumpropoxy)-dibenzothiophene-5,5-dioxide;
2,8-bis-(2-aminoethoxy)-dibenzothiophene-5,5-dioxide;
2,8-bis-(3-aminopropoxy)-dibenzothiophene-5,5-dioxide;
2,8-bis-(2-methylaminoethoxy)-dibenzothiophene-5,5-dioxide;
2,8-bis-(2-ethylaminoethoxy)-dibenzothiophene-5,5-dioxide;
2,8-bis-(2-n-propylaminoethoxy)-dibenzothiophene-5,5-dioxide;
2,8-bis-(3-methylaminopropoxy)-dibenzothiophene-5,5-dioxide;
2,8-bis-(3-ethylaminopropoxy)-dibenzothiophene-5,5-dioxide;
2,8-bis-(3-n-propylaminopropoxy)-dibenzothiophene-5,5-dioxide;
2,8-bis-(2-dimethylaminoethoxy)-dibenzothiophene-5,5-dioxide;
2,8-bis-(2-diethylaminoethoxy)-dibenzothiophene-5,5-dioxide;
2,8-bis-(2-di-n-propylaminoethoxy)-dibenzothiophene-5,5-dioxide;
2,8-bis-(3-dimethylaminopropoxy)-dibenzothiophene-5,5-dioxide;
2,8-bis-(3-diethylaminopropoxy)-dibenzothiophene-5,5-dioxide;
2,8-bis-(3-di-n-propylaminopropoxy)-dibenzothiophene-5,5-dioxide;
2,8-bis-(2-azetidin-1-yl-ethoxy)-dibenzothiophene-5,5-dioxide;
2,8-bis-(2-pyrrolidin-1-yl-ethoxy)-dibenzothiophene-5,5-dioxide;
2,8-bis-(2-piperidin-1-yl-ethoxy)-dibenzothiophene-5,5-dioxide;
2,8-bis-(3-azetidin-1-yl-propoxy)-dibenzothiophene-5,5-dioxide;
2,8-bis-(3-pyrrolidin 1-yl-propoxy)-dibenzothiophene-5,5-dioxide;
2,8-bis-(3-piperidin-1-yl-propoxy)-dibenzothiophene-5,5-dioxide;
2,8-bis-(2-trimethylammoniumethoxy)-dibenzothiophene-5,5-dioxide;
2,8-bis-(3-trimethylammoniumpropoxy)-dibenzothiophene-5,5-dioxide;
2,7-bis-(2-aminoethoxy)-9H-carbazole;
2,7-bis-(3-aminopropoxy)-9H-carbazole;
2,7-bis-(2-methylaminoethoxy)-9H-carbazole;
2,7-bis-(2-ethylaminoethoxy)-9H-carbazole;
2,7-bis-(2-n-propylaminoethoxy)-9H-carbazole;
2,7-bis-(3-methylaminopropoxy)-9H-carbazole;
2,7-bis-(3-ethylaminopropoxy)-9H-carbazole;
2,7-bis-(3-n-propylaminopropoxy)-9H-carbazole;
2,7-bis-(2-dimethylaminoethoxy)-9H-carbazole;
2,7-bis-(2-diethylaminoethoxy)-9H-carbazole;
2,7-bis-(2-di-n-propylaminoethoxy)-9H-carbazole;
2,7-bis-(3-dimethylaminopropoxy)-9H-carbazole;
2,7-bis-(3-diethylaminopropoxy)-9H-carbazole;
2,7-bis-(3-di-n-propylaminopropoxy)-9H-carbazole;
2,7-bis-(2-azetidin-1-yl-ethoxy)-9H-carbazole;
2,7-bis-(2-pyrrolidin-1-yl-ethoxy)-9H-carbazole;
2,7-bis-(2-piperidin-1-yl-ethoxy)-9H-carbazole;
2,7-bis-(3-azetidin-1-yl-propoxy)-9H-carbazole;
2,7-bis-(3-pyrrolidin-1-yl-propoxy)-9H-carbazole;
2,7-bis-(3-piperidin-1-yl-propoxy)-9H-carbazole;
2,7-bis-(2-trimethylammoniumethoxy)-9H-carbazole;
2,7-bis-(3-trimethylammoniumpropoxy)-9H-carbazole;
2,6-bis-(2-aminoethoxy)-9H-carbazole;
2,6-bis-(3-aminopropoxy)-9H-carbazole;
2,6-bis-(2-methylaminoethoxy)-9H-carbazole;
2,6-bis-(2-ethylaminoethoxy)-9H-carbazole;
2,6-bis-(2-n-propylaminoethoxy)-9H-carbazole;
2,6-bis-(3-methylaminopropoxy)-9H-carbazole;
2,6-bis-(3-ethylaminopropoxy)-9H-carbazole;
2,6-bis-(3-n-propylaminopropoxy)-9H-carbazole;
2,6-bis-(2-dimethylaminoethoxy)-9H-carbazole;
2,6-bis-(2-diethylaminoethoxy)-9H-carbazole;
2,6-bis-(2-di-n-propylaminoethoxy)-9H-carbazole;
2,6-bis-(3-dimethylaminopropoxy)-9H-carbazole;
2,6-bis-(3-diethylaminopropoxy)-9H-carbazole;
2,6-bis-(3-di-n-propylaminopropoxy)-9H-carbazole;
2,6-bis-(2-azetidin-1-yl-ethoxy)-9H-carbazole;
2,6-bis-(2-pyrrolidin-1-yl-ethoxy)-9H-carbazole;
2,6-bis-(2-piperidin-1-yl-ethoxy)-9H-carbazole;
2,6-bis-(3-azetidin-1-yl-propoxy)-9H-carbazole;
2,6-bis-(3-pyrrolidin-1-yl-propoxy)-9H-carbazole;
2,6-bis-(3-piperidin-1-yl-propoxy)-9H-carbazole;
2,6-bis-(2-trimethylammoniumethoxy)-9H-carbazole;
2,6-bis-(3-trimethylammoniumpropoxy)-9H-carbazole;
3,6-bis-(2-aminoethoxy)-9H-carbazole;
3,6-bis-(3-aminopropoxy)-9H-carbazole;
3,6-bis-(2-methylaminoethoxy)-9H-carbazole;
3,6-bis-(2-ethylaminoethoxy)-9H-carbazole;
3,6-bis-(2-n-propylaminoethoxy)-9H-carbazole;
3,6-bis-(3-methylaminopropoxy)-9H-carbazole;
3,6-bis-(3-ethylaminopropoxy)-9H-carbazole;
3,6-bis-(3-n-propylaminopropoxy)-9H-carbazole;
3,6-bis-(2-dimethylaminoethoxy)-9H-carbazole;
3,6-bis-(2-diethylaminoethoxy)-9H-carbazole;
3,6-bis-(2-di-n-propylaminoethoxy)-9H-carbazole;
3,6-bis-(3-dimethylaminopropoxy)-9H-carbazole;
3,6-bis-(3-diethylaminopropoxy)-9H-carbazole;
3,6-bis-(3-di-n-propylaminopropoxy)-9H-carbazole;
3,6-bis-(2-azetidin-1-yl-ethoxy)-9H-carbazole;
3,6-bis-(2-pyrrolidin-1-yl-ethoxy)-9H-carbazole;
3,6-bis-(2-piperidin-1-yl-ethoxy)-9H-carbazole;
3,6-bis-(3-azetidin-1-yl-propoxy)-9H-carbazole;
3,6-bis-(3-pyrrolidin-1-yl-propoxy)-9H-carbazole;
3,6-bis-(3-piperidin-1-yl-propoxy)-9H-carbazole;
3,6-bis-(2-trimethylammoniumethoxy)-9H-carbazole;
3,6-bis-(3-trimethylammoniumpropoxy)-9H-carbazole;
2,7-bis-(2-aminoethoxy)-9-methyl-9H-carbazole;
2,7-bis-(3-aminopropoxy)-9-methyl-9H-carbazole;
2,7-bis-(2-methylaminoethoxy)-9-methyl-9H-carbazole;
2,7-bis-(2-ethylaminoethoxy)-9-methyl-9H-carbazole;
2,7-bis-(2-n-propylaminoethoxy)-9-methyl-9H-carbazole;
2,7-bis-(3-methylaminopropoxy)-9-methyl-9H-carbazole;
2,7-bis-(3-ethylaminopropoxy)-9-methyl-9H-carbazole;
2,7-bis-(3-n-propylaminopropoxy)-9-methyl-9H-carbazole;
2,7-bis-(2-dimethylaminoethoxy)-9-methyl-9H-carbazole;
2,7-bis-(2-diethylaminoethoxy)-9-methyl-9H-carbazole;
2,7-bis-(2-di-n-propylaminoethoxy)-9-methyl-9H-carbazole;
2,7-bis-(3-dimethylaminopropoxy)-9-methyl-9H-carbazole;
2,7-bis-(3-diethylaminopropoxy)-9-methyl-9H-carbazole;
2,7-bis-(3-di-n-propylaminopropoxy)-9-methyl-9H-carbazole;
2,7-bis-(2-azetidin-1-yl-ethoxy)-9-methyl-9H-carbazole;
2,7-bis-(2-pyrrolidin-1-yl-ethoxy)-9-methyl-9H-carbazole;
2,7-bis-(2-piperidin-1-yl-ethoxy)-9-methyl-9H-carbazole;
2,7-bis-(3-azetidin-1-yl-propoxy)-9-methyl-9H-carbazole;
2,7-bis-(3-pyrrolidin-1-yl-propoxy)-9-methyl-9H-carbazole;
2,7-bis-(3-piperidin-1-yl-propoxy)-9-methyl-9H-carbazole;
2,7-bis-(2-trimethylammoniumethoxy)-9-methyl-9H-carbazole;
2,7-bis-(3-trimethylammoniumpropoxy)-9-methyl-9H-carbazole;
2,6-bis-(2-aminoethoxy)-9-methyl-9H-carbazole;
2,6-bis-(3-aminopropoxy)-9-methyl-9H-carbazole;
2,6-bis-(2-methylaminoethoxy)-9-methyl-9H-carbazole;
2,6-bis-(2-ethylaminoethoxy)-9-methyl-9H-carbazole;
2,6-bis-(2-n-propylaminoethoxy)-9-methyl-9H-carbazole;
2,6-bis-(3-methylaminopropoxy)-9-methyl-9H-carbazole;
2,6-bis-(3-ethylaminopropoxy)-9-methyl-9H-carbazole;
2,6-bis-(3-n-propylaminopropoxy)-9-methyl-9H-carbazole;
2,6-bis-(2-dimethylaminoethoxy)-9-methyl-9H-carbazole;
2,6-bis-(2-diethylaminoethoxy)-9-methyl-9H-carbazole;
2,6-bis-(2-di-n-propylaminoethoxy)-9-methyl-9H-carbazole;
2,6-bis-(3-dimethylaminopropoxy)-9-methyl-9H-carbazole;
2,6-bis-(3-diethylaminopropoxy)-9-methyl-9H-carbazole;
2,6-bis-(3-di-n-propylaminopropoxy)-9-methyl-9H-carbazole;
2,6-bis-(2-azetidin-1-yl-ethoxy)-9-methyl-9H-carbazole;
2,6-bis-(2-pyrrolidin-1-yl-ethoxy)-9-methyl-9H-carbazole;
2,6-bis-(2-piperidin-1-yl-ethoxy)-9-methyl-9H-carbazole;
2,6-bis-(3-azetidin-1-yl-propoxy)-9-methyl-9H-carbazole;
2,6-bis-(3-pyrrolidin-1-yl-propoxy)-9-methyl-9H-carbazole;
2,6-bis-(3-piperidin-1-yl-propoxy)-9-methyl-9H-carbazole;
2,6-bis-(2-trimethylammoniumethoxy)-9-methyl-9H-carbazole;
2,6-bis-(3-trimethylammoniumpropoxy)-9-methyl-9H-carbazole;
3,6-bis-(2-aminoethoxy)-9-methyl-9H-carbazole;
3,6-bis-(3-aminopropoxy)-9-methyl-9H-carbazole;
3,6-bis-(2-methylaminoethoxy)-9-methyl-9H-carbazole;
3,6-bis-(2-ethylaminoethoxy)-9-methyl-9H-carbazole;
3,6-bis-(2-n-propylaminoethoxy)-9-methyl-9H-carbazole;
3,6-bis-(3-methylaminopropoxy)-9-methyl-9H-carbazole;
3,6-bis-(3-ethylaminopropoxy)-9-methyl-9H-carbazole;
3,6-bis-(3-n-propylaminopropoxy)-9-methyl-9H-carbazole;
3,6-bis-(2-dimethylaminoethoxy)-9-methyl-9H-carbazole;
3,6-bis-(2-diethylaminoethoxy)-9-methyl-9H-carbazole;
3,6-bis-(2-di-n-propylaminoethoxy)-9-methyl-9H-carbazole;
3,6-bis-(3-dimethylaminopropoxy)-9-methyl-9H-carbazole;
3,6-bis-(3-diethylaminopropoxy)-9-methyl-9H-carbazole;
3,6-bis-(3-di-n-propylaminopropoxy)-9-methyl-9H-carbazole;
3,6-bis-(2-azetidin-1-yl-ethoxy)-9-methyl-9H-carbazole;
3,6-bis-(2-pyrrolidin-1-yl-ethoxy)-9-methyl-9H-carbazole;
3,6-bis-(2-piperidin-1-yl-ethoxy)-9-methyl-9H-carbazole;
3,6-bis-(3-azetidin-1-yl-propoxy)-9-methyl-9H-carbazole;
3,6-bis-(3-pyrrolidin-1-yl-propoxy)-9-methyl-9H-carbazole;
3,6-bis-(3-piperidin-1-yl-propoxy)-9-methyl-9H-carbazole;
3,6-bis-(2-trimethylammoniumethoxy)-9-methyl-9H-carbazole;
3,6-bis-(3-trimethylammoniumpropoxy)-9-methyl-9H-carbazole;
2,7-bis-(2-aminoethoxy)-xanthen-9-one;
2,7-bis-(3-aminopropoxy)-xanthen-9-one;
2,7-bis-(2-methylaminoethoxy)-xanthen-9-one;
2,7-bis-(2-ethylaminoethoxy)-xanthen-9-one;
2,7-bis-(2-n-propylaminoethoxy)-xanthen-9-one;
2,7-bis-(3-methylaminopropoxy)-xanthen-9-one;
2,7-bis-(3-ethylaminopropoxy)-xanthen-9-one;
2,7-bis-(3-n-propylaminopropoxy)-xanthen-9-one;
2,7-bis-(2-dimethylaminoethoxy)-xanthen-9-one;
2,7-bis-(2-diethylaminoethoxy)-xanthen-9-one;
2,7-bis-(2-di-n-propylaminoethoxy)-xanthen-9-one;
2,7-bis-(3-dimethylaminopropoxy)-xanthen-9-one;
2,7-bis-(3-diethylaminopropoxy)-xanthen-9-one;
2,7-bis-(3-di-n-propylaminopropoxy)-xanthen-9-one;
2,7-bis-(2-azetidin-1-yl-ethoxy)-xanthen-9-one;
2,7-bis-(2-pyrrolidin-1-yl-ethoxy)-xanthen-9-one;
2,7-bis-(2-piperidin-1-yl-ethoxy)-xanthen-9-one;
2,7-bis-(3-azetidin-1-yl-propoxy)-xanthen-9-one;
2,7-bis-(3-pyrrolidin-1-yl-propoxy)-xanthen-9-one;
2,7-bis-(3-piperidin-1-yl-propoxy)-xanthen-9-one;
2,7-bis-(2-trimethylammoniumethoxy)-xanthen-9-one;
2,7-bis-(3-trimethylammoniumpropoxy)-xanthen-9-one;
2,6-bis-(2-methylaminoethoxy)-xanthen-9-one;
2,6-bis-(2-ethylaminoethoxy)-xanthen-9-one;
2,6-bis-(2-n-propylaminoethoxy)-xanthen-9-one;
2,6-bis-(3-methylaminopropoxy)-xanthen-9-one;
2,6-bis-(3-ethylaminopropoxy)-xanthen-9-one;
2,6-bis-(3-n-propylaminopropoxy)-xanthen-9-one;
2,6-bis-(2-dimethylaminoethoxy)-xanthen-9-one;
2,6-bis-(2-diethylaminoethoxy)-xanthen-9-one;
2,6-bis-(2-di-n-propylaminoethoxy)-xanthen-9-one;
2,6-bis-(3-dimethylaminopropoxy)-xanthen-9-one;
2,6-bis-(3-diethylaminopropoxy)-xanthen-9-one;
2,6-bis-(3-di-n-propylaminopropoxy)-xanthen-9-one;
2,6-bis-(2-azetidin-1-yl-ethoxy)-xanthen-9-one;
2,6-bis-(2-pyrrolidin-1-yl-ethoxy)-xanthen-9-one;
2,6-bis-(2-piperidin-1-yl-ethoxy)-xanthen-9-one;
2,6-bis-(3-azetidin-1-yl-propoxy)-xanthen-9-one;
2,6-bis-(3-pyrrolidin-1-yl-propoxy)-xanthen-9-one;
2,6-bis-(3-piperidin-1-yl-propoxy)-xanthen-9-one;
2,6-bis-(2-trimethylammoniumethoxy)-xanthen-9-one;
2,6-bis-(3-trimethylammoniumpropoxy)-xanthen-9-one;
3,6-bis-(2-aminoethoxy)-xanthen-9-one;
3,6-bis-(3-aminopropoxy)-xanthen-9-one;
3,6-bis-(2-methylaminoethoxy)-xanthen-9-one;
3,6-bis-(2-ethylaminoethoxy)-xanthen-9-one;
3,6-bis-(2-n-propylaminoethoxy)-xanthen-9-one;
3,6-bis-(3-methylaminopropoxy)-xanthen-9-one;
3,6-bis-(3-ethylaminopropoxy)-xanthen-9-one;
3,6-bis-(3-n-propylaminopropoxy)-xanthen-9-one;
3,6-bis-(2-dimethylaminoethoxy)-xanthen-9-one;
3,6-bis-(2-diethylaminoethoxy)-xanthen-9-one;
3,6-bis-(2-di-n-propylaminoethoxy)-xanthen-9-one;
3,6-bis-(3-dimethylaminopropoxy)-xanthen-9-one;
3,6-bis-(3-diethylaminopropoxy)-xanthen-9-one;
3,6-bis-(3-di-n-propylaminopropoxy)-xanthen-9-one;
3,6-bis-(2-azetidin-1-yl-ethoxy)-xanthen-9-one;
3,6-bis-(2-pyrrolidinyl-ethoxy)-xanthen-9-one;
3,6-bis-(2-piperidin-1-yl-ethoxy)-xanthen-9-one;
3,6-bis-(3-azetidin-1-yl-propoxy)-xanthen-9-one;
3,6-bis-(3-pyrrolidin-1-yl-propoxy)-xanthen-9-one;
3,6-bis-(3-piperidin-1-yl-propoxy)-xanthen-9-one;
3,6-bis-(2-trimethylammoniumethoxy)-xanthen-9-one;
3,6-bis-(3-trimethylammoniumpropoxy)-xanthen-9-one;
2,7-bis-(2-aminoethoxy)-thioxanthen-9-one;
2,7-bis-(3-aminopropoxy)-thioxanthen-9-one;
2,7-bis-(2-methylaminoethoxy)-thioxanthen-9-one;
2,7-bis-(2-ethylaminoethoxy)-thioxanthen-9-one;
2,7-bis-(2-n-propylaminoethoxy)-thioxanthen-9-one;
2,7-bis-(3-methylaminopropoxy)-thioxanthen-9-one;
2,7-bis-(3-ethylaminopropoxy)-thioxanthen-9-one;
2,7-bis-(3-n-propylaminopropoxy)-thioxanthen-9-one;
2,7-bis-(2-dimethylaminoethoxy)-thioxanthen-9-one;
2,7-bis-(2-diethylaminoethoxy)-thioxanthen-9-one;
2,7-bis-(2-di-n-propylaminoethoxy)-thioxanthen-9-one;
2,7-bis-(3-dimethylaminopropoxy)-thioxanthen-9-one;
2,7-bis-(3-diethylaminopropoxy)-thioxanthen-9-one;
2,7-bis-(3-di-n-propylaminopropoxy)-thioxanthen-9-one;
2,7-bis-(2-azetidin-1-yl-ethoxy)-thioxanthen-9-one;
2,7-bis-(2-pyrrolidin-1-yl-ethoxy)-thioxanthen-9-one;
2,7-bis-(2-piperidin-1-yl-ethoxy)-thioxanthen-9-one;
2,7-bis-(3-azetidin-1-yl-propoxy)-thioxanthen-9-one;
2,7-bis-(3-pyrrolidin-1-yl-propoxy)-thioxanthen-9-one;
2,7-bis-(3-piperidin-1-yl-propoxy)-thioxanthen-9-one;
2,7-bis-(2-trimethylammoniumethoxy)-thioxanthen-9-one;
2,7-bis-(3-trimethylammoniumpropoxy)-thioxanthen-9-one;
2,6-bis-(2-methylaminoethoxy)-thioxanthen-9-one;
2,6-bis-(2-ethylaminoethoxy)-thioxanthen-9-one;
2,6-bis-(2-n-propylaminoethoxy)-thioxanthen-9-one;
2,6-bis-(3-methylaminopropoxy)-thioxanthen-9-one;
2,6-bis-(3-ethylaminopropoxy)-thioxanthen-9-one;
2,6-bis-(3-n-propylaminopropoxy)-thioxanthen-9-one;
2,6-bis-(2-dimethylaminoethoxy)-thioxanthen-9-one;
2,6-bis-(2-diethylaminoethoxy)-thioxanthen-9-one;
2,6-bis-(2-di-n-propylaminoethoxy)-thioxanthen-9-one;
2,6-bis-(3-dimethylaminopropoxy)-thioxanthen-9-one;
2,6-bis-(3-diethylaminopropoxy)-thioxanthen-9-one;
2,6-bis-(3-di-n-propylaminopropoxy)-thioxanthen-9-one;
2,6-bis-(2-azetidin-1-yl-ethoxy)-thioxanthen-9-one;
2,6-bis-(2-pyrrolidin-1-yl-ethoxy)-thioxanthen-9-one;
2,6-bis-(2-piperidin-1-yl-ethoxy)-thioxanthen-9-one;
2,6-bis-(3-azetidin-1-yl-propoxy)-thioxanthen-9-one;
2,6-bis-(3-pyrrolidin-1-yl-propoxy)-thioxanthen-9-one;
2,6-bis-(3-piperidin-1-yl-propoxy)-thioxanthen-9-one;
2,6-bis-(2-trimethylammoniumethoxy)-thioxanthen-9-one;
2,6-bis-(3-trimethylammoniumpropoxy)-thioxanthen-9-one;
3,6-bis-(2-aminoethoxy)-thioxanthen-9-one;
3,6-bis-(3-aminopropoxy)-thioxanthen-9-one;
3,6-bis-(2-methylaminoethoxy)-thioxanthen-9-one;
3,6-bis-(2-ethylaminoethoxy)-thioxanthen-9-one;
3,6-bis-(2-n-propylaminoethoxy)-thioxanthen-9-one;
3,6-bis-(3-methylaminopropoxy)-thioxanthen-9-one;
3,6-bis-(3-ethylaminopropoxy)-thioxanthen-9-one;
3,6-bis-(3-n-propylaminopropoxy)-thioxanthen-9-one;
3,6-bis-(2-dimethylaminoethoxy)-thioxanthen-9-one;
3,6-bis-(2-diethylaminoethoxy)-thioxanthen-9-one;
3,6-bis-(2-di-n-propylaminoethoxy)-thioxanthen-9-one;
3,6-bis-(3-dimethylaminopropoxy)-thioxanthen-9-one;
3,6-bis-(3-diethylaminopropoxy)-thioxanthen-9-one;
3,6-bis-(3-di-n-propylaminopropoxy)-thioxanthen-9-one;
3,6-bis-(2-azetidin-1-yl-ethoxy)-thioxanthen-9-one;
3,6-bis-(2-pyrrolidin-1-yl-ethoxy)-thioxanthen-9-one;
3,6-bis-(2-piperidin-1-yl-ethoxy)-thioxanthen-9-one;
3,6-bis-(3-azetidin-1-yl-propoxy)-thioxanthen-9-one;
3,6-bis-(3-pyrrolidin-1-yl-propoxy)-thioxanthen-9-one;
3,6-bis-(3-piperidin-1-yl-propoxy)-thioxanthen-9-one;
3,6-bis-(2-trimethylammoniumethoxy)-thioxanthen-9-one;
3,6-bis-(3-trimethylammoniumpropoxy)-thioxanthen-9-one;
2,7-bis-(2-aminoethoxy)-10H-acridine-9-one;
2,7-bis-(3-aminopropoxy)-10H-acridine-9-one;
2,7-bis-(2-methylaminoethoxy)-10H-acridine-9-one;
2,7-bis-(2-ethylaminoethoxy)-10H-acridine-9-one;
2,7-bis-(2-n-propylaminoethoxy)-10H-acridine-9-one;
2,7-bis-(3-methylaminopropoxy)-10H-acridine-9-one;
2,7-bis-(3-ethylaminopropoxy)-10H-acridine-9-one;
2,7-bis-(3-n-propylaminopropoxy)-10H-acridine-9-one;
2,7-bis-(2-dimethylaminoethoxy)-10H-acridine-9-one;
2,7-bis-(2-diethylaminoethoxy)-10H-acridine-9-one;
2,7-bis-(2-di-n-propylaminoethoxy)-10H-acridine-9-one;
2,7-bis-(3-dimethylaminopropoxy)-10H-acridine-9-one;
2,7-bis-(3-diethylaminopropoxy)-10H-acridine-9-one;
2,7-bis-(3-di-n-propylaminopropoxy)-10H-acridine-9-one;
2,7-bis-(2-azetidin-1-yl-ethoxy)-10H-acridine-9-one;
2,7-bis-(2-pyrrolidin-1-yl-ethoxy)-10H-acridine-9-one;
2,7-bis-(2-piperidin-1-yl-ethoxy)-10H-acridine-9-one;
2,7-bis-(3-azetidin-1-yl-propoxy)-10H-acridine-9-one;
2,7-bis-(3-pyrrolidin-1-yl-propoxy)-10H-acridine-9-one;
2,7-bis-(3-piperidin-1-yl-propoxy)-10H-acridine-9-one;
2,7-bis-(2-trimethylammoniumethoxy)-10H-acridine-9-one;
2,7-bis-(3-trimethylammoniumpropoxy)-10H-acridine-9-one;
2,6-bis-(2-methylaminoethoxy)-10H-acridine-9-one;
2,6-bis-(2-ethylaminoethoxy)-10H-acridine-9-one;
2,6-bis-(2-n-propylaminoethoxy)-10H-acridine-9-one;
2,6-bis-(3-methylaminopropoxy)-10H-acridine-9-one;
2,6-bis-(3-ethylaminopropoxy)-10H-acridine-9-one;
2,6-bis-(3-n-propylaminopropoxy)-10H-acridine-9-one;
2,6-bis-(2-dimethylaminoethoxy)-10H-acridine-9-one;
2,6-bis-(2-diethylaminoethoxy)-10H-acridine-9-one;
2,6-bis-(2-di-n-propylaminoethoxy)-10H-acridine-9-one;
2,6-bis-(3-dimethylaminopropoxy)-10H-acridine-9-one;
2,6-bis-(3-diethylaminopropoxy)-10H-acridine-9-one;
2,6-bis-(3-di-n-propylaminopropoxy)-10H-acridine-9-one;
2,6-bis-(2-azetidin-1-yl-ethoxy)-10H-acridine-9-one;
2,6-bis-(2-pyrrolidin-1-yl-ethoxy)-10H-acridine-9-one;
2,6-bis-(2-piperidin-1-yl-ethoxy)-10H-acridine-9-one;
2,6-bis-(3-azetidin-1-yl-propoxy)-10H-acridine-9-one;
2,6-bis-(3-pyrrolidin-1-yl-propoxy)-10H-acridine-9-one;
2,6-bis-(3-piperidin-1-yl-propoxy)-10H-acridine-9-one;
2,6-bis-(2-trimethylammoniumethoxy)-10H-acridine-9-one;
2,6-bis-(3-trimethylammoniumpropoxy)-10H-acridine-9-one;
3,6-bis-(2-aminoethoxy)-10H-acridine-9-one;
3,6-bis-(3-aminopropoxy)-10H-acridine-9-one;
3,6-bis-(2-methylaminoethoxy)-10H-acridine-9-one;
3,6-bis-(2-ethylaminoethoxy)-10H-acridine-9-one;
3,6-bis-(2-n-propylaminoethoxy)-10H-acridine-9-one;
3,6-bis-(3-methylaminopropoxy)-10H-acridine-9-one;
3,6-bis-(3-ethylaminopropoxy)-10H-acridine-9-one;
3,6-bis-(3-n-propylaminopropoxy)-10H-acridine-9-one;
3,6-bis-(2-dimethylaminoethoxy)-10H-acridine-9-one;
3,6-bis-(2-diethylaminoethoxy)-10H-acridine-9-one;
3,6-bis-(2-di-n-propylaminoethoxy)-10H-acridine-9-one;
3,6-bis-(3-dimethylaminopropoxy)-10H-acridine-9-one;
3,6-bis-(3-diethylaminopropoxy-10H-acridine-9-one;
3,6-bis-(3-di-n-propylaminopropoxy)-10H-acridine-9-one;
3,6-bis-(2-azetidin-1-yl-ethoxy)-10H-acridine-9-one;
3,6-bis-(2-pyrrolidin-1-yl-ethoxy)-10H-acridine-9-one;
3,6-bis-(2-piperidin-1-yl-ethoxy)-10H-acridine-9-one;
3,6-bis-(3-azetidin-1-yl-propoxy)-10H-acridine-9-one;
3,6-bis-(3-pyrrolidin-1-yl-propoxy)-10H-acridine-9-one;
3,6-bis-(3-piperidin-1-yl-propoxy)-10H-acridine-9-one;
3,6-bis-(2-trimethylammoniumethoxy)-10H-acridine-9-one;
3,6-bis-(3-trimethylammoniumpropoxy)-10H-acridine-9-one;
2,7-bis-(2-aminoethoxy)-10-methyl-10H-acridine-9-one;
2,7-bis-(3-aminopropoxy)-10-methyl-10H-acridine-9-one;
2,7-bis-(2-methylaminoethoxy)-10-methyl-10H-acridine-9-one;
2,7-bis-(2-ethylaminoethoxy)-10-methyl-10H-acridine-9-one;
2,7-bis-(2-n-propylaminoethoxy)-10-methyl-10H-acridine-9-one;
2,7-bis-(3-methylaminopropoxy)-10-methyl-10H-acridine-9-one;
2,7-bis-(3-ethylaminopropoxy)-10-methyl-10H-acridine-9-one;
2,7-bis-(3-n-propylaminopropoxy)-10-methyl-10H-acridine-9-one;
2,7-bis-(2-dimethylaminoethoxy)-10-methyl-10H-acridine-9-one;
2,7-bis-(2-diethylaminoethoxy)-10-methyl-10H-acridine-9-one;
2,7-bis-(2-di-n-propylaminoethoxy)-10-methyl-10H-acridine-9-one;
2,7-bis-(3-dimethylaminopropoxy)-10-methyl-10H-acridine-9-one;
2,7-bis-(3-diethylaminopropoxy)-10-methyl-10H-acridine-9-one;
2,7-bis-(3-di-n-propylaminopropoxy)-10-methyl-10H-acridine-9-one;
2,7-bis-(2-azetidin-1-yl-ethoxy)-10-methyl-10H-acridine-9-one;
2,7-bis-(2-pyrrolidin-1-yl-ethoxy)-10-methyl-10H-acridine-9-one;
2,7-bis-(2-piperidin-1-yl-ethoxy)-10-methyl-10H-acridine-9-one;
2,7-bis-(3-azetidin-1-yl-propoxy)-10-methyl-10H-acridine-9-one;
2,7-bis-(3-pyrrolidin-1-yl-propoxy)-10-methyl-10H-acridine-9-one;
2,7-bis-(3-piperidin-1-yl-propoxy)-10-methyl-10H-acridine-9-one;
2,7-bis-(2-trimethylammoniumethoxy)-10-methyl-10H-acridine-9-one;
2,7-bis-(3-trimethylammoniumpropoxy)-10-methyl-10H-acridine-9-one;
2,6-bis-(2-methylaminoethoxy)-10-methyl-10H-acridine-9-one;
2,6-bis-(2-ethylaminoethoxy)-10-methyl-10H-acridine-9-one;
2,6-bis-(2-n-propylaminoethoxy)-10-methyl-10H-acridine-9-one;
2,6-bis-(3-methylaminopropoxy)-10-methyl-10H-acridine-9-one;
2,6-bis-(3-ethylaminopropoxy)-10-methyl-10H-acridine-9-one;
2,6-bis-(3-n-propylaminopropoxy)-10-methyl-10H-acridine-9-one;
2,6-bis-(2-dimethylaminoethoxy)-10-methyl-10H-acridine-9-one;
2,6-bis-(2-diethylaminoethoxy)-10-methyl-10H-acridine-9-one;
2,6-bis-(2-di-n-propylaminoethoxy)-10-methyl-10H-acridine-9-one;
2,6-bis-(3-dimethylaminopropoxy)-10-methyl-10H-acridine-9-one;
2,6-bis-(3-diethylaminopropoxy 10-methyl-10H-acridine-9-one;
2,6-bis-(3-di-n-propylaminopropoxy)-10-methyl-10H-acridine-9-one;
2,6-bis-(2-azetidin-1-yl-ethoxy)-10-methyl-10H-acridine-9-one;
2,6-bis-(2-pyrrolidin-1-yl-ethoxy)-10-methyl-10H-acridine-9-one;
2,6-bis-(2-piperidin-1-yl-ethoxy)-10-methyl-10H-acridine-9-one;
2,6-bis-(3-azetidin-1-yl-propoxy)-10-methyl-10H-acridine-9-one;
2,6-bis-(3-pyrrolidin-1-yl-propoxy)-10-methyl-10H-acridine-9-one;
2,6-bis-(3-piperidin-1-yl-propoxy)-10-methyl-10H-acridine-9-one;
2,6-bis-(2-trimethylammoniumethoxy)-10-methyl-10H-acridine-9-one;
2,6-bis-(3-trimethylammoniumpropoxy)-10-methyl-10H-acridine-9-one;
3,6-bis-(2-aminoethoxy)-10-methyl-10H-acridine-9-one;
3,6-bis-(3-aminopropoxy)-10-methyl-10H-acridine-9-one;
3,6-bis-(2-methylaminoethoxy)-10-methyl-10H-acridine-9-one;
3,6-bis-(2-ethylaminoethoxy)-10-methyl-10H-acridine-9-one;
3,6-bis-(2-n-propylaminoethoxy)-10-methyl-10H-acridine-9-one;
3,6-bis-(3-methylaminopropoxy)-10-methyl-10H-acridine-9-one;
3,6-bis-(3-ethylaminopropoxy)-10-methyl-10H-acridine-9-one;
3,6-bis-(3-n-propylaminopropoxy)-10-methyl-10H-acridine-9-one;
3,6-bis-(2-dimethylaminoethoxy)-10-methyl-10H-acridine-9-one;
3,6-bis-(2-diethylaminoethoxy)-10-methyl-10H-acridine-9-one;
3,6-bis-(2-di-n-propylaminoethoxy)-10-methyl-10H-acridine-9-one;
3,6-bis-(3-dimethylaminopropoxy)-10-methyl-10H-acridine-9-one;
3,6-bis-(3-diethylaminopropoxy)-10-methyl-10H-acridine-9-one;
3,6-bis-(3-di-n-propylaminopropoxy)-10-methyl-10-acridine-9-one;
3,6-bis-(2-azetidin-1-yl-ethoxy)-10-methyl-10H-acridine-9-one;
3,6-bis-(2-pyrrolidin-1-yl-ethoxy)-10-methyl-10H-acridine-9-one;
3,6-bis-(2-piperidin-1-yl-ethoxy)-10-methyl-10H-acridine-9-one;
3,6-bis-(3-azetidin-1-yl-propoxy)-10-methyl-10H-acridine-9-one;
3,6-bis-(3-pyrrolidin-1-yl-propoxy)-10-methyl-10H-acridine-9-one;
3,6-bis-(3-piperidin-1-yl-propoxy)-10-methyl-10H-acridine-9-one;
3,6-bis-(2-trimethylammoniumethoxy)-10-methyl-10H-acridine-9-one;
3,6-bis-(3-trimethylammoniumpropoxy)-10-methyl-10H-acridine-9-one;
3,8-bis-(2-aminoethoxy)-5H-phenanthridin-6-one;
3,8-bis-(3-aminopropoxy)-5H-phenanthridin-6-one;
3,8-bis-(2-methylaminoethoxy)-5H-phenanthridin-6-one;
3,8-bis-(2-ethylaminoethoxy)-5H-phenanthridin-6-one;
3,8-bis-(2-n-propylaminoethoxy)-5H-phenanthridin-6-one;
3,8-bis-(3-methylaminopropoxy)-5H-phenanthridin-6-one;
3,8-bis-(3-ethylaminopropoxy)-5H-phenanthridin 6-one;
3,8-bis-(3-n-propylaminopropoxy)-5H-phenanthridin 6-one;
3,8-bis-(2-dimethylaminoethoxy)-5H-phenanthridin-6-one;
3,8-bis-(2-diethylaminoethoxy)-5H-phenanthridin-6-one;
3,8-bis-(2-di-n-propylaminoethoxy)-5H-phenanthridin-6-one;
3,8-bis-(3-dimethylaminopropoxy)-5H-phenanthridin-6-one;
3,8-bis-(3-diethylaminopropoxy)-5H-phenanthridin-6-one;
3,8-bis-(3-di-n-propylaminopropoxy)-5H-phenanthridin-6-one;
3,8-bis-(2-azetidin-1-yl-ethoxy)-5H-phenanthridin-6-one;
3,8-bis-(2-pyrrolidin-1-yl-ethoxy)-5H-phenanthridin-6-one;
3,8-bis-(2-piperidin-1-yl-ethoxy)-5H-phenanthridin-6-one;
3,8-bis-(3-azetidin-1-yl-propoxy)-5H-phenanthridin-6-one;
3,8-bis-(3-pyrrolidin-1-yl-propoxy)-5H-phenanthridin-6-one;
3,8-bis-(3-piperidin-1-yl-propoxy)-5H-phenanthridin-6-one;
3,8-bis-(2-trimethylammoniumethoxy)-5H-phenanthridin-6-one;
3,8-bis-(3-trimethylammoniumpropoxy)-5H-phenanthridin-6-one;
3,8-bis-(2-aminoethoxy)-5-methyl-5H-phenanthridin-6-one;
3,8-bis-(3-aminopropoxy)-5-methyl-5H-phenanthridin-6-one;
3,8-bis-(2-methylaminoethoxy)-5-methyl-5H-phenanthridin-6-one;
3,8-bis-(2-ethylaminoethoxy)-5-methyl-5H-phenanthridin-6-one;
3,8-bis-(2-n-propylaminoethoxy)-5-methyl-5H-phenanthridin-6-one;
3,8-bis-(3-methylaminopropoxy)-5-methyl-5H-phenanthridin-6-one;
3,8-bis-(3-ethylaminopropoxy)-5-methyl-5H-phenanthridin-6-one;
3,8-bis-(3-n-propylaminopropoxy)-5-methyl-5H-phenanthridin-6-one;
3,8-bis-(2-dimethylaminoethoxy)-5-methyl-5H-phenanthridin-6-one;
3,8-bis-(2-diethylaminoethoxy)-5-methyl-5H-phenanthridin-6-one;
3,8-bis-(2-di-n-propylaminoethoxy)-5-methyl-5H-phenanthridin-one;
3,8-bis-(3-dimethylaminopropoxy)-5-methyl-5H-phenanthridin-6-one;
3,8-bis-(3-diethylaminopropoxy)-5-methyl-5H-phenanthridin-6-one;
3,8-bis-(3-di-n-propylaminopropoxy)-5-methyl-5H-phenanthridin-6-one;
3,8-bis-(2-azetidin-1-yl-ethoxy)-5-methyl-5H-phenanthridin-6-one;
3,8-bis-(2-pyrrolidin-1-yl-ethoxy)-5-methyl-5H-phenanthridin-6-one;
3,8-bis-(2-piperidin-1-yl-ethoxy)-5-methyl-5H-phenanthridin-6-one;
3,8-bis-(3-azetidin-1-yl-propoxy)-5-methyl-5H-phenanthridin-6-one;
3,8-bis-(3-pyrrolidin 1-yl-propoxy)-5-methyl-5H-phenanthridin-6-one;
3,8-bis-(3-piperidin-1-yl-propoxy)-5-methyl-5H-phenanthridin-6-one;
3,8-bis-(2-trimethylammoniumethoxy)-5-methyl-5H-phenanthridin-6-one;
3,8-bis-(3-trimethylammoniumpropoxy)-5-methyl-5H-phenanthridin-6-one;
3,8-bis-(2-aminoethoxy)-benzo[c]chromen-6-one;
3,8-bis-(3-aminopropoxy)-benzo[c]chromen-6-one;
3,8-bis-(2-methylaminoethoxy)-benzo[c]chromen-6-one;
3,8-bis-(2-ethylaminoethoxy)-benzo[c]chromen-6-one;
3,8-bis-(2-n-propylaminoethoxy)-benzo[c]chromen-6-one;
3,8-bis-(3-methylaminopropoxy)-benzo[c]chromen-6-one;
3,8-bis-(3-ethylaminopropoxy)-benzo[c]chromen-6-one;
3,8-bis-(3-n-propylaminopropoxy)-benzo[c]chromen-6-one;
3,8-bis-(2-dimethylaminoethoxy)-benzo[c]chromen-6-one;
3,8-bis-(2-diethylaminoethoxy)-benzo[c]chromen-6-one;
3,8-bis-(2-di-n-propylaminoethoxy)-benzo[c]chromen-6-one;
3,8-bis-(3-dimethylaminopropoxy)-benzo[c]chromen-6-one;
3,8-bis-(3-diethylaminopropoxy)-benzo[c]chromen-6-one;
3,8-bis-(3-di-n-propylaminopropoxy)-benzo[c]chromen-6-one;
3,8-bis-(2-azetidin-1-yl-ethoxy)-benzo[c]chromen-6-one;
3,8-bis-(2-pyrrolidin-1-yl-ethoxy)-benzo[c]chromen-6-one;
3,8-bis-(2-piperidin-1-yl-ethoxy)-benzo[c]chromen-6-one;
3,8-bis-(3-azetidin-1-yl-propoxy)-benzo[c]chromen-6-one;
3,8-bis-(3-pyrrolidin-1-yl-propoxy)-benzo[c]chromen-6-one;
3,8-bis-(3-piperidin-1-yl-propoxy)-benzo[c]chromen-6-one;
3,8-bis-(2-trimethylammoniumethoxy)-benzo[c]chromen-6-one;
3,8-bis-(3-trimethylammoniumpropoxy)-benzo[c]chromen-6-one;
2,7-bis-(2-aminoethoxy)-10H-phenanthren-9-one;
2,7-bis-(3-aminopropoxy)-10H-phenanthren-9-one;
2,7-bis-(2-methylaminoethoxy)-10H-phenanthren-9-one;
2,7-bis-(2-ethylaminoethoxy)-10H-phenanthren-9-one;
2,7-bis-(2-n-propylaminoethoxy)-10H-phenanthren-9-one;
2,7-bis-(3-methylaminopropoxy)-10H-phenanthren-9-one;
2,7-bis-(3-ethylaminopropoxy)-10H-phenanthren-9-one;
2,7-bis-(3-n-propylaminopropoxy)-10H-phenanthren-9-one;
2,7-bis-(2-dimethylaminoethoxy)-10H-phenanthren-9-one;
2,7-bis-(2-diethylaminoethoxy)-10H-phenanthren-9-one;
2,7-bis-(2-di-n-propylaminoethoxy)-10H-phenanthren-9-one;
2,7-bis-(3-dimethylaminopropoxy)-10H-phenanthren-9-one;
2,7-bis-(3-diethylaminopropoxy)-10H-phenanthren-9-one;
2,7-bis-(3-di-n-propylaminopropoxy)-10H-phenanthren-9-one;
2,7-bis-(2-azetidin-1-yl-ethoxy)-10H-phenanthren-9-one;
2,7-bis-(2-pyrrolidin-1-yl-ethoxy)-10H-phenanthren-9-one;
2,7-bis-(2-piperidin-1-yl-ethoxy)-10H-phenanthren-9-one;
2,7-bis-(3-azetidin-1-yl-propoxy)-110H-phenanthren-9-one;
2,7-bis-(3-pyrrolidin-1-yl-propoxy)-10H-phenanthren-9-one;
2,7-bis-(3-piperidin-1-yl-propoxy)-10H-phenanthren-9-one;
2,7-bis-(2-trimethylammoniumethoxy)-10H-phenanthren-9-one;
2,7-bis-(3-trimethylammoniumpropoxy)-10H-phenanthren-9-one;
   and pharmaceutically acceptable salts, esters, amides, and prodrugs thereof. References that may be useful in preparing the compounds are Sill, A. D., et al., J. Med. Chem., 1973, 16, 3, 240-245; Andrews, E. R., et al., J. Med. Chem., 1974, 17, 8, 882-886; Albrecht, W. L., et al., J. Med. Chem., 1974, 17, 8, 886-890; Grisar, J. M., et al., J. Med. Chem., 1974, 17, 8, 890-893; Carr, A. A., et al., J. Med. Chem., 1975, 19, 9, 1142-1148; and Albrecht, W. L., et al., J. Med. Chem, 1977, 20, 3, 364-371.

For example, α7 nAChRs have been shown to play a significant role in enhancing cognitive function, including aspects of learning, memory and attention (Levin, E. D., J. Neurobiol. 53: 633-640, 2002). As such, α7 ligands are suitable for the treatment of conditions and disorders related to memory and/or cognition including, for example, attention deficit disorder, attention deficit hyperactivity disorder (ADHD), Alzheimer's disease (AD), mild cognitive impairment, senile dementia, AIDS dementia, Pick's Disease, dementia associated with Lewy bodies, and dementia associated with Down's syndrome, as well as cognitive deficits associated with schizophrenia.

In addition, α7-containing nAChRs have been shown to be involved in the cytoprotective effects of nicotine both in vitro (Jonnala, R. B. and Buccafusco, J. J., J. Neurosci. Res. 66: 565-572, 2001) and in vivo (Shimohama, S. et al., Brain Res. 779: 359-363, 1998). More particularly, neurodegeneration underlies several progressive CNS disorders, including, but not limited to, Alzheimer's disease, Parkinson's disease, amyotrophic lateral sclerosis, Huntington's disease, dementia with Lewy bodies, as well as diminished CNS function resulting from traumatic brain injury. For example, the impaired function of α7 nAChRs by β-amyloid peptides linked to Alzheimer's disease has been implicated as a key factor in development of the cognitive deficits associated with the disease (Liu, Q.-S., Kawai, H., Berg, D. K., PNAS 98: 4734-4739, 2001). The activation of α7 nAChRs has been shown to block this neurotoxicity (Kihara, T. et al., J. Biol. Chem. 276: 13541-13546, 2001). As such, selective ligands that enhance α7 activity can counter the deficits of Alzheimer's and other neurodegenerative diseases.

Alpha-7 nAChRs also have been implicated in aspects of neurodevelopment, for example neurogenesis of the brain. (Falk, L. et al., Developmental Brain Research 142:151-160, 2003; Tsuneki, H., et al., J. Physiol. (London) 547:169-179, 2003; Adams, C. E., et al., Developmental Brain Research 139:175-187, 2002). As such, α7 nAChRs can be useful in preventing or treating conditions or disorders associated with impaired neurodevelopment, for example schizophrenia. (Sawa A., Mol. Med. 9:3-9, 2003).

Schizophrenia is a complex disease that is characterized by abnormalities in perception, cognition, and emotions. Significant evidence implicates the involvement of α7 nAChRs in this disease, including a measured deficit of these receptors in post-mortem patients (Sawa A., Mol. Med. 9:3-9, 2003; Leonard, S. Eur. J. Pharmacol. 393: 237-242, 2000). Deficits in sensory processing (gating) are one of the hallmarks of schizophrenia. These deficits can be normalized by nicotinic ligands that operate at the α7 nAChR (Adler L. E. et al., Schizophrenia Bull. 24: 189-202, 1998; Stevens, K. E. et al., Psychopharmacology 136: 320-327, 1998). Thus, α7 ligands demonstrate potential in the treatment schizophrenia.

Angiogenesis, a process involved in the growth of new blood vessels, is important in beneficial systemic functions, such as wound healing, vascularization of skin grafts, and enhancement of circulation, for example, increased circulation around a vascular occlusion. Non-selective nAChR agonists like nicotine have been shown to stimulate angiogenesis (Heeschen, C. et al., Nature Medicine 7: 833-839, 2001). Improved angiogenesis has been shown to involve activation of the α7 nAChR (Heeschen, C. et al, J. Clin. Invest. 110: 527-536, 2002). For example, improved conditions related to inflammation, ischemia, cardiac ischemia, and wound healing, for example in diabetic persons, have been associated with α7 nAChR activity (Jacobi, J., et al., Am. J. Pathol. 161:97-104, 2002). Therefore, nAChR ligands that are selective for the α7 subtype offer improved potential for stimulating angiogenesis with an improved side effect profile.

A population of α7 nAChRs in the spinal cord modulate serotonergic transmission that have been associated with the pain-relieving effects of nicotinic compounds (Cordero-Erausquin, M. and Changeux, J.-P. PNAS 98:2803-2807, 2001). The α7 nAChR ligands demonstrate therapeutic potential for the treatment of pain states, including acute pain, post-surgical pain, as well as chronic pain states including inflammatory pain and neuropathic pain. Moreover, α7 nAChRs are expressed on the surface of primary macrophages that are involved in the inflammation respore, and that activation of the α7 receptor inhibits release of TNF and other cytokines that trigger the inflammation response (Wang, H. et al Nature 421: 384-388, 2003). Therefore, selective α7 ligands demonstrate potential for treating conditions involving inflammation and pain.

The mammalian sperm acrosome reaction is an exocytosis process important in fertilization of the ovum by sperm. Activation of an α7 nAChR on the sperm cell has been shown to be essential for the acrosome reaction (Son, J. H. and Meizel, S. Biol. Reproduct. 68: 1348-1353 2003). Consequently, selective α7 agents demonstrate utility for treating fertility disorders.

Compounds of the invention are particularly useful for treating and preventing a condition or disorder affecting memory, cognition, neurodegeneration, neurodevelopment, and schizophrenia.

Cognitive impairment associated with schizophrenia often limits the ability of patients to function normally, a symptom not adequately treated by commonly available treatments, for example, treatment with an atypical antipsychotic. (Rowley, M. et al., J. Med. Chem. 44: 477-501, 2001). Such cognitive deficit has been linked to dysfunction of the nicotinic cholinergic system, in particular with decreased activity at α7 receptors. (Friedman, J. I. et al., Biol Psychiatry, 51: 349-357, 2002). Thus, activators of α7 receptors can provide useful treatment for enhancing cognitive function in schizophrenic patients who are being treated with atypical antipsychotics. Accordingly, the combination of an α7 nAChR ligand and an atypical antipsychotic would offer improved therapeutic utility. Specific examples of suitable atypical antipsychotics include, but are not limited to, clozapine, risperidone, olanzapine, quietapine, ziprasidone, zotepine, iloperidone, and the like.

Actual dosage levels of active ingredients in the pharmaceutical compositions of this invention can be varied so as to obtain an amount of the active compound(s) that is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

When used in the above or other treatments, a therapeutically effective amount of one of the compounds of the invention can be employed in pure form or, where such forms exist, in pharmaceutically acceptable salt, ester, amide or prodrug form. Alternatively, the compound can be administered as a pharmaceutical composition containing the compound of interest in combination with one or more pharmaceutically acceptable carriers. The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors wellknown in the medical arts. For example, it is well within the skill of the art to start doses of the compound at levels lower than required to achieve the desired therapeutic effect and to gradually increase the dosage until the desired effect is achieved.

The total daily dose of the compounds of this invention administered to a human or lower animal range from about 0.10 μg/kg body weight to about 10 mg/kg body weight. More preferable doses can be in the range of from about 0.10 μg/kg body weight to about 1 mg/kg body weight. If desired, the effective daily dose can be divided into multiple doses for purposes of administration. Consequently, single dose compositions may contain such amounts or submultiples thereof to make up the daily dose.

Methods for Preparing Compounds of the Invention

As used in the descriptions of the schemes and the examples, certain abbreviations are intended to have the following meanings: Ac for acetyl; Bu for a butyl; Bn for benzyl; cat. for cataylst; dba for dibenzylidene acetone; DMF for dimethyl formamide; EtOH for ethanol; Et₃N for triethylamine; EtOAc for ethyl acetate; HPLC for high pressure liquid chromatography; ⁱPr for isopropyl; ⁱPrOAc for isopropyl acetate; LAH for lithium aluminum hydride; Me for methyl; MeOH for methanol; NBS for N-bromosuccinimide; NMP for N-methylpyrrolidine; OAc for acetoxy; ONF for nonaflate or —OSO₂CF₂CF₂CF₂CF₃; Pd/C for palladium on carbon; Ph for phenyl; Rh/C for rhodium on carbon; tBu for tert-butyl; ^{t}BuO for tert-butoxide; and THF for tetrahydrofuran.

The reactions exemplified in the schemes are performed in a solvent appropriate to the reagents and materials employed and suitable for the transformations being effected. The described transformations may require modifying the order of the synthetic steps or selecting one particular process scheme over another in order to obtain a desired compound of the invention, depending on the functionality present on the molecule.

Nitrogen protecting groups can be used for protecting amine groups present in the described compounds. Such methods, and some suitable nitrogen protecting groups, are described in Greene and Wuts (Protective Groups In Organic Synthesis, Wiley and Sons, 1999). For example, suitable nitrogen protecting groups include, but are not limited to, tert-butoxycarbonyl (Boc), benzyloxycarbonyl (Cbz), benzyl (Bn), acetyl, and trifluoroacetyl. More particularly, the BOC protecting group may be removed by treatment with an acid such as trifluoroacetic acid or hydrochloric acid. The Cbz and Bn protecting groups may be removed by catalytic hydrogenation. The acetyl and trifluoroacetyl protecting groups may be removed by a hydroxide ion.

The methods described below can entail use of various enantiomers. Where the stereochemistry is shown in the Schemes, it is intended for illustrative purposes only.

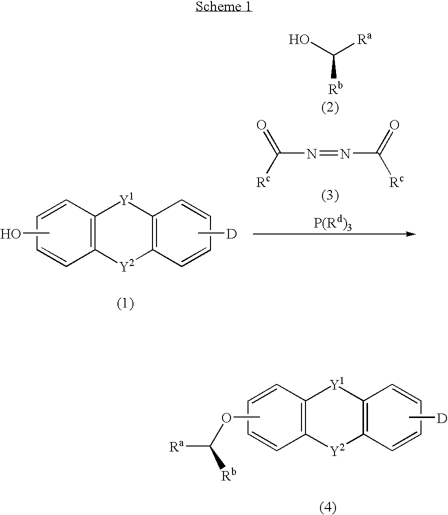

Compounds of formula (4), wherein Y¹ and Y² are as defined for a compound of formula (I) or (II), D is as defined for group A or B and R^{a} and R^{b} are hydrogen, alkyl, heterocyclealkyl, or form a cyclic group of formula as shown for group (a), (b), or (c) for compounds of formula (I) or (II), can be prepared as shown in Scheme 1. A hydroxylated tricyclic core is treated under standard Mitsunobu reaction conditions with a desired alcohol (2) using a dialkyl azodicarboxylate reagent (3), wherein R^{c} is alkoxy or alkylamino, and a reagent of the formula P(R^{d})₃ wherein R^{d} is phenyl or butyl, as described in the art to provide compounds of formula (4). Suitable conditions for the reaction are further described in Hughes, D. L., Org. React., 1992, 42, 335; Tusonda, T., et al., Tetrahedron Lett., 1993, 34, 1639; and Tunoori, A. R., et al., Tetrahedron Lett., 1998, 39, 8751.

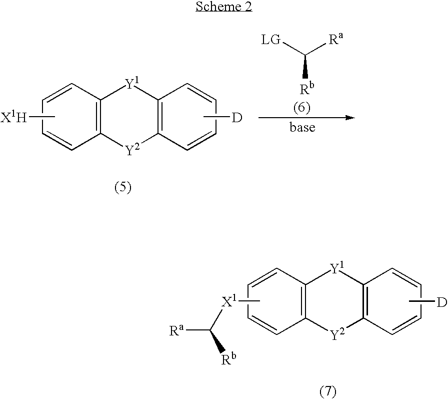

Compounds of formula (7), wherein Y¹ and Y² are as defined for a compound of formula (I) or (II), D is as defined for group A or B and R^{a} and R^{b} are hydrogen, alkyl, heterocyclealkyl, or form a cyclic group of formula as shown for group (a), (b), or (c) for compounds of formula (I) or (II), can be prepared as shown in Scheme 2. A substituted tricyclic core of formula (5), wherein X¹ is O, S, or —NH—, is reacted with an alkylating reagent of formula (6), wherein LG represents a halide, methanesulfonate, toluensulfonate, or triflate group, in the presence of a base, to provide compounds of formula (7).

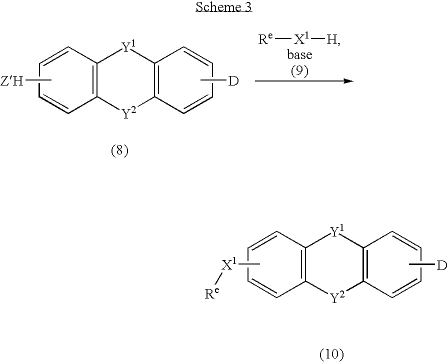

Compounds of formula (10), wherein Y¹ and Y² are as defined for a compound of formula (I) or (II), D is as defined for group A or B and —X¹—Re is a group of formula (a), (b), (c), (d), (e), (e, or (g) for compounds of formula (I) or (II), can be prepared as shown in Scheme 3. A substituted tricyclic core of formula (6), wherein Z′ is a halide, such as bromine, chlorine, fluorine, and iodine, triflate, or nitro, is reacted with a reagent of formula (9), wherein X¹ is O, S, or —NH—, in the presence of a base, to provide compounds of formula (10). Suitable conditions for the reaction are further described in U.S. Pat. No. 6,379,590.

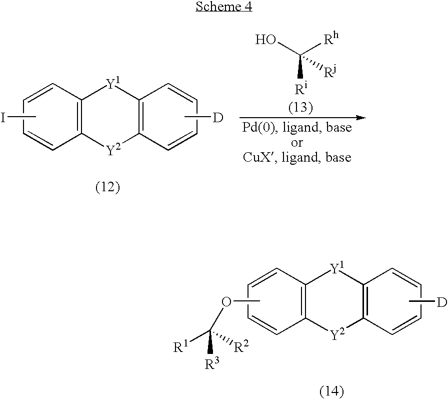

Compounds of formula (14), wherein Y¹ and Y² are as defined for compounds of formula (I) or (II), D is as defined for a group A or B of formula (I) or (II), and R^{h}, R^{k} and R^{J} form a cyclic or acyclic group as defined for a group of formula (a), (b), or (c) in compounds of formula (I) or (II), can be prepared as shown in Scheme 4. An iodinated tricyclic compound of formula (12), wherein Y¹, Y², and D are as previously defined for compounds of formula (14), treated with a substituted alcohol of formula (13), wherein R^{h}, Rⁱ, and R^{j} are as described for compounds of formula (14), in the presence of a palladium catalyst and a ligand, for example a phosphine ligand, in the presence of a base. Alternatively, the reaction can be carried out by treating the compound of formula (12) with the alcohol reagent of formula (13) in the presence of a copper catalyst, CuX′, for example a copper halide, including copper bromide, copper chloride, copper fluoride, and copper iodide, with a ligand in the presence of base. Suitable conditions for the reaction are further described in Muci, A. R., et al., Topics Current Chem., 2002, 219, 131, and Ley, S. V., et al., Angew. Chem. Int. Ed., 2003, 42, 5400.

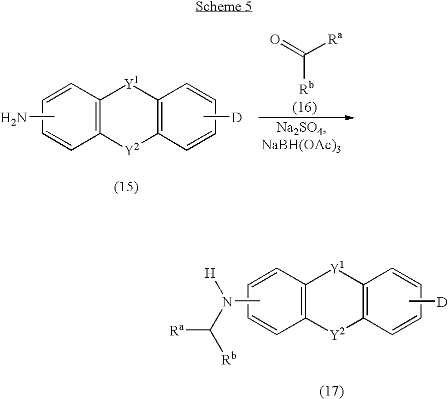

Compounds of the formula (17), wherein Y¹ and Y² are as defined for compounds of formula (I) or (II), D is as defined for A or B in a compound of formula (I) or (II), and R^{a} and R^{b} form the cyclic or acyclic moiety of group (a), (b), or (c) for a compound of formula (I) or (II), prepared as shown in Scheme 5. An amine substituted tricyclic compound of formula (15), wherein Y¹, Y², and D are as described for compounds of formula (17) can be treated with a ketone of formula (16), wherein R^{a} and R^{b} are as defined for compounds of formula (I) or (Il), in Na₂SO₄ and sodium triacetoxy borohydride, NaBH(OAc)₃. Suitable conditions for the reaction are further described in Coe, J., et al., Tetrahedron Lett., 1996, 37, 6045.

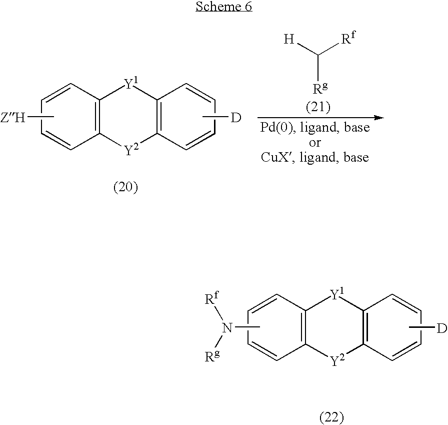

Compounds of formula (22), wherein Y¹ and Y² are as defined for compounds of formula (I) or (II), D is as defined for a group A or B as defined for compounds of formula (I) or (II), and R^{f} and R^{g} form a cyclic moiety of group (d), (e), or (g) as defined for compounds of formula (I) or (II), can be prepared as shown in Scheme 6. A substituted tricyclic starting material of formula (20), wherein Z″ is chloride, bromide, iodide, trifluoroacetate, or ONF, i.e. nonaflate or —OSO₂CF₂CF₂CF₂CF₃ as described in J. Org. Chem., 2003, 68(25), 9563-9573, can be reacted with an amine reagent of formula (21), wherein R^{f} and R^{g} are as defined for a compound of formula (20), to in the presence of a palladium (0) catalyst, and a ligand, for example a phosphine ligand, in the presence of base to provide a compound of formula (22). Alternatively, the compound of formula (20) can be reacted with a copper catalyst, CuX′, for example a copper halide, including copper bromide, copper chloride, copper fluoride, and copper iodide, in the presence of base to provide a compound of formula (22). Suitable conditions for the reaction are further described in Muci, A. R., et al., Topics Current Chem., 2002, 219, 131, and Ley, S. V., et al., Angew. Chem. Int. Ed., 2003, 42, 5400.

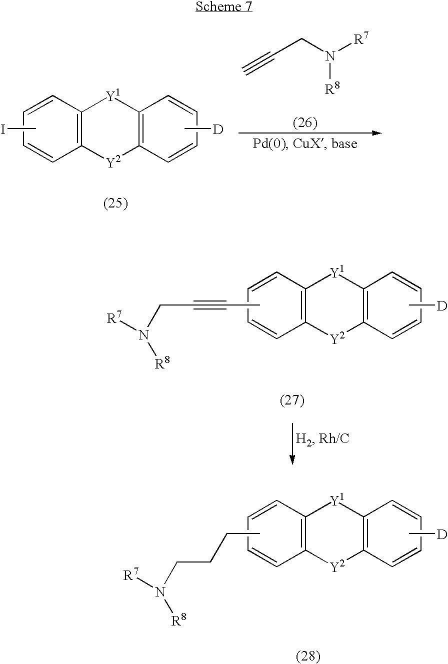

Compounds of formulas (27) and (28), wherein Y¹ and Y² are as defined for compounds of formula (I) or (II), D is as defined for a group A or B as defined for compounds of formula (I) or (II), and R⁷ and R⁸ are as defined for compounds of formula (I) or (II), can be prepared according to Scheme 7. Iodinated tricyclic compounds of formula (25), wherein Y¹, Y², and D are as defined for compounds of formula (28), can be treated with a propargyl amine reagent of formula (26), wherein Ft and R⁷ are as defined for compounds of formula (I) or (II), in the presence of a palladium(0) catalyst, and a copper halide, CuX′, including for example copper bromide, copper chloride, copper fluoride, and copper iodide, in the presence of a base to provide compounds of formula (27). Compounds of formula (27) can be reduced by hydrogenation using a rhodium catalyst on carbon to provide compounds of formula (28).

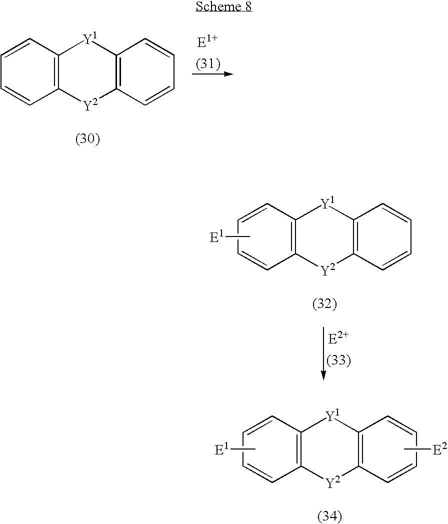

Compounds of formula (34), wherein Y¹ and Y² are as defined for compounds of formula (I) or (II), E¹ and E² are as defined for R^{x}, or a group A or B, as defined for compounds of formula (I) or (II), can be prepared according to Scheme 8. Unsubstituted tricyclic compounds of formula (30), wherein Y¹ and Y² are as defined for compounds of formula (34), can be treated with an electrophile of formula (31), for example, iodide, bromide, chloride, nitro, acetyl, or SOS, to provide compounds of formula (32). Compounds of formula (32) can be further be treated with a second electrophile of formula (33), which is as defined for compounds of formula (31) and can be either the same or different, to provide compounds of formula (34).

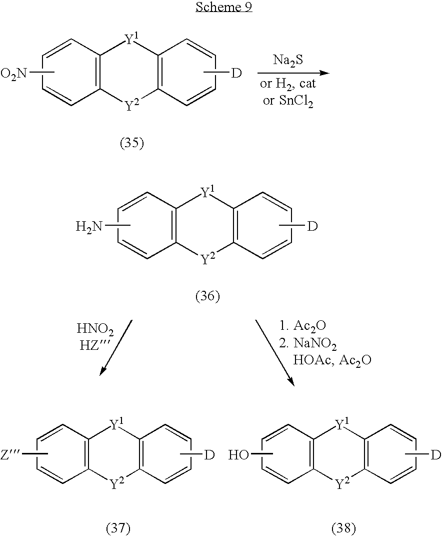

Compounds of formulas (37) and (38), wherein Y¹ and Y² are as defined for compounds of formula (I) or (II), D as defined for R^{x}, or a group A or B, as defined for compounds of formula (I) or (II), and Z′″ is bromide, chloride, fluoride, iodide, and hydroxy, can be prepared according to Scheme 9. Nitro-substitutedtricyclic compounds of formula (35), wherein Y¹, Y², and D are as defined for compounds of formulas (37) and (38) can be reduced by treatment sodium sulfide, catalytic hydrogenation, or treatment with tin chloride, to provide amine-substituted compounds of formula (36). Compounds of formula (36) can be reacted with HNO₂ and an acid of a suitable halide, or water, to provide compounds of formula (37). Alternatively, compounds of formula (36) can be reacted with Ac₂O followed by NaNo₂, acetic acid, and Ac₂O to provide hydroxylated compounds of formula (38). Suitable conditions for the reactions are further described in Perry, P. J., et al., J. Med. Chem., 1999, 42, 2679; Burke, M., et al., Synth. Commun., 1976, 6, 371; and Glatzhofer, D. T., et al., Org. Lett., 2002, 4, 2349.

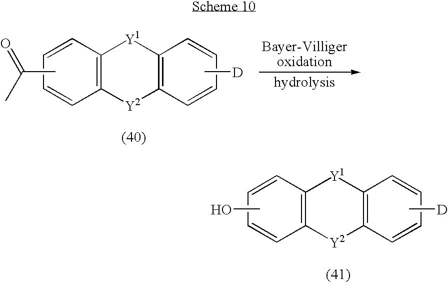

Compounds of formula (41), wherein Y¹ and Y² are as defined for compounds of formula (I) or (II), and D as defined for R^{x}, or a group A or B, as defined for compounds of formula (I) or (II), can be prepared according to Scheme 10. Tricyclic methyl ketone compounds of formula (40), wherein Y¹, Y², and D are as defined for compounds of formulas (I) or (II), can be oxidized under standard Bayer-Villiger oxidation conditions, followed by hydrolysis, to provide compounds of formula (41). Suitable conditions for the reactions are further described in Burke, M., et al., Synth. Commun., 1976, 6, 371.

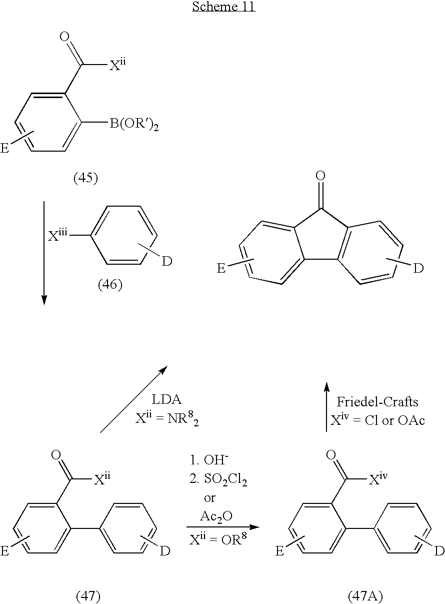

Compounds of formula (48), wherein Y¹ is —C(═O), Y² is a bond, and D and E are as defined for a group A or B for compounds of formula (I) or (II), can be prepared according to Scheme 11. Boronic acids of aryl amides of formula (45), wherein E is as defined for a group A or B for compounds of formula (I) or (II), are coupled with aryl halides of formula (46), wherein Xⁱⁱⁱ is a halide, including chloride, bromide, and iodide, or trifluoromethanesulfonyl, and D is as defined for a group A or B for compounds of formula (I) or (II), under standard Suzuki coupling reactions, for example a palladium catalyst and a ligand in the presence of base, to provide a compound of formula (47), wherein Xⁱⁱ is OR⁸ or NR⁸₂, wherein R⁸ is hydrogen or alkyl. A compound of formula (47), wherein Xⁱⁱ is OR⁸ can be transformed into a compound of formula (47A), wherein X^{iv} is chloride, by treatment with hydroxide followed in a second step by thionyl chloride, or into compounds of formula 47(A), wherein X^{iv} is acetoxy, by treatment with hydroxide followed in a second step by acetic anhydride. Compounds of formula (47A) can be treated under Friedel-Crafts conditions when X^{iv} is chloride or acetoxy to provide a compound of formula (48). Compounds of formula (47) can be treated with lithium diisopropylamide (LDA) when Xⁱⁱ is NR⁸₂ to provide a compound of formula (48). Suitable conditions for the reactions are further described in Ciske, F. L., et al., Synthesis, 1998, 1195; Fu, J., et al., J. Org. Chem., 1991, 56, 1683; Kym, P. R., et al., J. Med. Chem., 1996, 39, 4897.

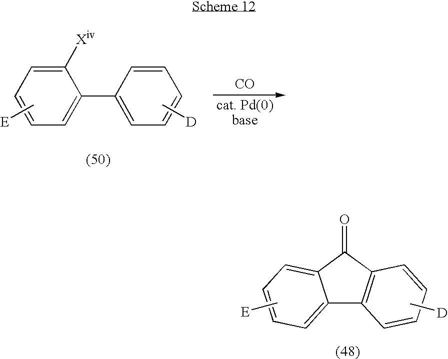

Compounds of formulas (48), wherein Y¹ is —C(═O), Y² is a bond, and D and E are as defined for a group A or B for compounds of formula (I) or (II), also can be prepared according to Scheme 12. A compound of formula (50), wherein X^{iv} is bromide or iodide and D and E are as defined for compounds of formula (48), can be treated with carbon monoxide in the presence of a palladium(0) catalyst and base to provide compounds of formula (48). Suitable conditions for the reaction are further described in Campo, M. A., et al., J. Org. Chem., 2002, 67, 5616.

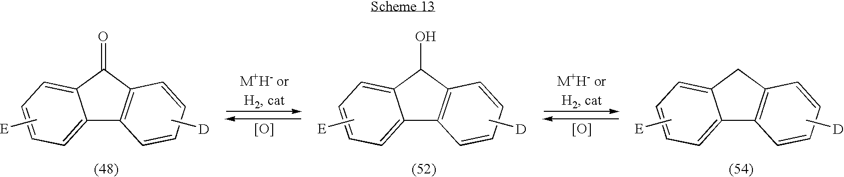

Compounds of formula (54), wherein Y¹ is —CH₂—, Y² is a bond, and D and E are as defined for group A or B in a compound of formula (I) or (II) can be prepared as shown in Scheme 13. The ketone group of (48) can be reduced by using a metal hydride or via hydrogenation to provide the hydroxy group of (52), which can be further reduced by the same methods to provide the methylene group of (54). Compounds of formula (54) can be converted to compounds of formula (52) by standard oxidation conditions and further converted to compounds of formula (48) by standard oxidation conditions as well. Suitable conditions for the reactions are further described in Ting, P. C., et al., Bioorg. Med. Chem. Left., 2002, 12, 2643, and Burke, M., et al., Synth. Commun., 1976,6, 371.

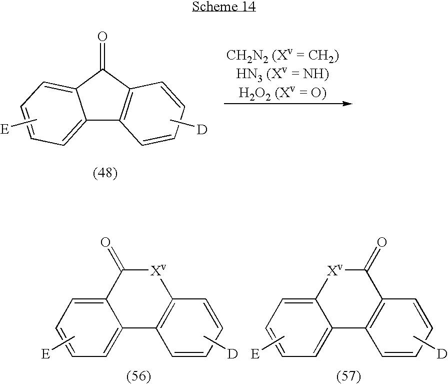

Compounds of formulas (56) and (57), wherein Y¹ is —X^{v}—C(═O)—, X^{v} is as shown in Scheme 14 above, Y² is a bond, and D and E are as defined for group A or B in a compound of formula (I) or (II) can be prepared as shown in Scheme 14. Compounds of formula (48) can be reacted with CH₂N₂, HN₃, and H₂O₂ to provide the respective compounds of formulas (56) and (57). Suitable conditions for the reactions are further described in U.S. Pat. Nos. 4,169,897; 3,838,131; 3,838,134; 3,932,643; and 4,059,702.

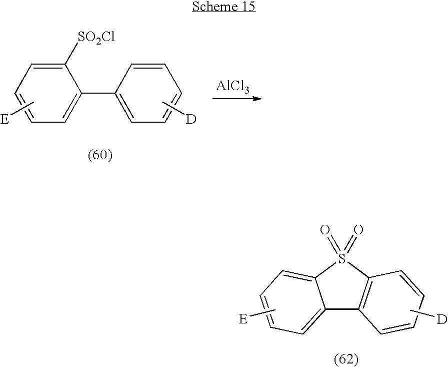

Compounds of formula (62), wherein Y¹ is —S(O)₂—, Y² is a bond, and D and E are as defined for group A or B for compounds of formulas (I) or (II), can be prepared according to Scheme 15. Biphenyl sulfonylchloride compounds of formula (60) are treated with aluminum chloride to provide compounds of formula (62). Suitable conditions for the reaction are further described in Davies, W., et al., J. Chem. Soc. Abst., 1995, 1565.

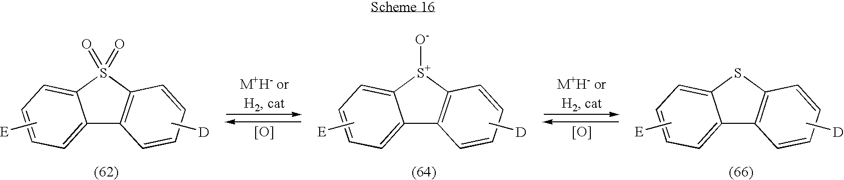

Compounds of formula (66), wherein Y¹ is —S—, Y² is a bond, and D and E are as defined for group A or B in a compound of formula (I) or (II) can be prepared as shown in Scheme 16. The sulfonyl group of (62) can be reduced by using a metal halide or via hydrogenation to provide compounds of formula (64), which can be further reduced by the same methods to provide compounds of formula (66). Compounds of formula (66) can be converted to compounds of formula (64) by standard oxidation conditions and further converted to compounds of formula (62) by standard oxidation conditions as well.

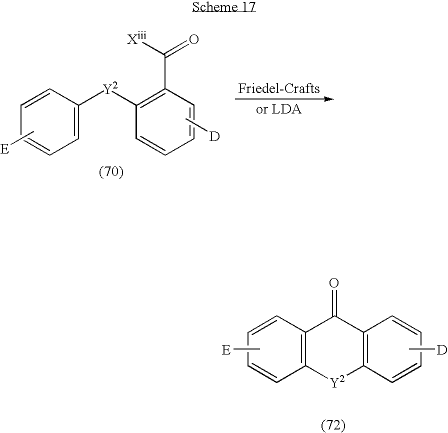

Compounds of formula (72), wherein Y¹ is —C(═O), Y² is as defined for compounds of formulas (I) or (II), and D and E are as defined for group A or B for compounds of formula (I) or (II), can be prepared as shown in Scheme 17. Compounds of formula (70), wherein Xⁱⁱⁱ is chloride, acetoxy, or NR′₂, wherein R′ is hydrogen or alkyl, and Y², D, and E are as defined for compounds of formula (72) can be treated under Friedel-Crafts conditions when Xⁱⁱⁱ is chloride or acetoxy, or with lithium diisopropylamide (LDA) when Xⁱⁱⁱ is a NR′₂, to provide a compound of formula (72). Suitable conditions for the reactions are further described in Familioni, O. B., et al., Synlett., 1997,1081; Gobbi, S., et al., J. Med. Chem, 2002, 45, 4931; and Olah, G. A., et al., Synlett., 1999, 7, 1067.

The compounds and intermediates of the invention may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

The compounds of the invention have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, carbonic, fumaric, gluconic, acetic, propionic, salicylic, hydrochloric, hydrobromic, phosphoric, sulfuric, citric, or hydroxybutyric acid, camphorsulfonic, malic, phenylacetic, aspartic, glutamic, and the like.

Compositions of the Invention

The invention also provides pharmaceutical compositions comprising a therapeutically effective amount of a compound of formula (I) in combination with a pharmaceutically acceptable carrier. The compositions comprise compounds of the invention formulated together with one or more non-toxic pharmaceutically acceptable carriers. The pharmaceutical compositions can be formulated for oral administration in solid or liquid form, for parenteral injection or for rectal administration.

The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other nontoxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of one skilled in the art of formulations.

The pharmaceutical compositions of this invention can be administered to humans and other mammals orally, rectally, parenterally, intracistemally, intravaginally, intraperitoneally, topically (as by powders, ointments or drops), bucally or as an oral or nasal spray. The term “parenterally,” as used herein, refers to modes of administration, including intravenous, intramuscular, intraperitoneal, intrasternal, subcutaneous, intraarticular injection and infusion.

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like, and suitable mixtures thereof, vegetable oils (such as olive oil) and injectable organic esters such as ethyl oleate, or suitable mixtures thereof. Suitable fluidity of the composition may be maintained, for example, by the use of a coating such as lecithin, by the maintenance of the required particle size in the case of dispersions, and by the use of surfactants.

These compositions can also contain adjuvants such as preservative agents, wetting agents, emulsifying agents, and dispersing agents. Prevention of the action of microorganisms can be ensured by various antibacterial and antifungal agents, for example, parabens, chlorobutanol, phenol, sorbic acid, and the like. It also can be desirable to include isotonic agents, for example, sugars, sodium chloride and the like. Prolonged absorption of the injectable pharmaceutical form can be brought about by the use of agents delaying absorption, for example, aluminum monostearate and gelatin.

In some cases, in order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug can depend upon its rate of dissolution, which, in turn, may depend upon crystal size and crystalline form. Alternatively, a parenterally administered drug form can be administered by dissolving or suspending the drug in an oil vehicle.

Suspensions, in addition to the active compounds, can contain suspending agents, for example, ethoxylated isostearyl alcohols, polyoxyethylene sorbitol and sorbitan esters, microcrystalline cellulose, aluminum metahydroxide, bentonite, agaragar, tragacanth, and mixtures thereof.

If desired, and for more effective distribution, the compounds of the invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations also are prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions can be formulated according to the known art using suitable dispersing or wetting agents and suspending agents. The sterile injectable preparation also can be a sterile injectable solution, suspension or emulsion in a nontoxic, parenterally acceptable diluent or solvent such as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, one or more compounds of the invention is mixed with at least one inert pharmaceutically acceptable carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearete, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols.

The solid dosage forms of tablets, dragees, capsules, pills, and granules can be prepared with coatings and shells such as enteric coatings and other coatings well-known in the pharmaceutical formulating art. They can optionally contain opacifying agents and can also be of a composition that they release the active ingredient(s) only, or preferentially, in a certain part of the intestinal tract in a delayed manner. Examples of materials useful for delaying release of the active agent can include polymeric substances and waxes.

Compositions for rectal or vaginal administration are preferably suppositories which can be prepared by mixing the compounds of this invention with suitable non-irritating carriers such as cocoa butter, polyethylene glycol or a suppository wax which are solid at ambient temperature but liquid at body temperature and therefore melt in the rectum or vaginal cavity and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. A desired compound of the invention is admixed under sterile conditions with a pharmaceutically acceptable carrier and any needed preservatives or buffers as may be required. Ophthalmic formulation, eardrops, eye ointments, powders and solutions are also contemplated as being within the scope of this invention.

The ointments, pastes, creams and gels may contain, in addition to an active compound of this invention, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, lactose, talc, silicic acid, aluminum hydroxide, calcium silicates and polyamide powder, or mixtures of these substances. Sprays can additionally contain customary propellants such as chlorofluorohydrocarbons.

Compounds of the invention also can be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the invention, stabilizers, preservatives, and the like. The preferred lipids are the natural and synthetic phospholipids and phosphatidylcholines (lecithins) used separately or together.

Methods to form liposomes are known in the art. See, for example, Prescott, Ed., Methods in Cell Biology, Volume XIV, Academic Press, New York, N.Y., (1976), p 33 et seq.

Dosage forms for topical administration of a compound of this invention include powders, sprays, ointments and inhalants. The active compound is mixed under sterile conditions with a pharmaceutically acceptable carrier and any needed preservatives, buffers or propellants. Ophthalmic formulations, eye ointments, powders and solutions are also contemplated as being within the scope of this invention. Aqueous liquid compositions of the invention also are particularly useful.

The compounds of the invention can be used in the form of pharmaceutically acceptable salts, esters, or amides derived from inorganic or organic acids. The term “pharmaceutically acceptable salts, esters and amides,” as used herein, include salts, zwitterions, esters and amides of compounds of formula (I) which are, within the scope of sound medical judgment, suitable for use in contact with the tissues of humans and lower animals without undue toxicity, irritation, allergic response, and the like, are commensurate with a reasonable benefit/risk ratio, and are effective for their intended use.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. The salts can be prepared in situ during the final isolation and purification of the compounds of the invention or separately by reacting a free base function with a suitable organic acid.

Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persolfate, 3-phenylpropionate, picrate, pivalate, propionate, succinate, tartrate, thiocyanate, phosphate, glutamate, bicarbonate, p-toluenesulfonate and undecanoate.

Also, the basic nitrogen-containing groups can be quaternized with such agents as lower alkyl halides such as methyl, ethyl, propyl, and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; arylalkyl halides such as benzyl and phenethyl bromides and others. Water or oil soluble or dispersible products are thereby obtained.

Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid, and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylamine, dimethylamine, trimethylamine, triethylamine, diethylamine, ethylamine and the such as. Other representative organic amines useful for the formation of base addition salts include ethylenediamine, ethanolamine, diethanolamine, piperidine, and piperazine.

The term “pharmaceutically acceptable ester,” as used herein, refers to esters of compounds of the invention which hydrolyze in vivo and include those that break down readily in the human body to leave the parent compound or a salt thereof. Examples of pharmaceutically acceptable, ncn-toxic esters of the invention include C₁-to-C₆ alkyl esters and C₅-to-C₇ cycloalkyl esters, although C₁-to-C₄ alkyl esters are preferred. Esters of the compounds of formula (I) can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, alkyl trifilate, for example with methyl iodide, benzyl iodide, cyclopentyl iodide. They also can be prepared by reaction of the compound with an acid such as hydrochloric acid and an alkylcarboxylic acid such as acetic acid, or with acid and an arylcarboxylic acid such as benzoic acid.

The term “pharmaceutically acceptable amide,” as used herein, refers to non-toxic amides of the invention derived from ammonia, primary C₁-to-C₆ alkyl amines and secondary C₁-to-C₆ dialkyl amines. In the case of secondary amines, the amine can also be in the form of a 5- or 6-membered heterocycle containing one nitrogen atom. Amides derived from ammonia, C₁-to-C₃ alkyl primary amides and C₁-to-C₂ dialkyl secondary amides are preferred. Amides of the compounds of formula (I) can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable esters are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, piperidine. They also can be prepared by reaction of the compound with an acid such as sulfuric acid and an alkylcarboxylic acid such as acetic acid, or with acid and an arylcarboxylic acid such as benzoic acid under dehydrating conditions as with molecular sieves added. The composition can contain a compound of the invention in the form of a pharmaceutically acceptable prodrug.

The term “pharmaceutically acceptable prodrug” or “prodrug,” as used herein, represents those prodrugs of the compounds of the invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the invention can be rapidly transformed in vivo to a parent compound of formula (I), for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

The invention contemplates pharmaceutically active compounds either chemically synthesized or formed by in vivo biotransformation to compounds of formula (I).

The compounds of the invention and processes for making compounds for the method of the invention will be better understood by reference to the following Examples, which are intended as an illustration of and not a limitation upon the scope of the invention.

### EXAMPLES

### Example 1

### 2,7-Bis[(2R)-1-methylpyrrolidin-2-ylmethoxy]-fluoren-9-one di-p-toluenesulfonate

### Example 1A

### 2,7-Bis[(2R)-1-Boc-pyrrolidin-2-ylmethoxy]-fluoren-9-one

To a solution of 2,7-dihydroxyfluoren-9-one (0.21 g, 1.0 mmol; see Synth. Commun. 1976, 6, 371) and (2R)-(+)-1-Boc-2-pyrrolidinemethanol (0.81 g, 4.0 mmol; Aldrich) in dry THF (10 mL) was added polymer-bound triphenylphosphine (1.3 g, 4.0 mmol; Aldrich) followed by di-tert-butylazodicarboxylate (920 mg, 4.00 mmol; Aldrich). The mixture was stirred overnight (16 h) at room temperature, then filtered through diatomaceous earth, rinsing with ethyl acetate. After concentrating the solution, the residue was purified by flash chromatography (35 g silica gel, 10-30% ethyl acetate-hexane) to afford the title compound (310 mg, 0.54 mmol; 54%). MS (DCl/NH₃): m/z 596 (M+18)⁺.

### Example 1B

### 2,7-Bis[(2R)-pyrrolidin-2-ylmethoxy]-fluoren-9-one

The product of Example 1A (310 mg, 0.54 mmol) in CH₂Cl₂ (9 mL) was treated with trifluoroacetic acid (3 mL; EM Science) as described in Example 11B, and was purified by flash chromatography (35 g silica gel, eluting with 5-10% of 10% NH₄OH/MeOH in CH₂Cl₂) to afford the title compound (180 mg, 0.48 mmol, 89% yield). MS (DCl/NH₃): m/z 379 (M+H)⁺.

### Example 1C

### 2,7-Bis[(2R)-1-methylpyrrolidin-2-ylmethoxy]-fluoren-9-one

The product of Example 1B (180 mg, 0.48 mmol) was dissolved in dry DMF (5 mL), cooled to 0° C. in an ice bath and treated with 60% sodium hydride (60 mg, 1.4 mmol; Aldrich), followed by iodomethane (0.06 mL, 0.94 mmol, Baker). The mixture was allowed to warm to ambient temperature and stirred for 16 hours, then poured onto ice and extracted with ethyl acetate. The combined organic phases were washed with brine (25 mL), dried over MgSO₄, concentrated under reduced pressure, and purified by flash chromatography (35 g silica gel, 1:5:94 NH₄OH—MeOH—CH₂Cl₂) to afford the title compound (50 mg, 0.12 mmol, 26% yield).

### Example 1D

### 2,7-Bis[(2R)-1-methylpyrrolidin-2-ylmethoxy]-fluoren-9-one di-p-toluenesulfonate

The product of Example 1C (50 mg, 0.12 mmol) was dissolved in ethyl acetate (5 mL) and ethanol (0.2 mL), then p-toluenesulfonic acid monohydrate (46 mg, 0.24 mmol; Aldrich) was added. After stirring the mixture for 16 hours, the resulting solid was collected by filtration to afford the title compound (68 mg, 0.09 mmol; 74%): ¹H NMR (300 MHz, methanol-d4): δ 7.68 (4H, d, J=8 Hz), 7.54 (2H, d, J=8 Hz), 7.27-7.14 (8H, m), 4.46 (2H, dd, J=11, 3 Hz), 4.27 (2H, dd, J=11, 7 Hz), 3.88 (2H, m), 3.73 (2H, m), 3.35-3.19 (4H, m), 3.07 (6H, s), 2.48-1.97 (12H, m). MS (DCl/NH₃): m/z 407 (M+1)⁺. Anal. Calcd. for C₂₅H₃₀N₂O₃.2C₇H₈O₃S.H₂O: C, 60.92; H, 6.29; N, 3.64. Found: C, 61.02; H, 6.25; N, 3.57.

### Example 2

### 2,7-Bis[(2R)-azetidin-2-ylmethoxy]-fluoren-9-one di-p-toluenesulfonate

### Example 2A

### (2R)-1-Boc-2-(methanesulfonyloxymethyl)azetidine

A mixture of (2R)-1-Boc-2-(hydroxymethyl)azetidine (1.5 g, 8.0 mmol; see Tetrahedron Asym. 1998, 9, 2791) and triethylamine (3.5 g, 35 mmol; Spectrum) in dry THF (20 mL) was cooled to 0° C., and methanesulfonyl chloride (0.75 mL, 9.7 mmol; Aldrich) was added slowly with stirring. The reaction mixture was allowed to warm to room temperature and stir for 1 hour, then the solid was removed by filtration and the organic phase was concentrated. The residue was dissolved in dichloromethane (40 mL), washed with water (20 mL), and then concentrated to afford the title compound (800 mg. 3.0 mmol; 38% yield). MS (DCl/NH₃): 266 (M+1)⁺, 283 (M+18)⁺.

### Example 2B

### 2,7-Bis[(2R)-1-Boc-azetidin-2-ylmethoxy]-fluoren-9-one

A mixture of the product of Example 2A (795 mg, 3 mmol), 2,7-dihydroxyfluoren-9-one (212 mg, 1.00 mmol; see Synth. Commun. 1976, 6, 371) and powdered potassium hydroxide (135 mg, 2.4 mmol; Fisher) in DMF (10 mL) was heated to 80° C. with stirring for 16 hours. After cooling to room temperature, the reaction mixture was concentrated. Dichloromethane (10 mL) was added, and the solution was washed with water and brine, concentrated and purified by flash chromatography (80 g silica gel, θ 30% isopropanol-hexanes) to afford the title compound (310 mg, 0.56 mmol; 56% yield). MS (DCl/NH₃): 568 (M+18)⁺.

### Example 2C

### 2,7-Bis[(2R)-azetidin-2-ylmethoxy]-fluoren-9-one di-p-toluenesulfonate

To a solution of the product of Example 2B (310 mg, 0.56 mmol) in ethyl acetate (10 mL) was added p-toluenesulfonic acid monohydrate (213 mg, 1.12 mmol; Aldrich). The mixture was heated to 60° C. with stirring for 16 hours, and the resulting solid was collected by centrifugation to afford the title compound (313 mg, 0.451 mmol; 81% yield). ¹H NMR (300 MHz, methanol-d4): δ 7.70 (4H, d, J=8 Hz), 7.54 (2H, d, J=8 Hz), 7.27 (2H, d, J=2 Hz), 7.22 (4H, d, J=8 Hz), 7.17 (2H, dd, J=8, 2 Hz), 4.86 (2H, m), 4.36 (4H, d, J=4 Hz), 4.18-3.98 (4H, m), 2.73-2.60 (4H, m), 2.35 (6H, s). MS (DCl/NH₃): m/z 351 (M+1)⁺. Anal. Calcd. for C₂₁H₂₂N₂₀₃₂C₇H₈O₃S: C, 60.50; H 5.51; N, 4.03. Found: C, 60.18; H, 5.32; N, 3.91.

### Example 3

### 2,7-Bis[(2R)-1-methylazetidin-2-ylmethoxy]-fluoren-9-one di-p-toluenesulfonate

### Example 3A

### 2,7-Bis[(2R)-1-methylazetidin-2-ylmethoxy]-fluoren-9-one

A mixture of the product of Example 2C (280 mg, 0.403 mmol), formaldehyde (3 mL, 36% aq.; EM Science) and sodium triacetoxyborohydride (400 mg, 1.89 mmol; Aldrich) in water (5 mL) was stirred at room temperature for 16 hours. The mixture was concentrated under vacuum and purified by flash chromatography (80 g silica gel, 1:10:89 NH₄OH:MeOH:CH₂Cl₂) to afford the title compound (150 mg, 0.397 mmol; 98% yield). ¹H NMR (300 MHz, methanol-d4): δ 7.43 (2H, d, J=8 Hz), 7.14 (2H, d, J=3 Hz), 7.05 (2H, dd, J=8, 3 Hz), 4.10-4.02 (4H, m), 3.54 (2H, m), 3.42 (2H, m), 2.97 (2H, dd, J=17, 9 Hz), 2.43 (6H, s), 2.18-2.04 (4H, m). MS (DCl/NH₃): m/z 379 (M+1)⁺.

### Example 3B

### 2,7-Bis[(2R)-1-methylazetidin-2-ylmethoxy]-fluoren-9-one di-p-toluenesulfonate

The product of Example 3A (150 mg, 0.397 mmol) was dissolved in ethyl acetate (5 mL) and ethanol (0.2 mL), and then p-toluenesulfonic acid monohydrate (151 mg, 0.794 mmol; Aldrich) was added. After stirring the mixture for 16 hours, the resulting solid was collected by centrifugation to afford the title compound (202 mg, 0.262 mmol; 66% yield). ¹H NMR (300 MHz, methanol-d4): δ 7.70 (4H, d, J=8 Hz), 7.54 (2H, d, J=8 Hz), 7.27 (2H, d, J=2 Hz), 7.22 (4H, d, J=8 Hz), 7.18 (2H, dd, J=8, 2 Hz), 4.77 (2H, m), 4.47-4.31 (4H, m), 4.24 (2H, m), 4.01 (2H, dd, J=20, 9 Hz), 3.01 (6H, s), 2.69-2.55 (4H, m), 2.35 (6H, s). MS (DCl/NH₃): m/z 379 (M+1)⁺. Anal. Calcd. for C₂₃H₂₆N₂O₃. 2.3C₇H₈O₃S: C, 60.63; H, 5.78; N, 3.62. Found: C, 60.50; H, 5.59; N, 3.47.

### Example 4

### 2,7-Bis[(3S)-pyrrolidin-3-yloxy]-fluoren-9-one di-p-toluenesulfonate

### Example 4A

### 2,7-Bis[(3S)-1-Boc-pyrrolidin-3-yloxy]-fluoren-9-one

A mixture of 2,7-diiodofluoren-9-one (1.2 g, 2.78 mmol; see J. Chem. Res. (S) 1999, 590), (3S)-1-Boc-3-hydroxypyrrolidine (2.0 g, 10.7 mmol; Omega), copper (I) iodide (53 mg, 0.28 mmol; Aldrich), 1,10-phenanthroline (100 mg, 0.56 mol; Aldrich) and powdered cesium carbonate (3.6 g, 11.0 mmol; Aldrich) in toluene (4 mL) was heated to 110° C. with vigorous stirring for 30 h. After cooling to room temperature, the reaction mixture was filtered through diatomaceous earth, rinsing with ethyl acetate and dichloromethane, and the residue purified by flash chromatography (80 g silica gel, 10-80% ethyl acetate-hexanes) to afford the title compound (466 mg, 0.847 mmol, 30% yield). MS (DCl/NH₃): m/z 550 (M)⁺, 568 (M+18)⁺.

### Example 4B

### 2,7-Bis[(3S)-pyrrolidin-3-yloxy]-fluoren-9-one di-p-toluenesulfonate

To a solution of the product of Example 4A (437 mg, 0.795 mmol) in ethyl acetate (10 mL) was added p-toluenesulfonic acid monohydrate (310 mg, 1.63 mmol; Aldrich). The mixture was heated at reflux overnight (16 h), and the resulting orange solid was collected by filtration to afford the title compound (517 mg, 0.744 mmol, 94% yield). ¹H NMR (300 MHz, methanol-d4): δ 7.70 (4H, d, J=8 Hz), 7.52 (2H, d, J=8 Hz), 7.22 (4H, d, J=8 Hz), 7.21 (2H, d, J=2 Hz), 7.12 (2H, dd, J=8, 2 Hz), 5.25 (2H, m), 3.61-3.46 (8H, m), 2.36 (6H, s), 2.37-2.30 (4H, m). MS (DCl/NH₃): m/z 351 (M+1)⁺. Anal. Calcd. for C₂₁H₂₂N₂O₃ 2C₇H₈O₃S: C, 60.50; H, 5.51; N, 4.03. Found: C, 60.64; H, 5.46; N, 3.94.

### Example 5

### 2,7-Bis[(3S)-1-methylpyrrolidin-3-yloxy]-fluoren-9-one di-toluenesulfonate

### Example 5A

### 2,7-Bis[(3S)-1-methylpyrrolidin-3-yloxy]-fluoren-9-one

A solution of the product of Example 4B (400 mg, 0.576 mmol) in aqueous formaldehyde (2 mL, 37%; Fisher) was cooled to 0° C. in an ice bath and treated with excess sodium triacetoxyborohydride (366 mg, 1.73 mmol; Aldrich), added in one portion. The reaction mixture was allowed to warm to ambient temperature overnight, then the pH was adjusted with acid to pH<3 and washed with ether. The aqueous phase was raised to pH>10 with 1 N NaOH and extracted with ether (3x). The organic extract was purified by flash chromatography [80 g silica gel, eluting with 2-20% of 10% NH₄OH/MeOH in CH₂Cl₂] to afford the title compound (230 mg, 100% yield). MS (DCl/NH₃): m/z 379 (M+1)⁺.

### Example 5B

### 2,7-Bis[(S)-1-methylpyrrolidin-3-yloxy]fluoren-9-one di-p-toluenesulfonate

The product of Example 5A (230 mg, 0.576 mmol) was converted to the title compound (325 mg, 0.45 mmol, 78% yield) according to the procedure described in Example 4B. ¹H NMR (300 MHz, methanol-d4): δ 7.70 (4H, d, J=8 Hz), 7.53 (2H, d, J=8 Hz), 7.25-7.17 (6H, m), 7.10 (2H, dd, J=8, 2 Hz), 5.26 (2H, m), 4.12-3.74 (4H, m), 3.47-3.19 (4H, m), 3.02 (6H, br s), 2.76-2.62 (1H, m), 2.45-2.18 (9H, m). MS (DCl/NH₃): m/z 379 (M+1)⁺. Anal. Calcd. for C₂₃H₂₆N₂O₃.2C₇H₈O₃S.0.7H₂O: C, 60.42; H, 5.95; N, 3.81. Found: C, 60.03; H, 5.88; N, 3.82.

### Example 6

### 2,7-Bis-[(3R)-1-azabicyclo[2.2.2]octan-3-yloxy]-fluoren-9-one di-p-toluenesulfonate

### Example 6A

### 2,7-Bis-[(3R)-1-azabicyclo[2.2.2]octan-3-yloxy]-fluoren-9-one

A mixture of 2,7-diiodofluoren-9-one (170 mg, 0.40 mmol; see J. Chem. Research (S), 1999, 590.), (3R)-(−)-quinuclidin-3-ol (318 mg, 2.5 mmol; Acros), copper(I) iodide (38 mg, 0.20 mmol; Aldrich), 1,10-phenanthroline (78 mg, 0.43 mmol; Aldrich) and cesium carbonate (326 mg, 1.0 mmol; Aldrich) in dry toluene (10 mL) was heated to 110° C. and stirred under nitrogen for 60 hours. After cooling to room temperature, the reaction mixture was concentrated and purified by flash chromatography (80 g silica gel, 1:10:89 NH₄OH—MeOH—CH₂Cl₂) to afford the title compound (71 mg, 0.16 mmol; 41% yield). ¹H NMR (300 MHz, methanol-d4) δ 7.43 (2H, d, J=8 Hz), 7.10 (2H, d, J=2 Hz), 7.02 (2H, dd, J=8, 2 Hz), 4.56 (2H, m), 3.01-2.72 (12H, m), 2.17 (2H, m), 2.01 (2H, m), 1.81 (2H, m), 1.70 (2H, m), 1.51 (2H, m). MS (DCl/NH₃): m/z 431 (M+1)⁺.

A mixture of 2-[(3R)-1-azabicyclo[2.2.2]octan-3-yloxy]-7-iodofluoren-9-one and 2-[(3R)-1-azabicyclo[2.2.2]octan-3-yloxy]-fluoren-9-one (140 mg, approximately 1:1) was also collected as byproduct and used without further purification. MS (DCl/NH₃): m/z 306 (M+1)⁺, 432 (M+1)⁺.

### Example 6B

### 2,7-Bis-[(3R)-1-azabicyclo[2.2.2]octan-3-yloxy]-fluoren-9-one di-p-toluenesulfonate

The product of Example 6A (71 mg, 0.16 mmol) was converted to the title compound (118 mg, 0.152 mmol; 92% yield) according to the procedure described in Example 1D. ¹H NMR (300 MHz, methanol-d4): δ 7.70 (4H, d, J=8 Hz), 7.52 (2H, d, J=8 Hz), 7.25-7.18 (6H, m), 7.12 (2H, dd, J=8, 2 Hz), 4.94 (2H, m), 3.82 (2H, dd, J=14, 7 Hz), 3.46-3.32 (10H, m), 2.52 (2H, m), 2.36 (6H, s), 2.30 (2H, m), 2.18-1.83 (6H, m). MS (DCl/NH₃): m/z 431 (M+1)⁺. Anal. Calcd. for C₂₇H₃₀N₂O₃₂.3C₇H₈O₃S: C, 62.63; H, 5.90; N, 3.39. Found: C, 62.56; H, 5.90; N, 3.31.

### Example 7

### 2,7-Bis[(3S)-1-azabicyclo[2.2.2]octan-3-yloxy]-fluoren-9-one fumarate

### Example 7A

### 2,7-Bis[(3S)-1-azabicyclo[2.2.2]octan-3-yloxy]-fluoren-9-one

To a 0° C. mixture of 2,7-dihydroxyfluoren-9-one (113 mg, 0.533 mmol; see Synth. Commun. 1976, 6, 371), (3F)-quinuclidin-3-ol (280 mg, 2.20 mmol; Acros), and polymer-bound triphenylphosphine (933 mg, 3 mmol/g; Aldrich) in THF (5 mL) was added diethylazodicarboxylate (340 μL, 2.16 mmol; Aldrich). After 1 h, the reaction mixture was allowed to warm to room temperature and was stirred over the weekend. The mixture was filtered through diatomaceous earth, the filter pad rinsed with ethyl acetate, and the organic extracts purified by flash chromatography (35 g silica gel, eluting with 2-10% of 10% NH₄OH/MeOH in CH₂Cl₂). Acetonitrile was then added to the residue, and the resulting precipitate collected by filtration to yield the title compound (53 mg, 0.12 mmol, 23% yield). MS (DCl/NH₃): m/z 431 (M+)⁺.

### Example 7B

### 2,7-Bis[(3S)-1-azabicyclo[2.2.2]octan-3-yloxy]-fluoren-9-one fumarate

A solution of the product of Example 7A (53 mg, 0.123 mmol) in ethyl acetate (containing a few drops of ethanol) was treated with a solution of fumaric acid (28 mg, 0.241 mmol; Aldrich) in ethanol. After stirring for 1 h, the precipitate was collected by filtration, affording the title compound (67 mg, 0.11 mmol, 86% yield). ¹H NMR (300 MHz, methanol-d4): δ 7.51 (2H, d, J=8 Hz), 7.18 (2H, d, J=2 Hz), 7.11 (2H, dd, J=8, 2 Hz), 6.68 (1.4H, s), 3.74 (2H, dd, J=15, 8 Hz), 3.36-3.18 (10H, m), 2.46 (2H, m), 2.25 (2H, m), 2.14-1.76 (6H, m). MS (DCl/NH₃): m/z 431 (M+1)⁺. Anal. Calcd. for C₂₇H₃₀N₂O₃.1.4C₄H₄O₄.2.1H₂O: C, 62.07; H, 6.36; N, 4.44. Found: C, 61.83; H, 6.09; N, 4.16.

### Example 8

### 2-[(3R)-1-azabicyclo[2.2.2]octan-3-yloxy]-fluoren-9-one p-toluenesulfonate

### Example 8A

### 2-[(3R)-1-azabicyclo[2.2.2]octan-3-yloxy]-fluoren-9-one

A solution of the byproduct mixture from Example 6A (140 mg) in methanol was treated with 10% palladium on carbon (50 mg) under 1 atm hydrogen (balloon) for 16 hours. The catalyst was filtered off and the resulting solution was concentrated to afford the title compound (112 mg, 0.365 mmol). MS (DCl/NH₃): m/z 306 (M+1)⁺.

### Example 8B

### 2-[(3R)-1-azabicyclo[2.2.2]octan-3-yloxy]-fluoren-9-one p-toluenesulfonate

The product of Example 8A (112 mg, 0.365 mmol) was converted to the title compound (187 mg, 0.36 mmol; 100% yield) according to the procedure described in Example 1D. ¹H NMR (300 MHz, methanol-d4): δ 7.70 (2H, d, J=8 Hz), 7.60-7.48 (4H, m), 7.28 (1H, dd, J=7, 1 Hz), 7.26-7.19 (3H, m), 7.15 (1H, dd, J=8, 2 Hz), 4.96 (1H, m), 3.83 (1H, m), 3.48-3.32 (5H, m), 2.54 (1H, m), 2.36 (3H, s), 2.30 (1H, m), 2.18-1.83 (3H, m). MS (DCl/NH₃): m/z 306 (M+1)⁺. Anal. Calcd. for C₂₀H₁₉NO₂.1.2C₇H₈O₃S: C, 66.62; H, 5.63; N, 2.74. Found: C, 66.65; H, 5.57; N, 2.81.

### Example 9

### 2-[(3S)-1-azabicyclo[2.2.2]-octan-3-yloxy]-fluoren-9-one fumarate

### Example 9A

### 2-[(3S)-1-azabicyclo[2.2.2]-octan-3-yloxy]-fluoren-9-one

To a 0° C. mixture of 2-hydroxyfluoren-9-one (196 mg, 1.00 mmol; Aldrich), (3R)-quinuclidin-3-ol (151 mg, 1.20 mmol; Acros), and polymer-bound triphenylphosphine (500 mg, 3 mmol/g; Aldrich) in THF (5 mL) was added diethylazodicarboxylate (200 μL, 1.27 mmol; Aldrich). After 1 h, the reaction mixture was allowed to warm to room temperature and was stirred overnight (16 h). The mixture was filtered through diatomaceous earth, the filter pad rinsed with dichloromethane, and the organic extracts purified twice by flash chromatography (80 g silica gel, eluting with 1-5% of 10% NH₄OH/MeOH in CH₂Cl₂) to afford the title compound (104 mg, 0.340 mmol, 34% yield). MS (DCl/NH₃): m/z 306 (M+1)⁺.

### Example 9B

### 2-[(3S)-1-azabicyclo[2.2.2]-octan-3-yloxy]-fluoren-9-one fumarate

The product of Example 9A (102 mg, 0.334 mmol) was converted to the title compound (66 mg, 0.16 mmol, 47% yield) according to the procedure described in Example 7B: ¹H NMR (300 MHz, methanol-d4): δ 7.61-7.56 (3H, m), 7.51 (1H, ddd, J=7, 7, 1 Hz), 7.27 (1H, ddd, J=7, 7, 1 Hz), 7.22 (1H, d, J=2 Hz), 7.14 (1H, dd, J=8, 2 Hz), 6.68 (2H, s), 4.93 (1H, m), 3.79 (1H, ddd, J=10, 8, 2 Hz), 3.42-3.23 (5H, m), 2.51 (1H, m), 2.29 (1H, m), 2.161.81 (3H, m). MS (DCl/NH₃): m/z 306 (M+1)⁺. Anal. Calcd. for C₂₀H₁₉NO₂.C₄H₄O₄: C, 68.40; H, 5.50; N, 3.32. Found: C, 68.01; H, 5.47; N, 3.28.

### Example 10

### 2,7-Bis(4-methyl-[1,4]diazepan-1-yl)-fluoren-9-one dihydrochloride

### Example 10A

### 2,7-Bis(4-methyl-[1,4]diazepan-1-yl)-fluoren-9-one

A catalyst solution was prepared by mixing tris(dibenzylideneacetone)dipalladium (Pd₂(dba)₃; 36 mg, 0.039 mmol; Alfa) and racemic 2,2′-bis(diphenylphosphino)-1,1′-binaphthyl (BINAP; 62 mg, 0.10 mmol; Strem) in toluene (1 mL) and heating the mixture to 80° C. for 15 min. The solution was cooled, and then added to a mixture of N-methylhomopiperazine (310 μL, 2.50 mmol; Aldrich) and 2,7-dibromofluoren-9-one (338 mg, 1.00 mmol; Aldrich) in toluene (5 mL). Sodium tert-butoxide (200 mg, 2.08 mmol; Aldrich) was then added, and the reaction mixture was purged with nitrogen and heated to 80-85° C. for 4 h. After cooling to room temperature, the mixture was filtered through diatomaceous earth and purified by chromatography (80 g silica gel, eluting with 2-12% of 10% NH₄OH/MeOH in CH₂Cl₂) to afford the title compound (273 mg, 0.674 mmol, 67% yield): MS (DCl/NH₃): m/z 405 (M+1)⁺.

### Example 10B

### 2,7-Bis(4-methyl-[1,4]diazepan-1-yl)-fluoren-9-one dihydrochloride

To a solution of the product of Example 10A (273 mg, 0.674 mmol) in ethyl acetate containing a few drops ethanol was added a solution of HCl in dioxane (4 M, 335 μL, 1.34 mmol; Aldrich). After stirring the mixture for 2 h, the solid was collected by filtration and recrystallized from hot EtOH/EtOAc to afford the title compound (193 mg, 0.382 mmol, 57% yield). ¹H NMR (300 MHz, D₂O) δ 7.15 (2H, d, J=8 Hz), 6.79-6.75 (4H, m), 3.77 (4H, t, J=4 Hz), 3.52 (4H, t, J=6 Hz), 3.44 (4H, m), 2.97 (6H, s), 2.27 (4H, m). MS (Cl/NH₃): m/z 405 (M+1)⁺. Anal. Calcd. for C₂₅H₃₂N₄O₂HCl.1.5H₂O: C, 59.52; H, 7.39; N, 11.11. Found: C, 59.26; H, 7.44; N, 10.87.

### Example 11

### 2,7-Bis[3,7-diazabicyclo[3.3.0]octan-3-yl]-fluoren-9-one dihydrochloride

### Example 11A

### 2,7-Bis[N-Boc-3,7-diazabicyclo[3.3.0]octan-3-yl]-fluoren-9-one

A catalyst solution was prepared by mixing tris(dibenzylideneacetone)dipalladium (Pd₂(dba)₃; 35 mg, 0.038 mmol; Alfa) and racemic 2,2′-bis(diphenylphosphino)-1,1′ binaphthyl (BINAP; 61 mg, 0.098 mmol; Strem) in toluene (1 mL) and heating the mixture to 80° C. for 15 min. The solution was cooled, and then added to a mixture of 3-Boc-3,7-diazabicyclo[3.3.0]octane (483 mg, 2.28 mmol; see WO 0181347) and 2,7-dibromofluoren-9-one (336 mg, 0.994 mmol; Aldrich) in toluene (5 mL). Sodium tert-butoxide (276 mg, 2.87 mmo; Aldrich) was then added, and the reaction mixture was purged with nitrogen and heated to 80-85° C. overnight (16 h). After cooling to room temperature, the mixture was filtered through diatomaceous earth and purified by chromatography (80 g silica gel, 10-100% EtOAc-hexanes) to afford the title compound (322 mg, 0.537 mmol, 54% yield). MS (DCl/NH₃): m/z 601 (M+1)⁺.

### Example 11B

### 2,7-Bis[3,7-diazabicyclo[3.3.0]octan-3-yl]-fluoren-9-one

A solution of the product of Example 11A (322 mg, 0.537 mmol) in dichloromethane (5 mL) was cooled to 0° C. and treated with trifluoroacetic acid (3 mL). After stirring for 30 min, the reaction mixture was warmed to room temperature and the stirring continued for an additional 30 min. The solution was diluted with dichloromethane, washed with 1 N NaOH (aq), and concentrated to afford the title compound as an oil (225 mg, 0.537 mmol, 100% yield): MS (DCl/NH₃): m/z 401 (M+1)⁺.

### Example 11C

### 2,7-Bis[3,7-diazabicyclo[3.3.0]octan-3-yl]fluoren-9-one dihydrochloride

To a stirred solution of the product of Example 11B (56 mg, 0.14 mmol) in ethyl acetate containing ethanol and methanol was added a solution of HCl in dioxane (4 M; 70 μL, 0.28 mmol; Aldrich). After stirring for 1 h, the purple solid was collected by filtration, affording the title compound (55 mg, 0.12 mmol, 81% yield). ¹H NMR (300 MHz, D₂O): δ 7.24 (2H, d, J=8 Hz), 6.90 (2H, d, J=2 Hz), 6.80 (2H, dd, J=8, 2 Hz), 3.68 (4H, m), 3.47-3.26 (16H, m). MS (DCl/NH₃): m/z 401 (M+1)⁺. Anal. Calcd. for C₂₅H₂₈N₄O.2HCl.0.7H₂O: C, 61.78; H, 6.51; N, 11.53. Found: C, 61.50; H, 6.32; N, 11.21.

### Example 12

### 2,7-Bis[7-methyl-3,7-diazabicyclo[3.3.0]octan-3-yl]fluoren-9-one dihydrochloride

### Example 12A

### 2,7-Bis[7-methyl-3,7-diazabicyclo[3.3.0]octan-3-yl]fluoren-9-one

To a 0° C. suspension of the product of Example 11B (166 mg, 0.415 mmol) in aq. formaldehyde (37%, 3 mL; Fisher) containing a few drops of methanol was added sodium triacetoxyborohydride (298 mg, 1.41 mmol; Aldrich) in one portion. After 30 min, the reaction mixture was allowed to warm to room temperature and stirring was continued overnight (16 h). The mixture was then diluted with dichloromethane, washed with 1 N NaOH, and the aqueous layer was extracted twice with CH₂Cl₂. The combined organic phases were dried over potassium carbonate, filtered, and concentrated. The residue was purified by chromatography (35 g silica gel, eluting with 2-16% of 10% NH₄OH/MeOH in CH₂Cl₂) to afford the title compound (136 mg, 0.318 mmol, 76% yield). MS (DCl/NH₃): m/z 429 (M+1)⁺.

### Example 12B

### 2,7-Bis[7-methyl-3,7-diazabicyclo[3.3.0]octan-3-yl]-fluoren-9-one dihydrochloride

The product of Example 12A (84 mg, 0.20 mmol) was converted to the title compound (85 mg, 0.17 mmol, 85% yield) according to the procedure described in Example 11C. ¹H NMR (300 MHz, methanol-d4): δ 7.32 (2H, d, J=8 Hz), 7.01 (2H, s), 6.85 (2H, d, J=8 Hz), 3.97 (2H, m), 3.67-3.54 (6H, m), 3.453.15 (10H, m), 2.95 (8H, m). MS (DCl/NH₃): m/z 429 (M+1)⁺. Anal. Calcd. for C₂₇H₃₂N₄O.2HCl.0.6H₂O: C, 63.30; H, 6.93; N, 10.94. Found: C, 63.09; H, 7.05; N, 10.87.

### Example 13

### 2-[3,7-diazabicyclo[3.3.0]octan-3-yl]-fluoren-9-one p-toluenesulfonate

### Example 13A

### 2-[3-Boc-3,7-diazabicyclo[3.3.0]octan-3-yl-fluoren-9-one

A mixture of 3-Boc-3,7-diazabicyclo[3.3.0]octane (160 mg, 0.77 mmol; see WO 0181347), 2-bromo-9-fluorenone (200 mg, 0.77 mmol; Aldrich), tris(dibenzylideneacetone)dipalladium (0) (Pd₂ dba₈; 21 mg, 0.023 mmol; Strem), racemic-2,2′-bis(diphenylphosphino)-1,1′-binaphthyl (BINAP; 24 mg, 0.039 mmol; Strem) and Cs₂CO₃ (500 mg, 1.54 mmol; Aldrich) in 20 mL toluene was warmed to 85° C. and stirred for 16 h. The reaction mixture was cooled to ambient temperature, filtered, concentrated under reduced pressure and purified via column chromatography (silica gel, 50% hexanes/EtOAc) to give the title compound (260 mg, 0.67 mmol, 86% yield). MS (DCl/NH₃) m/z 391 (M+H)⁺.

### Example 13B

### 2-[3,7-diazabicyclo[3.3.0]octan-3-yl]-fluoren-9-one

The product of Example 13A (0.26 g, 0.66 mmol) in CH₂Cl₂ (7 mL) was treated with trifluoroacetic acid (5 mL; EM Science) as described in Example 11B to give the title compound (230 mg, 100% yield), which was carried on without purification.

### Example 13C

### 2-[3,7-diazabicyclo[3.3.0]octan-3-yl]-fluoren-9-one p-toluenesulfonate

To the product of Example 13B (50.mg, 0.17 mmol) in 10% CH₃OH/EtOAc (2 mL) was added p-toluenesulfonic acid (33 mg, 0.17 mmol; Aldrich) in 10% CH₃OH/EtOAc (1 mL). A precipitate formed which was isolated via filtration to give the title compound (68 mg, 0.13 mmol, 77% yield). ¹H NMR (methanol-d₄, 300 MHz): δ 7.70 (2H, m), 7.50 (1H, dt, J=7, 1 Hz), 7.43-7.48 (3H, m), 7.22 (2H, m), 7.17 (1H, dt, J=9, 4 Hz), 7.00 (1H, d, J=3 Hz), 6.83 (1H, dd, J=8, 2 Hz), 3.60 (2H, m), 3.52 (2H, m), 3.41 (2H, m), 3.20-3.27 (4H, m), 2.36 (3H, s). MS (DCl/NH₃): m/z 291 (M+H)⁺. Anal. Calcd. for C₁₉H₁₈N₂O.1.25C₇H₈O₃S.0.8H₂O: C, 64.09; H, 5.74; N, 5.39. Found: C, 63.71; H, 5.47; N, 5.80.

### Example 14

### 2-[7-Methyl-3,7-diazabicyclo[3.3.0]octan-3-yl]-fluoren-9-one I-tartrate

### Example 14A

### 2-[7-Methyl-3,7-diazabicyclo[3.3.0]octan-3-yl]-fluoren-9-one

The product of Example 13B (130 mg, 0.46 mmol) was treated with aqueous formaldehyde (5 mL, 37%; EM Science) and NaBH(OAc)₃ (163 mg, 0.77 mmol; Aldrich). After stirring for 3 h, the reaction was quenched with saturated NaHCO₃ (5 mL) and diluted with CH₂Cl₂ (5 mL). The layers were separated and the aqueous phase was extracted with CH₂Cl₂ (3×5 mL). The combined organics were dried over Na₂SO₄, filtered, and concentrated under reduced pressure and purified via column chromatography (silica gel, 1:9:90 NH₄OH/CH₃OH/CH₂Cl₂) to give the title compound (>100%, impure). MS (DCl/NH₃) m/z 305 (M+H)⁺.

### Example 14B

### 2-[7-Methyl-3,7-diazabicyclo[3.3.0]octan-3-yl]-fluoren-9-one I-tartrate

To the product of Example 14A (0.46 mmol) in 10% CH₃OH/EtOAc (2 mL) was added I-tartaric acid (83 mg, 0.55 mmol; Aldrich) in 10% CH₃OH/EtOAc (1 mL). The resulting precipitate was isolated via filtration to afford the title compound (208 mg, 0.45 mmol, 98% yield). ¹H NMR (methanol-d₄, 300 MHz): δ7.49 (1H, dt, J=7, 1 Hz), 7.43-7.47 (3H, m), 7.16 (1H, m), 7.03 (1H, d, J=2 Hz), 6.86 (1H, dd, J=9, 3 Hz), 4.37 (4H, s), 3.52 (2H, m), 3.60 (2H, m), 3.20-3.32 (6H, m), 2.36 (3H, s); MS (DCl/NH₃) m/z 305 (M+H)⁺; Anal. Calcd. for C₂₀H₂₀N₂O.C₄H₆O₆.0.25H₂O: C, 62.80; H, 5.82; N, 6.10; Found: C, 62.44; H, 5.75; N, 5.90.

### Example 15

### 2,7-Bis(3-diethylamino-propyn-1-yl)-fluoren-9-one dihydrochloride

### Example 15A

### 2,7-Bis(3-diethylamino-propyn-1-yl)-fluoren-9-one

A mixture of 2,7-dibromofluoren-9-one (1.00 g, 2.97 mmol; Aldrich), 3-diethylamino-1-propyne (1.6 mL, 11.6 mmol; Lancaster), triethylamine (2 mL, 14.4 mmol; Acros), dichlorobis(triphenylphosphino)palladium (II) (Cl₂Pd(PPh₈)₂; 90 mg, 0.13 mmol; Aldrich), and copper (I) iodide (130 mg, 0.68 mmol; Aldrich) in DMF (30 mL) was heated to 65° C. for 60 h. The reaction mixture was diluted with EtOAc, washed with water and brine, and dried over Na₂SO₄. The residue was purified twice by chromatography (80 g silica gel, eluting with 2-7% of 10% NH₄OH/MeOH in CH₂Cl₂, followed by 80 g silica gel, 1-6% MeOH—CH₂Cl₂) to afford the title compound (1.12 g, 2.81 mmol, 95% yield). MS (DCl/NH₃) m/z 399 (M+H)⁺.

### Example 15B

### 2,7-Bis(3-diethylamino-propyn-1-yl)-fluoren-9-one dihydrochloride

The product from Example 15A (260 mg, 0.65 mmol) was converted to the title compound (280 mg, 0.58 mmol, 89%) according to the procedure described in Example 10B. ¹H NMR (300 MHz, D₂O): δ 7.70 (2H, dd, J=8, 1 Hz), 7.66 (2H, s), 7.58 (2H, d, J=8 Hz), 4.35 (4H, s), 3.43 (8H, q, J=7 Hz), 1.41 (12H, t, J=7 Hz). MS (DCl/NH₃): m/z 399 (M+1)⁺. Anal. Calcd. for C₂₇H₃₀N₂.O₂HCl.0.6H₂O: C, 67.24; H, 6.94; N, 5.81. Found: C, 66.93; H, 7.24; N, 5.82.

### Example 16

### 3,7-Bis(2-diethylaminoethoxy)dibenzothiophene dihydrochloride

### Example 16A

### 3,7-Bis(2-diethylaminoethoxy)dibenzothiophene

To a 0° C. mixture of 3,7-bis(2-diethylaminoethoxy)dibenzothiophene-5,5-dioxide (5.30 g, 1.2 mmol; see J. Med. Chem., 1978, 21, 1084) in dry THF (10 mL) was added portion-wise lithium aluminum hydride (90 mg, 2.4 mmol, Aldrich). The reaction mixture was allowed to warm to ambient temperature and then heated at reflux for 2 hours. After cooling to room temperature, the reaction mixture was quenched by sequential addition of H₂O-THF (1:9, 1 mL), aq. NaOH (2.5 N, 0.1 mL), and H₂O (0.3 mL). The mixture was stirred for 30 minutes, filtered, and the residue was purified by flash chromatography (35 g silica gel, eluting with 4-8% of 10% NF₄OH/MeOH in CH₂Cl₂) to afford the title compound (250 mg, 51% yield).

### Example 16B

### 3,7-Bis(2-diethylaminoethoxy)dibenzothiophene dihydrochloride

To a stirred solution of the product of Example 16A (250 mg, 0.60 mmol) in ethyl acetate (5 mL) and ethanol (0.2 mL), was added a solution of HCl in dioxane (4M; 0.33 mL, 1.32 mmol; Aldrich). After stirring the mixture for 6 hours, the resulting solid was collected by filtration to afford the title compound (284 mg, 0.50 mmol; 93%). ¹H NMR (300 MHz, methanol-d4): δ 8.05 (2H, d, J=9 Hz), 7.54 (2H, d, J=2 Hz), 7.16 (2H, dd, J=9, 2 Hz), 4.46 (4H, t, J=5 Hz), 3.68 (4H, t, J=5 Hz), 3.44-3.35 (8H, q, J=7 Hz), 1.40 (12H, t, J=7 Hz). MS (DCl/NH₃): m/z 415 (M+1)⁺. Anal. Calcd. for C₂₄H₃₄N₂O₂S.2HCl.0.2C₄H₈O₂: C, 58.97; H, 7.50; N, 5.55. Found: C, 58.94; H, 7.43; N, 5.52.

### Example 17

### 3,7-Bis(2-diethylaminoethoxy)dibenzothiophene-5-oxide di-p-toluenesulfonate

### Example 17A

### 3,7-Bis(2-diethylaminoethoxy)dibenzothiophene-5-oxide

To a cooled solution (0° C.) of the product of Example 16A (347 mg, 0.83 mmol) in dry THF (2 mL), was added a solution of CF₃CO₃H in THF (4 M; 0.21 mL, 0.83 mmol). The reaction was stirred 30 minutes at 0° C., then for 30 minutes at room temperature, and concentrated under vacuum. The residue was purified by flash chromatography (35 g silica gel, 1:5:94 NH₄OH—MeOH—CH₂Cl₂) to afford the title compound (360 mg, 100%).

### Example 17B

### 3,7-Bis(2-diethylaminoethoxy)dibenzothiophene-5-oxide di-p-toluenesulfonate

The product of Example 17A (200 mg, 0.46 mmol) was converted to the title compound (218 mg, 0.28 mmol; 61%) according to the procedure of Example 1D. ¹H NMR (300 MHz, methanol-d4): δ 7.89 (2H, d, J=8 Hz), 7.67 (6H, m), 7.33 (4H, dd, J=8, 2 Hz), 7.21 (4H, d, J=8 Hz), 4.45 (4H, t, J=5 Hz), 3.68 (4H, t, J=5 Hz), 3.43-3.32 (8H, m), 2.33 (6H, s), 1.39 (12H, t, J=5 Hz). MS (DCl/NH₃): m/z 431 (M+1)⁺. Anal. Calcd. for C₂₄H₃₄N₂O₃S.2C₇H₈O₃S: C, 58.66; H, 6.50; N, 3.61. Found: C, 58.66; H, 6.53; N, 3.49.

### Example 18

### 3,7-Bis[(3S)-1-azabicyclo[2.2.2]octan-3-yloxy]-dibenzothiophene p-toluenesulfonate

### Example 18A

### 3,7-Bis[(3S)-1-azabicyclo[2.2.2]octan-3-yloxy]-dibenzothiophene

To a 0° C. mixture of 3,7-dihydroxybenzothiophene-5,5-dioxide (300 mg, 1.2 mmol; see J. Med. Chem. 1978, 21, 1084), (3R)-quinuclidin-3-ol (770 mg, 6.0 mmol; Acros), and polymer-bound triphenylphosphine (2.0 g, 3 mmol/g; Aldrich) in THF (12 mL) was added di-tertbutylazodicarboxylate (1.2 mL, 6.0 mmol; Aldrich). The mixture was stirred overnight (16 h) at room temperature, filtered through diatomaceous earth, then rinsed with ethyl acetate. The residue was purified by flash chromatography (35 g silica gel, eluting with 5-10% of 10% NH₄OH/MeOH in CH₂Cl₂) to afford the title compound (150 mg, 27% yield).

### Example 18B

### 3,7-Bis[(3S)-1-azabicyclo[2.2.2]octan-3-yloxy]-dibenzothiophene p-toluenesulfonate

The product of Example 18A (150 mg, 0.32 mmol) was dissolved in ethyl acetate (5 mL) and ethanol (0.2 mL), then p-toluenesulfonic acid monohydrate (122 mg, 0.64 mmol; Aldrich) was added. After stirring the mixture for 16 hours, the resulting solid was collected by filtration to afford the title compound (245 mg, 0.30 mmol; 94%). ¹H NMR (300 MHz, methanol-d4): δ 7.89 (2H, d, J=8 Hz), 7.70 (4H, d, J=8 Hz), 7.47 (2H, d, J=2 Hz), 7.34 (1H, d, J=2 Hz), 7.32 (2H, dd, J=8, 2 Hz), 7.22 (4H, d, J=8 Hz), 5.04 (2H, m), 3.87 (2H, m), 3.5-3.28 (12H, m), 2.55 (2H, m), 2.40-2.21 (7H, m), 2.211.83 (5H, m). MS (DCl/NH₃): m/z 467 (M+1)⁺. Anal. Calcd. for C₂₅H₃₀N₂O₃.2C₇H₈O₃S_{0.5}H₂O: C, 58.59; H, 5.78; N, 3.42. Found: C, 58.58; H, 5.84; N, 3.39.

### Example 19

### 2-[(1S,5S)-3,6-Diazabicyclo[3.2.0]heptan-3-yl]-dibenzothiophene-5,5-dioxide p-toluenesulfonate

### Example 19A

### Benzyl N-(2,2-dimethoxyethyl)carbamate

Benzyl chloroformate (231.3 g, 1.3 mol) was added gradually to a mixture of aminoacetaldehyde dimethyl acetal (152.0 g, 1.3 mol) in toluene (750 mL) and aqueous NaOH (72.8 g, 1.82 mol; in 375 mL of water) at 10-20° C. After the addition was complete, the mixture was stirred at ambient temperature for 4 h. The organic layer was separated, washed with brine (2×100 mL) and concentrated to provide the title compound. MS (DCl/NH₃): m/z 240 (M+1)⁺, 257 (M+18)⁺.

### Example 19B

### Benzyl N-allyl-N-(2,2-dimethoxyethyl)carbamate

The product of Example 19A (281.0 g, 1.18 mol) in dry toluene (1.0 L) was treated with powdered potassium hydroxide (291.2 g, 5.20 mol) and triethylbenzylammonium chloride (4.4 g, 0.02 mol). A solution of allyl bromide (188.7 g, 1.56 mol) in toluene (300 mL) was then added dropwise over 1 h at 2030° C. The mixture was stirred overnight at room temperature and then water (300 mL) was added over 20 min at 20-30° C. The layers were separated and the aqueous phase was extracted with toluene (2×300 mL). The organic phases were combined, washed with brine (2×100 mL), dried (K₂CO₃), filtered and the filtrate concentrated to provide the title compound. MS (DCl/NH₃): m/z 280 (M+1)⁺, 297 (M+18)⁺.

### Example 19C

### Benzyl N-allyl-N-(2-oxoethyl)carbamate

The product of Example 19B (314.0 g, 1.125 mol) was treated with formic acid (88%, 350 mL) at room temperature and allowed to stir for 15 h. Most of the formic acid was removed by concentration under reduced pressure at 40-50° C. The residue was extracted with ethyl acetate (3×500 mL). The extracts were combined and washed with brine until the wash had a pH=6-7. The organic phase was concentrated to provide the title compound. MS (DCl/NH₃): m/z 234 (M+1)⁺.

### Example 19D

### Benzyl N-allyl-N-[2-(hydroxyimino)ethyl]carbamate

The product of Example 19C (260 g, 1.115 mol) in acetonitrile (1.5 L) was treated with sodium acetate trihydrate (170.6 g, 4.41 mol) in distilled water (750 mL) and hydroxylamine hydrochloride (98.0 g, 4.41 mol) under nitrogen. The mixture was stirred at room temperature for about 20 h. The volatiles were removed under reduced pressure and the residue was extracted with ethyl acetate (2×750 mL). The combined organic phases were washed with brine until the wash had a pH=7. The organic phase was concentrated to provide the title compound. MS (DCl/NH₃): m/z 249 (M+1)⁺, 266 (M+18)⁺.

### Example 19E

### Benzyl cis-3-amino-4-(hydroxymethyl)-1-pyrrolidine carboxylate

A solution of the product of Example 19D (240 g, 0.97 mol) in xylenes (1.0 L) was heated at reflux under nitrogen for about 10 h. The resulting brown solution was cooled to 10-15° C. and acetic acid (1.0 L) was added under N₂. Zinc powder (100 g, 1.54 mol) was added gradually, and the gray mixture was stirred at room temperature for 3 h. The mixture was filtered and water (1.0 L) was added to the filtrate. The filtrate was stirred for 10 min and the organic layer was separated. The aqueous phase was washed with xylenes (4×400 mL) and then concentrated under reduced pressure to a volume of approximately 200 mL. The pH of the residue was adjusted with base to pH 910 by addition of saturated aqueous Na₂CO₃. The precipitated white solid was removed by filtration and the filtrate was extracted with chloroform (3×600 mL). The combined organic phases were washed with saturated Na₂CO₃ solution (2×50 mL) and dried over anhydrous Na₂CO₃. The mixture was filtered through a short column of diatomaceous earth and the filtrate was concentrated to provide the title compound. MS (DCl/NH): m/z 251 (M+1)⁺.

### Example 19F

### Benzyl (4aS,7aS)-2,2-dimethylhexahydropyrrolo[3,4d][1,3]oxazine-6(4H)-carboxylate (R)-mandelate

The product of Example 19E (140 g, 0.56 mol) in dry acetone (150 mL) was treated with 2-methoxypropene (55 mL, 0.57 mol) at room temperature overnight. The reaction mixture was concentrated under reduced pressure and the residue was dissolved in dry acetone (750 mL). R)-Mandelic acid (85 g, 0.56 mol) was added and the solution was stirred at room temperature for 48 h. The precipitate was isolated by filtration and dried under reduced pressure to provide the title compound as a solid. MS (DCl/NH₃): m/z 291 (M+1)⁺.

### Example 19G

### Benzyl (3S,4S)-3-[(tert-butoxycarbonyl)amino]-4-(hydroxymethyl)-1-pyrrolidinecarboxylate

The product of Example 19F (56 g, 127 mmol) in ethanol (50 mL) was treated with 5% aqueous sulfuric acid (100 mL) at room temperature and allowed to stir for 16 h. The pH of the mixture was adjusted with base to pH˜10 with 20% aqueous sodium hydroxide (50 mL) and then the mixture was treated with di-tertbutyl dicarbonate (41.5 g, 190 mmol) in ethanol (50 mL) at 10-20° C. After stirring at room temperature for 4 h, the ethanol was removed under reduced pressure and the residue was extracted with ethyl acetate (3×500 mL). The combined organic phases were washed with brine (2×100 mL) and concentrated to provide the title compound. MS (DCl/NH₃): m/z 351 (M+1)⁺, 368 (M+18)⁺. The enantiomeric purity of the title compound was determined to be ≧99% ee by chiral HPLC (Chiracel AD column; ethanol/hexanes=20/80, 1.0 mL/minute, UV 220 nm; retention time 10.8 min).

### Example 19H

### Benzyl (3S,4S)-3-[(tert-butoxycarbonyl)amino]-4-{(methylsulfonyl)oxy]methyl}-1-pyrrolidinecarboxylate

The product of Example 19G (43.7 g, 125 mmol) and triethylamine (25.2 g, 250 mmol) in CH₂Cl₂ (600 mL) were treated with methanesulfonyl chloride (12.6 mL, 163 mmol) over 30 minutes at −10° C. The solution was allowed to warm to room temperature over 1 h and quenched with water (100 mL). The layers were separated and the aqueous phase was extracted with CH₂Cl₂ (2×400 mL). The combined organic phases were washed with brine (2×100 mL), dried over Na₂SO₄, filtered, and the filtrate concentrated to provide the title compound. MS (DCl/NH₃): m/z 429 (M+1)⁺, 446 (M+18)⁺.

### Example 19I

### Benzyl (3S,4S)-3-amino-4-{[(methylsulfonyl)oxy]methyl}1-pyrrolidinecarboxylate trifluoroacetate

The product of Example 19H (43.7 g, 125 mmol) in CH₂Cl₂ (150 mL) was treated with trifluoroacetic acid (50 mL) at room temperature and allowed to stir for 1 h. The mixture was concentrated under reduced pressure to give the title compound. MS (DCl/NH₃): m/z 329 (M+1)⁺.

### Example 19J

### Benzyl (1S,5S)-3,6-diazabicyclo[3.2.0]heptane-3-carboxylate

The product of Example 191 was dissolved in ethanol (250 mL) and the pH was adjusted with base to pH ˜12 with 25% aqueous NaOH. The mixture was warmed to 60° C. for 1.5 h, then allowed to cool to room temperature and used in the next step without further purification. An analytical sample was removed (−1 mL) and concentrated under reduced pressure. The residue was extracted with chloroform (2×5 mL). The extracts were combined, washed with brine (3×2 mL) and then passed through a short column of diatomaceous earth. The filtrate was concentrated to provide an analytical amount of the title compound. MS (DCl/NH₃): m/z 233 (M+H)⁺, 250 (M+NH₄)⁺.

### Example 19K

### 6-Boc-3-carboxybenzyl-(1R,5S)-3,6-diazabicyclo[3.2.0]heptane

The solution of Example 19J was slowly added to di-tert-butyl dicarbonate (40.9 g, 188 mmol) in ethanol (50 mL) over 30 min a room temperature. The mixture was stirred at room temperature for additional 0.5-1 h, then concentrated under reduced pressure. The residue was extracted with ethyl acetate (3×500 mL). The ethyl acetate extracts were combined, washed with brine (3×50 mL), stirred with KHSO₄ (5%, 100 mL) for 10 min and the phases separated. The organic layer was washed with brine (3×50 mL) and passed through a short column of diatomaceous earth. The filtrate was concentrated to provide the title compound which was used in the next step without further purification. MS (DCl/NH₃): m/z 333 (M+1)⁺.

### Example 19L

### tert-Butyl (1R,5S)-3,6-diazabicyclo[3.2.0]heptane-6-carboxylate

The product of Example 19K (40.0 g, 0.120 mol) was dissolved in methanol (400 mL) and treated with Pd/C (10 wt %, 4.0 g) under hydrogen at room temperature for 10 h. The reaction mixture was filtered through a short column of diatomaceous earth and the filtrate was concentrated to provide the title compound. MS (DCl/NH₃): m/z 199 (M+1)⁺.

### Example 19M

### 2,8-Dibromo-dibenzothiophene 5,5-dioxide

To a solution of 2,8-dibromodibenzothiophene (2.50 g, 7.4 mmol; TCl-US) in acetic acid (20 mL; EM Science) was added 30% hydrogen peroxide (10 mL; JT Baker). The solution was heated at reflux overnight (16 h), then cooled to room temperature, 25 mL of water added and the resulting solid was collected by filtration, washed with excess water to afford the title compound (1.65 g, 4.4 mmol, 60% yield). MS(DCl/NH₃): m/z 392 (M+18)⁺.

### Example 19N

### 2-Bromo-8-[6-Boc-(1R,5S)-3,6-diazabicyclo[3.2.0]heptanyl]-dibenzothiophene-5,5-dioxide

A catalyst solution was prepared by mixing tris(dibenzylideneacetone)dipalladium (Pd₂(dba)₃; 40 mg, 0.043 mmol; Alfa) and racemic 2,2-′bis(diphenylphosphino)-1,1′-binaphthyl (BINAP; 54 mg, 0.086 mmol; Strem) in toluene (10 mL) and heating the mixture to 80° C. for 15 min. The solution was cooled and then was added the products of Example 19M (800 mg, 2.15 mmol) and Example 19L (640 mg, 3.2 mmol;) in toluene (5 mL). Cesium carbonate (1.05 g, 3.2 mmol; Aldrich) was then added, and the reaction mixture was purged with nitrogen and heated to 80-85° C. for 16 h. After cooling to room temperature, the mixture was filtered through diatomaceous earth and purified by chromatography (80 g silica gel, 50:48:2 ethyl acetate-hexane diethylamine) to afford the title compound (100 mg, 0.20 mmol, 10% yield): MS (DCl/NH₃): m/z 492 (M+1)⁺.

### Example 19O

### 2-[6-Boc-(1R,5S)-3,6-diazabicyclo[3.2.0]heptan-3-yl]-dibenzothiophene-5,5-dioxide

A solution of the product from Example 19N (100 mg) in ethanol was treated with 10% palladium on carbon (50 mg) under 1 atm hydrogen (balloon) for 16 hours. The catalyst was filtered off and the resulting solution was concentrated to afford the title compound (55 mg, 0.13 mmol). MS (DCl/NH₃): m/z 413 (M+1)⁺.

### Example 19P

### 2-[(1S,5S)-3,6-diazabicyclo[3.2.0]heptan-3-yl]-dibenzothiophene-5,5-dioxide

A solution of the product of Example 190 (55 mg, 0.13 mmol) in dichloromethane (5 mL) was cooled to 0° C. and treated with trifluoroacetic acid (3 mL). After stirring for 30 min, the reaction mixture was warmed to room temperature and the stirring continued for an additional 30 min. The solution was diluted with dichloromethane, washed with 1 N NaOH (aq), concentrated, and purified by flash chromatography (20 g silica gel, 1:10:89 NH₄OH:MeOH:CH₂Cl₂) to afford the title compound (37 mg, 0.12 mmol, 89% yield). MS (DCl/NH₃): m/z 313 (M+H)⁺.

### Example 19Q

### 2-[(1S,5S)-3,6-diazabicyclo[3.2.0]heptan-3-yl]-dibenzothiophene-5,5-dioxide p-toluenesulfonate

The product of Example 19P (37 mg, 0.12 mmol) was dissolved in ethyl acetate (5 mL) and ethanol (0.2 mL), then p-toluenesulfonic acid monohydrate (27 mg, 0.14 mmol; Aldrich) was added. After stirring the mixture for 16 hours, the resulting solid was collected by filtration to afford the title compound (36.4 mg, 0.09 mmol; 64%): ¹H NMR (300 MHz, methanol-d4) δ 8.04 (1H, d, J=8 Hz), 7.78-7.58 (6H, m), 7.43 (1H, d, J=2 Hz), 7.20 (2H, d, J=8 Hz), 7.04 (1H, dd, J=8, 2 Hz), 5.10 (1H, t, J=2 Hz), 4.30 (2H, m)4.08 (1H, d, J=8 Hz), 3.75 (1H, m), 3.59 (1H, m) 3.35-3.19 (2H, m), 2.34 (3H, s). MS (DCl/NH₃): m/z 313 (M+1)⁺. Anal. Calcd. for C₁₇H₁₆N₂O₂S.C₇H₈O₃S: C, 59.48; H, 4.99; N, 5.78. Found: C, 59.19; H, 4.78; N, 5.65.

### Example 20

### 2-Amino-7-[3,7-diazabicyclo[3.3.0]octan-3-yl]-fluoren-9-one dihydrochloride

### Example 20A

### 2-[N-Boc-3,7-diazabicyclo[3.3.0]octan-3-yl]-7-bromofluoren-9-one

A mixture of 3-Boc-3,7-diazabicyclo[3.3.0]octane (2.12 g, 10.0 mmol; see WO 0181347), 2,7-dibromofluoren-9-one (6.76 g, 20.0 mmol; Aldrich), tris(dibenzylideneacetone)dipalladium (0) (Pd₂ dba₈; 185 mg, 0.202 mmol; Strem), racemic-2,2′-bis(diphenylphosphino)-1,1′-binaphthyl (BINAP; 310 mg, 0.498 mmol; Strem) and sodium tert-butoxide (1.4 g, 14.6 mmol; Aldrich) in 100 mL toluene was warmed to 85° C. and stirred for 9 h. The reaction mixture was cooled to ambient temperature and filtered through diatomaceous earth, rinsing with dichloromethane. After concentrating the solution under reduced pressure, the residue was purified by column chromatography (silica gel, 10-60% EtOAc/hexanes) to give the title compound (3.54 g, 7.55 mmol, 75% yield). MS (DCl/NH₃): m/z 469, 471 (M+1)⁺.

### Example 20B

### 2-[N-Boc-3,7-diazabicyclo[3.3.0]octan-3-yl]-7-(diphenylmethyleneamino)-fluoren-9-one

The product of Example 20A (494 mg, 1.05 mmol), benzophenone imine (220 μL, 1.31 mmol; Aldrich), tris(dibenzylideneacetone)dipalladium (0) (Pd₂dba₃; 19 mg, 0.021 mmol; Strem), racemic-2,2′-bis(diphenylphosphino)-1,1′-binaphthyl (BINAP; 39 mg, 0.063 mmol; Strem) and sodium tertbutoxide (140 mg, 1.46 mmol; Aldrich) in 5 mL toluene were warmed to 80° C. and stirred for 18 h. The reaction mixture was cooled to ambient temperature and filtered through diatomaceous earth, rinsing with dichloromethane. After concentrating the solution under reduced pressure, the residue was purified by column chromatography (silica gel, 10-60% EtOAc/hexanes) to give the title compound (572 mg, 0.958 mmol, 96% yield). MS (DCl/NHi: m/z 570 (M+1)⁺.

### Example 20C

### 2-Amino-7-[N-Boc-3,7-diazabicyclo[3.3.0]octan-3-yl]-fluoren-9-one

To a solution of the product of Example 20B (572 mg, 0.958 mmol) in THF (5 mL) was added 5 drops aqueous 2 N HCl. The reaction mixture was stirred for 4 h, then diluted with dichloromethane, washed with aqueous 1 N NaOH, and concentrated under reduced pressure. The residue was purified by column chromatography (silica gel, 10-100% EtOAc/hexanes) to afford the title compound (353 mg, 0.872 mmol, 91% yield). MS (DCl/NH₃): m/z 406 (M+1)⁺.

### Example 20D

### 2-Amino-7-[3,7-diazabicyclo[3.3.0]Octan-3-yl]-fluoren-9-one dihydrochloride

A solution of the product of Example 20C (126 mg, 0.311 mmol) in dichloromethane (2 mL) was cooled to 0° C. and treated with trifluoroacetic acid (2 mL). After stirring for 30 min, the reaction mixture was warmed to room temperature and the stirring continued for an additional 1 h. The solution was diluted with dichloromethane, washed with dilute Na₂CO₃ (aq), and concentrated to afford the free base of the title compound (97 mg, 0.32 mmol, 100% yield). This material was dissolved in ethanol containing a few drops of methanol and treated with a solution of HCl in dioxane (4 M, 150 μL, 0.60 mmol; Aldrich). After stirring for 1 h, the solution was concentrated and the residue triturated with EtOH-EtOAc to afford the title compound: ¹H NMR (300 MHz, methanol-d4) δ 7.62 (1H, dd, J=7, 1 Hz), 7.55 (1H, d, J=8 Hz), 7.46 (2H, m), 7.04 (1H, d, J=2 Hz), 6.88 (1H, dd, J=8, 2 Hz), 3.68-3.43 (6H, m), 3.303.21 (4H, m). MS (DCl/NH₃): m/z 306 (M+1)⁺. Anal. Calcd. for C₁₉H₁₉N₃O.1.9HCl.0.1C₄H₈O₂: C, 60.77; H, 5.70; N, 10.96. Found: C, 61.09; H, 5.41; N, 10.66.

### Example 21

### 2-[(3R)-1-Azabicyclo[2.2.2]octan-3-yloxy]-xanthen-9-one trifluoroacetate

### Example 21A

### (3R)-1-azabicyclo[2.2.2]octan-3-ol

(R)-3-Quinuclidinol hydrochloride (20 g, 12.2 mmol; Aldrich) was treated with aq. NaOH (20%, 50 mL) at ambient temperature for 10 min, then extracted with CHCl₃/iPrOH (10:1, 3×200 mL). The extracts were combined, washed with brine (50 mL) and dried over MgSO₄. After removal of the drying agents by filtration, the filtrate was concentrated under reduced pressure to afford the title compound as white solid (15.5 g, 122 mmol, 99% yield). ¹H NMR (300 MHz, methanol-d4) δ 3.88-3.82 (1H, m), 3.10 (1H, ddd, J=14, 8, 2 Hz), 2.95-2.50 (5H, m), 2.05-1.90 (1H, m), 1.851.76 (2H, m), 1.60-1.52 (1H, m), 1.50-1.36 (1H, m). MS (DCl/NH₃): m/z 128 (M+1)⁺.

### Example 21B

### 2-[(3R)-1-Azabicyclo[2.2.2]octan-3-yloxy]-xanthen-9-one

The product from Example 21A (256 mg, 2.02 mmol) was combined with 2-iodoxanthen-9-one (322 mg, 1.00 mmol; see J. Chem. Research (S) 1999, 590.), copper(I) iodide (20 mg, 0.11 mmol; Aldrich), 1,10-phenanthroline (36 mg, 0.20 mmol; Aldrich) and powdered cesium carbonate (500 mg, 1.53 mmol; Aldrich) in dry toluene (1 mL) was heated to 110° C. and stirred under nitrogen for 36 hours. After cooling to room temperature, the reaction mixture was filtered through diatomaceous earth, rinsing with dichloromethane, concentrated and purified by flash chromatography (80 g silica gel, 1:10:89 NH₄OH:MeOH:CH₂Cl₂). The resulting material (250 mg) was repurified by reverse-phase HPLC (40×100 mm Symmetry-C₈, 5-30% aq. TFA (0.1%)-MeCN) to afford the title compound (57 mg, 0.18 mmol, 18% yield). MS (DCl/NH₃): m/z 322 (M+1)⁺.

### Example 21C

### 2-[(3R)-1-Azabicyclo[2.2.2]octan-3-yloxy]-xanthen-9-one trifluoroacetate

The product from Example 21B (57 mg, 0.18 mmol) was dissolved in methanol (500 μL) and treated with trifluoroacetic acid (2 drops). The mixture was diluted with ether (5 mL) and stirred at room temperature for 1 h. The resulting precipitate was collected by centrifugation and washed with ether, affording the title compound (44 mg, 0.10 mmol, 56% yield): ¹H NMR (300 MHz, methanol-d4) δ 8.29 (1H, dd, J=8, 2 Hz), 7.85 (1H, ddd, J=8, 7, 2 Hz), 7.72 (1H, d, J=7 Hz), 7.65 (1H, d, J=9 Hz), 7.62 (1H, d, J=8 Hz), 7.54 (1H, dd, J=9, 3 Hz), 7.47 (1H, ddd, J=8, 7, 1 Hz), 5.03 (1H, m), 3.89 (1H, ddd, J=8, 7, 2 Hz), 3.48-3.25 (5H, m), 2.59 (1H, m), 2.34 (1H, m), 2.21-2.00 (2H, m), 1.91 (1H, m). MS (DCl/NH₃): m/z 322 (M+1)⁺. Anal. Calcd. for C₂₀H₁₉NO₃.C₂HF₃O₂: C, 60.69; H, 4.63; N, 3.22. Found: C, 60.36; H, 4.28; N, 3.10.

### Example 22

### 2-(1-Azabicyclo[2.2.2]octan-3-yloxy)-9H-carbazole

To a 0° C. mixture of 2-hydroxy-9H-carbazole (369 mg, 2.02 mmol; Aldrich), quinuclidin-3-ol (260 mg, 2.05 mmol; Aldrich), and triphenylphosphine (646 mg, 2.47 mmol; Aldrich) in THF (10 mL) was added diethylazodicarboxylate (320 μL, 2.03 mmol; Lancaster). After 1 h, the reaction mixture was allowed to warm to room temperature and was stirred for 3 d. The mixture was diluted with dichloromethane, washed with saturated aq. NaHCO₃, dried over MgSO₄, and purified by flash chromatography (80 g silica gel, eluting with 1-16% of 10% NH₄OH/MeOH in CH₂Cl₂) to afford the title compound as an oil (350 mg, 1.20 mmol, 59% yield). Trituration d the oil with ethyl acetate produced a solid. ¹H NMR (300 MHz, methanol-d4) δ 7.92 (1H, d, J=6 Hz), 7.90 (1H, d, J=8 Hz), 7.37 (1H, d, J=8 Hz), 7.26 (1H, ddd, J=7, 7, 1 Hz), 7.10 (1H, ddd, J=8, 8, 1 Hz), 6.92 (1H, d, J=2 Hz), 6.78 (1H, dd, J=8, 2 Hz), 4.59 (1H, m), 3.36 (1H, ddd, J=14, 8, 2 Hz), 3.042.75 (5H, m), 2.22 (1H, m), 2.11 (1H, m), 1.88-1.65 (2H, m), 1.51(1H, m). MS (DCl/NH₃): m/z 293 (M+1)⁺. Anal. Calcd. for C₁₉H₂₀N₂O 0.1C₄H₈O₂: C, 77.36; H, 6.96; N, 9.30. Found: C, 77.04; H, 7.23; N, 9.45.

### Example 23

### Determination of Biological Activity

To determine the effectiveness of representative compounds of this invention as α7 nAChRs, the compounds of the invention were evaluated according to the [³H]-methyllycaconitine (MLA) binding assay and considering the [³H]-cytisine binding assay, which were performed as described below.

[³H-Cytisine Binding

Binding conditions were modified from the procedures described in Pabreza L A, Dhawan, S, Kellar K J, [³H]-Cytisine Binding to Nicotinic Cholinergic Receptors in Brain, Mol. Pharm. 39: 9-12, 1991. Membrane enriched fractions from rat brain minus cerebellum (ABS Inc., Wilmington, Del.) were slowly thawed at 40° C., washed and resuspended in 30 volumes of BSS-Tris buffer (120 mM NaCl/5 mM KCl/2 mM CaCl₂/2 mM MgCl₂/50 mM Tris-Cl, pH 7.4, 4° C.). Samples containing 100-200 μg of protein and 0.75 nM [³H]-cytisine (30 Cᵢ/mmol; Perkin Elmer/NEN Life Science Products, Boston, Mass.) were incubated in a final volume of 500 μL for 75 minutes at 4° C. Seven log-dilution concentrations of each compound were tested in duplicate. Non-specific binding was determined in the presence of 10 μM (-)-nicotine. Bound radioactivity was isolated by vacuum filtration onto prewetted glass fiber filter plates (Millipore, Bedford, Mass.) using a 96-well filtration apparatus (Packard Instruments, Meriden, Conn.) and were then rapidly rinsed with 2 mL of ice-cold BSS buffer (120 mM NaCl/5 mM KCl/2 mM CaCl₂/2 mM MgCl₂). Packard MicroScint-20® scintillation cocktail (40 μL) was added to each well and radioactivity determined using a Packard TopCount® instrument. The IC₅₀ values were determined by nonlinear regression in Microsoft Excel® software. Kᵢ values were calculated from the IC₅₀s using the Cheng-Prusoff equation, where Kᵢ=IC₅₀/1+[Ligand]/K_{D}].

[³H]-Methyllycaconitine (MLA) Binding

Binding conditions were similar to those for [³H]-cytisine binding. Membrane enriched fractions from rat brain minus cerebellum (ABS Inc., Wilmington, Del.) were slowly thawed at 4° C., washed and resuspended in 30 volumes of BSS-Tris buffer (120 mM NaCl, 5 mM KCl, 2 mM CaCl₂,2 mM MgCl₂, and 50 mM Tris-Cl, pH 7.4, 22° C.). Samples containing 100-200 μg of protein, 5 nM [3H]-MLA (25 Cᵢ/mmol; Perkin Elmer/NEN Life Science Products, Boston, Mass.) and 0.1% bovine serum albumin (BSA, Millipore, Bedford, Mass.) were incubated in a final volume of 500 μL for 60 minutes at 22° C. Seven log-dilution concentrations of each compound were tested in duplicate. Non-specific binding was determined in the presence of 10 μM MLA. Bound radioactivity was isolated by vacuum filtration onto glass fiber filter plates prewetted with 2% BSA using a 96-well filtration apparatus (Packard Instruments, Meriden, Conn.) and were then rapidly rinsed with 2 mL of ice-cold BSS. Packard MicroScint-20® scintillation cocktail (40 μL) was added to each well and radioactivity was determined using a Packard TopCount® instrument. The IC₅₀ values were determined by nonlinear regression in Microsoft Excel® software. Kᵢ values were calculated from the IC₅₀s using the Cheng-Prusoff equation, where Kᵢ=IC₅₀/1+[Ligand]/K_{D}].

Compounds of the invention had Kᵢ values of from about 1 nanomolar to about 10 micromolar when tested by the [³H]-MLA assay, many having a Kᵢ of less than 1 micromolar. [³H]-Cytisine binding values of compounds of the invention ranged from about 50 nanomolar to at least 100 micromolar. The determination of preferred compounds typically considered the Kᵢ value as measured by MLA assay in view of the Kᵢ value as measured by [³H]-cytisine binding, such that in the formula D=Kᵢ³_{H-cytisine}/K_{i MLA}, D is about 50. Preferred compounds typically exhibited greater potency at α7 receptors compared to α4β2 receptors.

Compounds of the invention are α7 nAChRs ligands that modulate function of α7 nAChRs by altering the activity of the receptor. The compounds can be inverse agonists that inhibit the basal activity of the receptor or antagonists that completely block the action of receptor-activating agonists. The compounds also can be partial agonists that partially block or partially activate the α7 nAChRreceptor or agonists that activate the receptor.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof.

1. A compound of the formula (I): 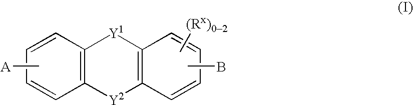
or a pharmaceutically acceptable salt, ester, amide, or prodrug hereof, wherein:
A and B are each independently selected from the group consisting of hydrogen; halogen; alkoxy; amino; alkylamino; acylamino; dialkylamino; cyano; nitro; and —SO₃H; and
a group of formula (a): 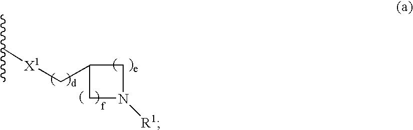
a group of formula (b): 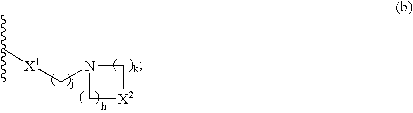
group of formula (c): 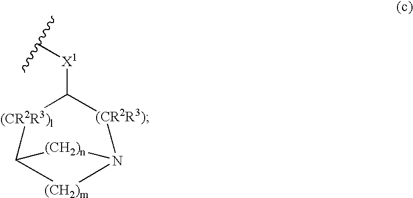
a group of formula (d): 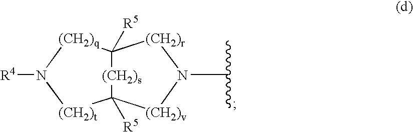
a group of formula (e): 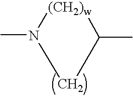
a group of formula (f): 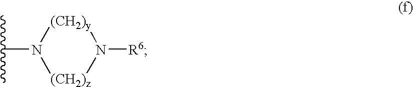
a group of formula (g): 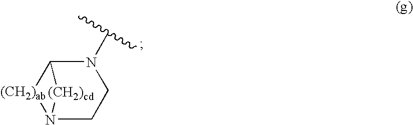
(h) —C≡CCH₂NR⁷R⁸; and (i) —O—(C(R²⁰)₂₋₃N(R²¹)(R²²); provided that at least one of A or B is a group selected from (a)-(i); with the proviso that if A or B is selected from group (a), (b), or (f) when y and z are both two, then A and B are different;
X¹ at each occurrence is selected from the group consisting of O, S, and —N(R⁹)—;
X² at each occurrence is selected from the group consisting of O, S, —CH₂—, and —N(R¹⁰)—;
Y¹ is independently selected from the group consisting of —C(O)—, —CH₂—, —CH(OH)—, —C(S)—, —N(R¹¹)—, —O—, —S—, —S(O)—, —S(O)₂—, —C(O)NH—, and —S(O)₂NH—, provided that if Y¹ is —C(O)—, —O—, —S—, or —N(R¹¹ and one of A or B is selected from a group (a), (b), or (f), then the other of A or B is selected from the group consisting of dialkylamino, cyano, and —SO₃H;
Y² is a bond or Y² is independently selected from −O—, —S—, and —N(R¹²)—;
R¹ is independently selected from hydrogen and alkyl;
R² and R³ at each occurrence are each independently selected from the group consisting of hydrogen and alkyl;
R⁴ and R⁶ at each occurrence are each independently selected from the group consisting of hydrogen and alkyl;
R⁵ at each occurrence are each independently selected from the group consisting of hydrogen, alkyl, and alkoxycarbonyl;
R⁷ and R⁸ are each independently selected from hydrogen and alkyl or R⁷ and R⁸ taken together with the nitrogen atom to which each is attached form a 4- to 8-membered cyclic amine;
R⁹, R¹⁰, R¹¹, and R¹² at each occurrence are each independently selected from hydrogen and alkyl;
R¹⁶ and R¹⁷ are each independently selected from hydrogen and alkyl, or R¹⁶ and R¹⁷ taken together with the nitrogen atom to which each is attached form a 4 to 8-membered cyclic amine;
R²⁰ is selected from the group consisting of hydrogen and alkyl;
R²¹ and R²² are each independently selected from the group consisting of hydrogen and alkyl;
R^{x} is independently selected at each occurrence from the group consisting of hydrogen, halogen, alkoxy, amino, alkylamino, dialkylamino, acylamino, dialkylaminoalkyl, and cyano;
d is independently selected from 0 or 1;
e and f are each independently selected from 0, 1, 2 or 3 provided that the sum total of e and f is 2, 3, or 4, provided that when d is 0, e and f are selected from 1, 2 or 3;
j is independently selected from 2 or 3;
h and k are each independently selected from 0, 1, or 2, provided that the sum total of h and k is 2, 3, or 4, provided that when X² is O, S, or N(R¹⁰), h and k are both 2;
l is 0 or 1, m is 2 or 3, and n is 0, 1, or 2, provided that the sum total of/, m, and n is 4, 5, or 6;
q, r, s, t, and v are each independently selected from 0, 1, or 2, provided that the sum of q and r; t and v; q, s, and t; and r, s, and v; are each at least 1, and further provided that the sum total of q, r, s, t, and v is 2, 3, 4, or 5, provided that when the sum total is 5 and Y¹ is —O—, —S—, or —N(R¹¹)— and Y² is a bond, both A and B are other than hydrogen;
w and x are each independently selected from 1, 2, or 3, provided that the sum total of w and x is 3, 4, 5, or 6;
y and z are each independently selected from 2, 3, or 4, provided that the sum total of y and z is 4, 5, or 6; and
ab is 2 or 3, and cd is 1 or 2.

2. The compound according to claim 1, wherein the group of formula (a) is selected from the group consisting of azetidinyloxy, N-methylazetidinyloxy, pyrrolidinyloxy, N-methylpyrrolidinyloxy, piperidinyloxy, N-methylpiperidinyloxy; azetidinylmethoxy, N-methylazetidinylmethoxy, pyrrolidinylmethoxy, N-methylpyrrolidinylmethoxy, piperidinylmethoxy, and N-methylpiperidinylmethoxy.

3. The compound according to claim 1, wherein the group of formula (b) is selected from the group consisting of:

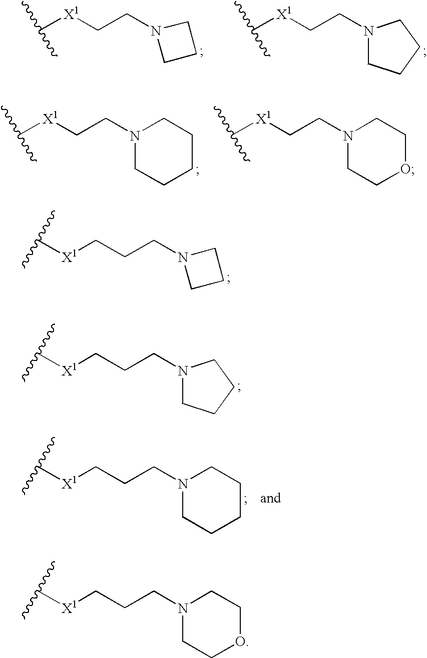

4. The compound according to claim 1, wherein the group of formula (c) is selected from the group consisting of: 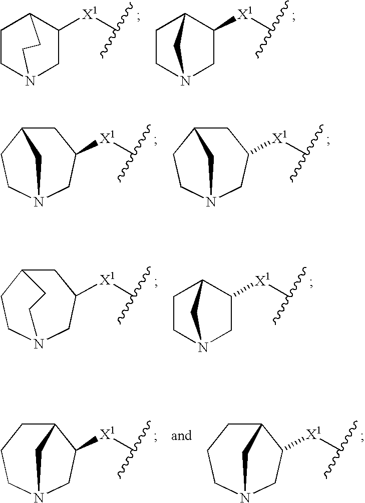
wherein X¹ is as defined in claim 1, and enantiomers thereof.

5. The compound according to claim 1, wherein one of A or B is a group of formula (c) wherein l is 0 and m is 2, and Y¹ is —O—, —S—, or —N(R¹¹)—, then Y² is not a bond.

6. The compound according to claim 1, wherein the group of formula (d) is selected from the group consisting of: 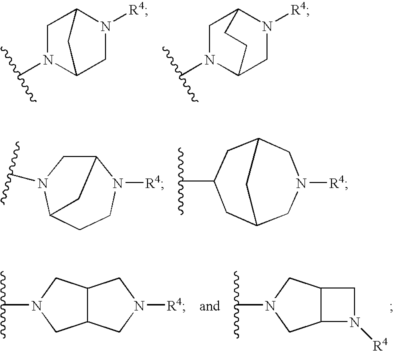
wherein R⁴ is as defined in claim 1, and enantiomers thereof.

7. The compound according to claim 1, wherein one of A or B is selected from a group of formula (d) and the other is selected from amino, dialkylamino, and acylamino.

8. The compound according to claim 1, wherein the group of formula (d) is 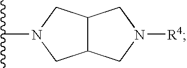
wherein R⁴ is hydrogen or alkyl.

9. The compound according to claim 1, wherein the group of formula (e) is selected from the group consisting of: 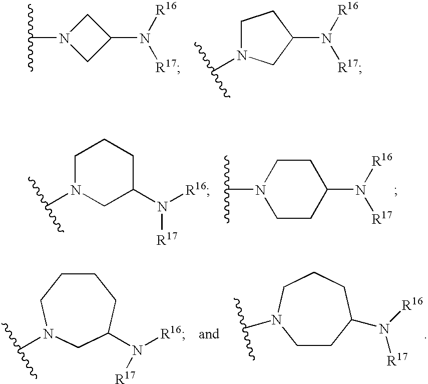
wherein R¹⁶ and R¹⁷ are as defined in claim 1, and enantiomers thereof.

10. The compound according to claim 1, wherein the group of formula (e is selected from the group consisting of: 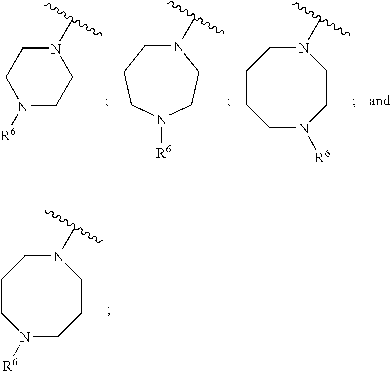
wherein R⁶ is hydrogen or alkyl, and enantiomers thereof.

11. The compound according to claim 1, wherein the group of formula (g) is selected from the group consisting of: 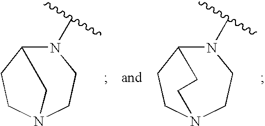
and enantiomers thereof.

12. The compound according to claim 1, selected from the group consisting of:
2,7-bis-[(3R)-1-azabicyclo[2.2.2]octan-3-yloxy]-fluoren-9-one;
2,7-bis[(3S)-1-azabicyclo[2.2.2]octan-3-yloxy]-fluoren-9-one;
2-[(3R)-1-azabicyclo[2.2.2]octan-3-yloxy]-fluoren-9-one;
2-[(3S)-1-azabicyclo[2.2.2]octan-3-yloxy]-fluoren-9-one;
2,7-bis(4-methyl-[1,4]diazepan-1-yl)-fluoren-9-one;
2,7-bis[3,7-diazabicyclo[3.3.0]octan-3-yl]-fluoren-9-one;
2,7-bis[7-methyl-3,7-diazabicyclo[3.3.0]octan-3-yl]fluoren-9-one;
2-[3,7-diazabicyclo[3.3.0]octan-3-yl]-fluoren-9-one;
2-[7-methyl-3,7-diazabicyclo[3.3.0]octan-3-yl]-fluoren-9-one;
2,7-bis(3-diethylamino-propyn-1-yl)-fluoren-9-one;
3,7-bis[(3S)-1-azabicyclo[2.2.2]octan-3-yloxy]-dibenzothiophene;
2-[(1S,5S)-3,6-diazabicyclo[3.2.0]heptan-3-yl]-dibenzothiophene-5,5-dioxide;
2-amino-7-[3,7-diazabicyclo[3.3.0]octan-3-yl]-fluoren-9-one;
2-[(3R)-1-azabicyclo[2.2.2]octan-3-yloxy]-xanthen-9-one;
2-(1-azabicyclo[2.2.2]octan-3-yloxy)-9H-carbazole;
2-(3,7-diazabicyclo[3.3.0]octan-3-yl)-7-methylamino-fluoren-9-one;
2-(3,7-diazabicyclo[3.3.0]octan-3-yl)-7-dimethylamino-fluoren-9-one;
2-amino-7-(7-methyl-3,7-diazabicyclo[3.3.0]octan-3-yl)-fluoren-9-one;
2-methylamino-7-(7-methyl-3,7-diazabicyclo[3.3.0]octan-3-yl)-fluoren-9-one;
2-dimethylamino-7-(7-methyl-3,7-diazabicyclo[3.3.0]octan-3-yl)-fluoren-9-one;
2-(3,7-diazabicyclo[3.3.0]octan-3-yl)-xanthen-9-one;
2-(7-methyl-3,7-diazabicyclo[3.3.0]octan-3-yl)-xanthen-9-one;
2-amino-7-(3,7-diazabicyclo[3.3.0]octan-3-yl)-xanthen-9-one;
2-amino-7-(7-methyl-3,7-diazabicyclo[3.3.0]octan-3-yl)-xanthen-9-one;
2-(3,7-diazabicyclo[3.3.0]octan-3-yl)-7-methylamino-xanthen-9-one;
2-methylamino-7-(7-methyl-3,7-diazabicyclo[3.3.0]octan-3-yl)-xanthen-9-one;
2-(3,7-diazabicyclo[3.3.0]octan-3-yl)-7-dimethylamino-xanthen-9-one;
2-dimethylamino-7-(7-methyl-3,7-diazabicyclo[3.3.0]octan-3-yl)-xanthen-9-one;
2-amino-7-[(3R)-1-azabicyclo[2.2.2]octan-3-yloxy]-fluoren-9-one;
2-amino-7-[(3S)-1-azabicyclo[2.2.2]octan-3-yloxy]-fluoren-9-one;
2-[(3R)-1-azabicyclo[2.2.2]octan-3-yloxy]-7-methylamino-fluoren-9-one;
2-[(3S)-1-azabicyclo[2.2.2]octan-3-yloxy]-7-methylamino-fluoren-9-one;
2-[(3R)-1-azabicyclo[2.2.2]octan-3-yloxy]-7-dimethylamino-fluoren-9-one;
2-[(3S)-1-azabicyclo[2.2.2]octan-3-yloxy]-7-dimethylamino-fluoren-9-one;
3,7-bis[(3R)-1-azabicyclo[2.2.2]octan-3-yloxy]-dibenzothiophene;
3,7-bis[(3S)-1-azabicyclo[2.2.2]octan-3-yloxy]-dibenzothiophene-5,5-dioxide;
3,7-bis[(3R)-1-azabicyclo[2.2.2]octan-3-yloxy]-dibenzothiophene-5,5-dioxide;
3,7-bis[3,7-diazabicyclo[3.3.0]octan-3-yl]-dibenzothiophene-5,5-dioxide;
3,7-bis[7-methyl-3,7-diazabicyclo[3.3.0]octan-3-yl]-dibenzothiophene-5,5-dioxide;
3-[3,7-diazabicyclo[3.3.0]octan-3-yl]-dibenzothiophene-5,5-dioxide;
3-[7-methyl-3,7-diazabicyclo[3.3.0]octan-3-yl]-dibenzothiophene-5,5-dioxide;
3-amino-7-[3,7-diazabicyclo[3.3.0]octan-3-yl]-dibenzothiophene-5,5-dioxide; and
2-[(3S)-1-azabicyclo[2.2.2]octan-3-yloxy]-xanthen-9-one.

13. A pharmaceutical composition comprising a therapeutically effective amount of a compound of claim 1 in combination with a pharmaceutically acceptable carrier.

14. A method for treating or preventing a condition or disorder modulated by anα7 nicotinic acetylcholine receptor comprising the step of administering a compound of the formula (II): 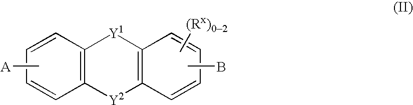
or a pharmaceutically acceptable salt, ester, amide, or prodrug thereof, wherein:
A and B are each independently selected from the group consisting of hydrogen; halogen; alkoxy; amino; alkylamino; acylamino; dialkylamino; cyano; nitro; and —SO₃H; and
a group of formula (a): 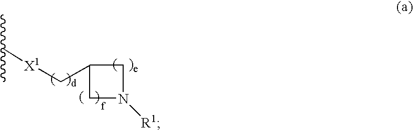
a group of formula (b): 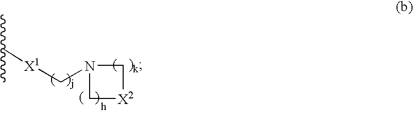
a group of formula (c): 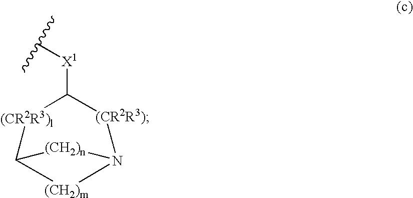
a group of formula (d): 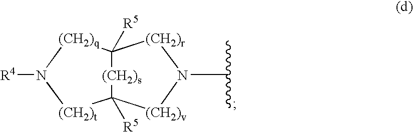
a group of formula (e): 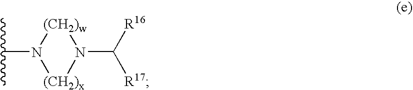
a group of formula (0: 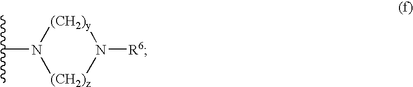
a group of formula (g): 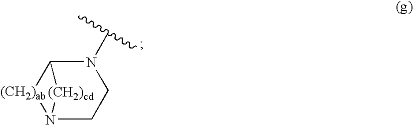
(h) —C≡CCH₂NR⁷R⁸; (i) —O—(C(R²⁰)₂₋₃N(R²¹)(R²²); and
(j) —O—(C(R²³)₂₋₃N⁺(R²⁴)(R²⁵)(R²⁶);
X¹ at each occurrence is selected from the group consisting of O, S, and —N(R⁹)—;
X² at each occurrence is selected from the group consisting of O, S, —CH₂—, and —N(R¹⁰)—;
Y¹ is independently selected from the group consisting of —C(O), —CH₂—, —CH(OH)—, —C(S)—, —N(R¹¹)—, —O—, —S—, —S(O)—, —S(O)₂—, —C(O)NH—, and —S(O)₂NH—;
Y² is a bond or Y² is independently selected from —O—, —S—, and —N(R¹²)—;
R¹ is independently selected from hydrogen and alkyl;
R² and R³ at each occurrence are each independently selected from the group consisting of hydrogen and alkyl;
R⁴ and R⁶ at each occurrence are each independently selected from the group consisting of hydrogen and alkyl;
R⁵ at each occurrence are each independently selected from the group consisting of hydrogen, alkyl, and alkoxycarbonyl;
R⁷ and R⁸ are each independently selected from hydrogen and alkyl or R⁷ and R⁸ taken together with the nitrogen atom to which each is attached form a 4- to 8-membered cyclic amine;
R⁹, R¹⁰, R¹¹, and R¹² at each occurrence are each independently selected from hydrogen and alkyl;
R¹⁶ and R¹⁷ are each independently selected from hydrogen and alkyl, or R¹⁶ and R¹⁷ taken together with the nitrogen atom to which each is attached form a 4 to 8-membered cyclic amine;
R²⁰ and R²³ are each independently selected from the group consisting of hydrogen and alkyl;
R²¹ and R²² are each independently selected from the group consisting of hydrogen and alkyl;
R²⁴, R²⁵, and R²⁶ are alkyl, or one pair of substituents selected from R²⁴, R²⁵, and R²⁶ is taken together with the nitrogen atom to which each is attached form a 4 to 8-membered cyclic amine and the remaining substituent is selected from hydrogen and alkyl;
R^{x} is independently selected at each occurrence from the group consisting of hydrogen, halogen, alkoxy, amino, alkylamino, dialkylamino, acylamino, dialkylaminoalkyl, and cyano;
d is independently selected from 0 or 1;
e and f are each independently selected from 0, 1, 2 or 3, provided that the sum total of e and f is 2, 3, or 4, provided that when d is 0, e and f are selected from 1, 2 or 3;
j is independently selected from 2 or 3;
h and k are each independently selected from 0, 1, or 2, provided that the sum total of h and k is 2, 3, or 4, provided that when X² is O, S, or N(R¹⁰), h and k are both 2;
l is 0 or 1, m is 2 or 3, and n is 0, 1, or 2, provided that the sum total of l, m, and n is 4, 5, or 6;
q, r, s, t, and v are each independently selected from 0, 1, or 2, provided that the sum of q and r; t and v; q, s, and t; and r, s, and v; are each at least 1;
w and x are each independently selected from 1, 2, or 3, provided that the sum total of w and x is 3, 4, 5, or 6;
y and z are each independently selected from 2, 3, or 4, provided that the sum total of y and z is 4, 5, or 6; and
ab is 2 or 3, and cd is 1 or 2.

15. The method according to claim 14, wherein the group A, the group B, or both groups A and B are selected from the group consisting of substituents (a)-(j).

16. The method according to claim 14, wherein the group A, the group B, or both groups A and B is group of formula (d).

17. The compound according to claim 14, wherein one of A or B is selected from a group of formula (d) and the other is selected from amino, dialkylamino, and acylamino.

18. The compound according to claim 14, wherein the group of formula (d) is 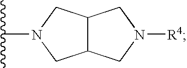
wherein R⁴ is hydrogen or alkyl.

19. The method according to claim 14, wherein the compound is selected from the group consisting of:
2,7-bis[(2R)-1-methylpyrrolidin-2-ylmethoxy]-fluoren-9-one;
2,7-bis[(2R)-azetidin-2-ylmethoxy]-fluoren-9-one;
2,7-bis[(2R)-1-methylazetidin-2-ylmethoxy]-fluoren-9-one;
2,7-bis[(3S)-pyrrolidin-3-yloxy]-fluoren-9-one;
2,7-bis[(3S)-1-methylpyrrolidin-3-yloxy]-fluoren-9-one;
2,7-bis-[(3R)-1-azabicyclo[2.2.2]octan-3-yloxy]-fluoren-9-one;
2,7-bis[(3S)-1-azabicyclo[2.2.2]octan-3-yloxy]-fluoren-9-one;
2-[(3R)-1-azabicyclo[2.2.2]octan-3-yloxy]-fluoren-9-one;
2-[(3S)-1-azabicyclo[2.2.2]octan-3-yloxy]-fluoren-9-one;
2,7-bis(4-methyl-[1,4]diazepan-1-yl)-fluoren-9-one;
2,7-bis[3,7-diazabicyclo[3.3.0]octan-3-yl]-fluoren-9-one;
2,7-bis[7-methyl-3,7-diazabicyclo[3.3.0]octan-3-yl]fluoren-9-one;
2-[3,7-diazabicyclo[3.3.0]octan-3-yl]-fluoren-9-one;
2-[7-methyl-3,7-diazabicyclo[3.3.0]octan-3-yl]-fluoren-9-one;
2,7-bis(3-diethylamino-propyn-1-yl)-fluoren-9-one;
3,7-bis(2-diethylaminoethoxy)dibenzothiophene;
3,7-bis(2-diethylaminoethoxy)dibenzothiophene-5-oxide;
3,7-bis[(3S)-1-azabicyclo[2.2.2]octan-3-yloxy]-dibenzothiophene;
2-[(1S,5S)-3,6-diazabicyclo[3.2.0]heptan-3-yl]-dibenzothiophene-5,5-dioxide;
2-amino-7-[3,7-diazabicyclo[3.3.0]octan-3-yl]-fluoren-9-one;
2-[(3R)-1-azabicyclo[2.2.2]octan-3-yloxy]-xanthen-9-one;
2-(1-azabicyclo[2.2.2]octan-3-yloxy)-9H-carbazole;
2,7-bis-(2-aminoethoxy)fluorene;
2,7-bis-(3-aminopropoxy)-fluorene;
2,7-bis-(2-methylaminoethoxy)-fluorene;
2,7-bis-(2-ethylaminoethoxy)-fluorene;
2,7-bis-(2-n-propylaminoethoxy)-fluorene;
2,7-bis-(3-methylaminopropoxy)-fluorene;
2,7-bis-(3-ethylaminopropoxy)-fluorene;
2,7-bis-(3-n-propylaminopropoxy)-fluorene;
2,7-bis-(2-dimethylaminoethoxy)-fluorene;
2,7-bis-(2-diethylaminoethoxy)-fluorene;
2,7-bis-(2-di-n-propylaminoethoxy)-fluorene;
2,7-bis-(3-dimethylaminopropoxy)-fluorene;
2,7-bis-(3-diethylaminopropoxy)-fluorene;
2,7-bis-(3-di-n-propylaminopropoxy)-fluorene;
2,7-bis-(2-azetidin-1-yl-ethoxy)-fluorene;
2,7-bis-(2-pyrrolidin-1-yl-ethoxy)-fluorene;
2,7-bis-(2-piperidin-1-yl-ethoxy)-fluorene;
2,7-bis-(3-azetidin-1-yl-propoxy)-fluorene;
2,7-bis-(3-pyrrolidin-1-yl-propoxy)-fluorene;
2,7-bis-(3-piperidin-1-yl-propoxy)-fluorene;
2,7-bis-(2-trimethylammoniumethoxy)-fluorene;
2,7-bis-(3-trimethylammoniumpropoxy)-fluorene;
2,6-bis-(2-aminoethoxy)-fluorene;
2,6-bis-(3-aminopropoxy)-fluorene;
2,6-bis-(2-methylaminoethoxy)-fluorene;
2,6-bis-(2-ethylaminoethoxy)-fluorene;
2,6-bis-(2-n-propylaminoethoxy)-fluorene;
2,6-bis-(3-methylaminopropoxy)-fluorene;
2,6-bis-(3-ethylaminopropoxy)-fluorene;
2,6-bis-(3-n-propylaminopropoxy)-fluorene;
2,6-bis-(2-dimethylaminoethoxy)-fluorene;
2,6-bis-(2-diethylaminoethoxy)-fluorene;
2,6-bis-(2-di-n-propylaminoethoxy)-fluorene;
2,6-bis-(3-dimethylaminopropoxy)-fluorene;
2,6-bis-(3-diethylaminopropoxy)-fluorene;
2,6-bis-(3-di-n-propylaminopropoxy)-fluorene;
2,6-bis-(2-azetidin-1-yl-ethoxy)-fluorene;
2,6-bis-(2-pyrrolidin-1-yl-ethoxy)-fluorene;
2,6-bis-(2-piperidin-1-yl-ethoxy)-fluorene;
2,6-bis-(3-azetidin-1-yl-propoxy)-fluorene;
2,6-bis-(3-pyrrolidin-1-yl-propoxy)-fluorene;
2,6-bis-(3-piperidin-1-yl-propoxy)-fluorene;
2,6-bis-(2-trimethylammoniumethoxy)-fluorene;
2,6-bis-(3-trimethylammoniumpropoxy)-fluorene;
3,6-bis-(2-aminoethoxy)fluorene;
3,6-bis-(3-aminopropoxy)fluorene;
3,6-bis-(2-methylaminoethoxy)-fluorene;
3,6-bis-(2-ethylaminoethoxy)-fluorene;
3,6-bis-(2-n-propylaminoethoxy)-fluorene;
3,6-bis-(3-methylaminopropoxy)-fluorene;
3,6-bis-(3-ethylaminopropoxy)-fluorene;
3,6-bis-(3-n-propylaminopropoxy)-fluorene;
3,6-bis-(2-dimethylaminoethoxy)-fluorene;
3,6-bis-(2-diethylaminoethoxy)-fluorene;
3,6-bis-(2-di-n-propylaminoethoxy)-fluorene;
3,6-bis-(3-dimethylaminopropoxy)-fluorene;
3,6-bis-(3-diethylaminopropoxy)-fluorene;
3,6-bis-(3-di-n-propylaminopropoxy)-fluorene;
3,6-bis-(2-azetidin-1-yl-ethoxy)-fluorene;
3,6-bis-(2-pyrrolidin-1-yl-ethoxy)-fluorene;
3,6-bis-(2-piperidin-1-yl-ethoxy)-fluorene;
3,6-bis-(3-azetidin-1-yl-propoxy)-fluorene;
3,6-bis-(3-pyrrolidin-1-yl-propoxy)-fluorene;
3,6-bis-(3-piperidin-1-yl-propoxy)-fluorene;
3,6-bis-(2-trimethylammoniumethoxy)-fluorene;
3,6-bis-(3-trimethylammoniumpropoxy)-fluorene;
2,7-bis-(2-aminoethoxy)fluoren-ol;
2,7-bis-(3-aminopropoxy)fluoren-9-ol;
2,7-bis-(2-methylaminoethoxy)-fluoren-9-ol;
2,7-bis-(2-ethylaminoethoxy)-fluoren-9-ol;
2,7-bis-(2-n-propylaminoethoxy)-fluoren-9-ol;
2,7-bis-(3-methylaminopropoxy)-fluoren-9-ol;
2,7-bis-(3-ethylaminopropoxy)-fluoren-9-ol;
2,7-bis-(3-n-propylaminopropoxy)-fluoren-9-ol;
2,7-bis-(2-dimethylaminoethoxy)-fluoren-9-ol;
2,7-bis-(2-diethylaminoethoxy)-fluoren-9-ol;
2,7-bis-(2-di-n-propylaminoethoxy)-fluoren-9-ol;
2,7-bis-(3-dimethylaminopropoxy)-fluoren-9-ol;
2,7-bis-(3-diethylaminopropoxy)-fluoren-9-ol;
2,7-bis-(3-di-n-propylaminopropoxy)-fluoren-9-ol;
2,7-bis-(2-azetidin-1-yl-ethoxy)-fluoren-9-ol;
2,7-bis-(2-pyrrolidin-1-yl-ethoxy)-fluoren-9-ol;
2,7-bis-(2-piperidin-1-yl-ethoxy)-fluoren-9-ol;
2,7-bis-(3-azetidin-1-yl-propoxy)-fluoren-9-ol;
2,7-bis-(3-pyrrolidin-1-yl-propoxy)-fluoren-9-ol;
2,7-bis-(3-piperidin-1-yl-propoxy)-fluoren-9-ol;
2,7-bis-(2-trimethylammoniumethoxy)-fluoren-9-ol;
2,7-bis-(3-trimethylammoniumpropoxy)-fluoren-9-ol;
2,6-bis-(2-aminoethoxy)-fluoren-9-ol;
2,6-bis-(3-aminopropoxy)-fluoren-9-ol;
2,6-bis-(2-methylaminoethoxy)-fluoren-9-ol;
2,6-bis-(2-ethylaminoethoxy)-fluoren-9-ol;
2,6-bis-(2-n-propylaminoethoxy)-fluoren-9-ol;
2,6-bis-(3-methylaminopropoxy)-fluoren-9-ol;
2,6-bis-(3-ethylaminopropoxy)-fluoren-9-ol;
2,6-bis-(3-n-propylaminopropoxy)-fluoren-9-ol;
2,6-bis-(2-dimethylaminoethoxy)-fluoren-9-ol;
2,6-bis-(2-diethylaminoethoxy)-fluoren-9-ol;
2,6-bis-(2-di-n-propylaminoethoxy)-fluoren-9-ol;
2,6-bis-(3-dimethylaminopropoxy)-fluoren-9-ol;
2,6-bis-(3-diethylaminopropoxy)-fluoren-9-ol;
2,6-bis-(3-di-n-propylaminopropoxy)-fluoren-9-ol;
2,6-bis-(2-azetidin-1-yl-ethoxy)-fluoren-9-ol;
2,6-bis-(2-pyrrolidin-1-yl-ethoxy)-fluoren-9-ol;
2,6-bis-(2-piperidin-1-yl-ethoxy)-fluoren-9-ol;
2,6-bis-(3-azetidin-1-yl-propoxy)-fluoren-9-ol;
2,6-bis-(3-pyrrolidin-1-yl-propoxy)-fluoren-9-ol;
2,6-bis-(3-piperidin-1-yl-propoxy)-fluoren-9-ol;
2,6-bis-(2-trimethylammoniumethoxy)-fluoren-9-ol;
2,6-bis-(3-trimethylammoniumpropoxy)-fluoren-9-ol;
3,6-bis-(2-aminoethoxy)-fluoren-9-ol;
3,6-bis-(3-aminopropoxy)-fluoren-9-ol;
3,6-bis-(2-methylaminoethoxy)-fluoren-9-ol;
3,6-bis-(2-ethylaminoethoxy)-fluoren-9-ol;
3,6-bis-(2-n-propylaminoethoxy)-fluoren-9-ol;
3,6-bis-(3-methylaminopropoxy)-fluoren-9-ol;
3,6-bis-(3-ethylaminopropoxy)-fluoren-9-ol;
3,6-bis-(3-n-propylaminopropoxy)-fluoren-9-ol;
3,6-bis-(2-dimethylaminoethoxy)-fluoren-9-ol;
3,6-bis-(2-diethylaminoethoxy)-fluoren-9-ol;
3,6-bis-(2-di-n-propylaminoethoxy)-fluoren-9-ol;
3,6-bis-(3-dimethylaminopropoxy)-fluoren-9-ol;
3,6-bis-(3-diethylaminopropoxy)-fluoren-9-ol;
3,6-bis-(3-di-n-propylaminopropoxy)-fluoren-9-ol;
3,6-bis-(2-azetidin-1-yl-ethoxy)-fluoren-9-ol;
3,6-bis-(2-pyrrolidin-1-yl-ethoxy)-fluoren-9-ol;
3,6-bis-(2-piperidin-1-yl-ethoxy)-fluoren-9-ol;
3,6-bis-(3-azetidin-1-yl-propoxy)-fluoren-9-ol;
3,6-bis-(3-pyrrolidin-1-yl-propoxy)-fluoren-9-ol;
3,6-bis-(3-piperidin-1-yl-propoxy)-fluoren-9-ol;
3,6-bis-(2-trimethylammoniumethoxy)-fluoren-9-ol;
3,6-bis-(3-trimethylammoniumpropoxy)-fluoren-9-ol;
2,7-bis-(2-aminoethoxy)-fluoren-9-one;
2,7-bis-(3-aminopropoxy)fluoren-9-one;
2,7-bis-(2-methylaminoethoxy)-fluoren-9-one;
2,7-bis-(2-ethylaminoethoxy)-fluoren-9-one;
2,7-bis-(2-n-propylaminoethoxy)-fluoren-9-one;
2,7-bis-(3-methylaminopropoxy)-fluoren-9-one;
2,7-bis-(3-ethylaminopropoxy)-fluoren-9-one;
2,7-bis-(3-n-propylaminopropoxy)-fluoren-9-one;
2,7-bis-(2-dimethylaminoethoxy)-fluoren-9-one;
2,7-bis-(2-diethylaminoethoxy)-fluoren-9-one;
2,7-bis-(2-di-n-propylaminoethoxy)-fluoren-9-one;
2,7-bis-(3-dimethylaminopropoxy)-fluoren-9-one;
2,7-bis-(3-diethylaminopropoxy)-fluoren-9-one;
2,7-bis-(3-di-n-propylaminopropoxy)-fluoren-9-one;
2,7-bis-(2-azetidin-1-yl-ethoxy)-fluoren-9-one;
2,7-bis-(2-pyrrolidin-1-yl-ethoxy)-fluoren-9-one;
2,7-bis-(2-piperidin-1-yl-ethoxy)-fluoren-9-one;
2,7-bis-(3-azetidin-1-yl-propoxy)-fluoren-9-one;
2,7-bis-(3-pyrrolidin-1-yl-propoxy)-fluoren-9-one;
2,7-bis-(3-piperidin-1-yl-propoxy)-fluoren-9-one;
2,7-bis-(2-trimethylammoniumethoxy)-fluoren-9-one;
2,7-bis-(3-trimethylammoniumpropoxy)-fluoren-9-one;
2,6-bis-(2-aminoethoxy)-fluoren-9-one;
2,6-bis-(3-aminopropoxy)-fluoren-9-one;
2,6-bis-(2-methylaminoethoxy)-fluoren-9-one;
2,6-bis-(2-ethylaminoethoxy)-fluoren-9-one;
2,6-bis-(2-n-propylaminoethoxy)-fluoren-9-one;
2,6-bis-(3-methylaminopropoxy)-fluoren-9-one;
2,6-bis-(3-ethylaminopropoxy)-fluoren-9-one;
2,6-bis-(3-n-propylaminopropoxy)-fluoren-9-one;
2,6-bis-(2-dimethylaminoethoxy)-fluoren-9-one;
2,6-bis-(2-diethylaminoethoxy)-fluoren-9-one;
2,6-bis-(2-di-n-propylaminoethoxy)-fluoren-9-one;
2,6-bis-(3-dimethylaminopropoxy)-fluoren-9-one;
2,6-bis-(3-diethylaminopropoxy)-fluoren-9-one;
2,6-bis-(3-di-n-propylaminopropoxy)-fluoren-9-one;
2,6-bis-(2-azetidin-1-yl-ethoxy)-fluoren-9-one;
2,6-bis-(2-pyrrolidin-1-yl-ethoxy)-fluoren-9-one;
2,6-bis-(2-piperidin-1-yl-ethoxy)-fluoren-9-one;
2,6-bis-(3-azetidin-1-yl-propoxy)-fluoren-9-one;
2,6-bis-(3-pyrrolidin-1-yl-propoxy)-fluoren-9-one;
2,6-bis-(3-piperidin-1-yl-propoxy)-fluoren-9-one;
2,6-bis-(2-trimethylammoniumethoxy)-fluoren-9-one;
2,6-bis-(3-trimethylammoniumpropoxy)-fluoren-9-one;
3,6-bis-(2-aminoethoxy)fluoren-9-one;
3,6-bis-(3-aminopropoxy)-fluoren-9-one;
3,6-bis-(2-methylaminoethoxy)-fluoren-9-one;
3,6-bis-(2-ethylaminoethoxy)-fluoren-9-one;
3,6-bis-(2-n-propylaminoethoxy)-fluoren-9-one;
3,6-bis-(3-methylaminopropoxy)-fluoren-9-one;
3,6-bis-(3-ethylaminopropoxy)-fluoren-9-one;
3,6-bis-(3-n-propylaminopropoxy)-fluoren-9-one;
3,6-bis-(2-dimethylaminoethoxy)-fluoren-9-one;
3,6-bis-(2-diethylaminoethoxy)-fluoren-9-one;
3,6-bis-(2-di-n-propylaminoethoxy)-fluoren-9-one;
3,6-bis-(3-dimethylaminopropoxy)-fluoren-9-one;
3,6-bis-(3-diethylaminopropoxy)-fluoren-9-one;
3,6-bis-(3-di-n-propylaminopropoxy)-fluoren-9-one;
3,6-bis-(2-azetidin-1-yl-ethoxy)-fluoren-9-one;
3,6-bis-(2-pyrrolidin-1-yl-ethoxy)-fluoren-9-one;
3,6-bis-(2-piperidin-1-yl-ethoxy)-fluoren-9-one;
3,6-bis-(3-azetidin-1-yl-propoxy)-fluoren-9-one;
3,6-bis-(3-pyrrolidin-1-yl-propoxy)-fluoren-9-one;
3,6-bis-(3-piperidin-1-yl-propoxy)-fluoren-9-one;
3,6-bis-(2-trimethylammoniumethoxy)-fluoren-9-one;
3,6-bis-(3-trimethylammoniumpropoxy)-fluoren-9-one;
3,7-bis-(2-aminoethoxy)-dibenzofuran;
3,7-bis-(3-aminopropoxy)-dibenzofuran;
3,7-bis-(2-methylaminoethoxy)-dibenzofuran;
3,7-bis-(2-ethylaminoethoxy)-dibenzofuran;
3,7-bis-(2-n-propylaminoethoxy)-dibenzofuran;
3,7-bis-(3-methylaminopropoxy)-dibenzofuran;
3,7-bis-(3-ethylaminopropoxy)-dibenzofuran;
3,7-bis-(3-n-propylaminopropoxy)-dibenzofuran;
3,7-bis-(2-dimethylaminoethoxy)-dibenzofuran;
3,7-bis-(2-diethylaminoethoxy)-dibenzofuran;
3,7-bis-(2-di-n-propylaminoethoxy)-dibenzofuran;
3,7-bis-(3-dimethylaminopropoxy)-dibenzofuran;
3,7-bis-(3-diethylaminopropoxy)-dibenzofuran;
3,7-bis-(3-di-n-propylaminopropoxy)-dibenzofuran;
3,7-bis-(2-azetidin-1-yl-ethoxy)-dibenzofuran;
3,7-bis-(2-pyrrolidin-1-yl-ethoxy)-dibenzofuran;
3,7-bis-(2-piperidin-1-yl-ethoxy)-dibenzofuran;
3,7-bis-(3-azetidin-1-yl-propoxy)-dibenzofuran;
3,7-bis-(3-pyrrolidin-1-yl-propoxy)-dibenzofuran;
3,7-bis-(3-piperidin-1-yl-propoxy)-dibenzofuran;
3,7-bis-(2-trimethylammoniumethoxy)-dibenzofuran;
3,7-bis-(3-trimethylammoniumpropoxy)-dibenzofuran;
2,7-bis-(2-aminoethoxy)-dibenzofuran;
2,7-bis-(3-aminopropoxy)-dibenzofuran;
2,7-bis-(2-methylaminoethoxy)-dibenzofuran;
2,7-bis-(2-ethylaminoethoxy)-dibenzofuran;
2,7-bis-(2-n-propylaminoethoxy)-dibenzofuran;
2,7-bis-(3-methylaminopropoxy)-dibenzofuran;
2,7-bis-(3-ethylaminopropoxy)-dibenzofuran;
2,7-bis-(3-n-propylaminopropoxy)-dibenzofuran;
2,7-bis-(2-dimethylaminoethoxy)-dibenzofuran;
2,7-bis-(2-diethylaminoethoxy)-dibenzofuran;
2,7-bis-(2-di-n-propylaminoethoxy)-dibenzofuran;
2,7-bis-(3-dimethylaminopropoxy)-dibenzofuran;
2,7-bis-(3-diethylaminopropoxy)-dibenzofuran;
2,7-bis-(3-di-n-propylaminopropoxy)-dibenzofuran;
2,7-bis-(2-azetidin-1-yl-ethoxy)-dibenzofuran;
2,7-bis-(2-pyrrolidin-1-yl-ethoxy)-dibenzofuran;
2,7-bis-(2-piperidin-1-yl-ethoxy)-dibenzofuran;
2,7-bis-(3-azetidin-1-yl-propoxy)-dibenzofuran;
2,7-bis-(3-pyrrolidin-1-yl-propoxy)-dibenzofuran;
2,7-bis-(3-piperidin-1-yl-propoxy)-dibenzofuran;
2,7-bis-(2-trimethylammoniumethoxy)-dibenzofuran;
2,7-bis-(3-trimethylammoniumpropoxy)-dibenzofuran;
2,8-bis-(2-aminoethoxy)-dibenzofuran;
2,8-bis-(3-aminopropoxy)-dibenzofuran;
2,8-bis-(2-methylaminoethoxy)-dibenzofuran;
2,8-bis-(2-ethylaminoethoxy)-dibenzofuran;
2,8-bis-(2-n-propylaminoethoxy)-dibenzofuran;
2,8-bis-(3-methylaminopropoxy)-dibenzofuran;
2,8-bis-(3-ethylaminopropoxy)-dibenzofuran;
2,8-bis-(3-n-propylaminopropoxy)-dibenzofuran;
2,8-bis-(2-dimethylaminoethoxy)-dibenzofuran;
2,8-bis-(2-diethylaminoethoxy)-dibenzofuran;
2,8-bis-(2-di-n-propylaminoethoxy)-dibenzofuran;
2,8-bis-(3-dimethylaminopropoxy)-dibenzofuran;
2,8-bis-(3-diethylaminopropoxy)-dibenzofuran;
2,8-bis-(3-di-n-propylaminopropoxy)-dibenzofuran;
2,8-bis-(2-azetidin-1-yl-ethoxy)-dibenzofuran;
2,8-bis-(2-pyrrolidin-1-yl-ethoxy)-dibenzofuran;
2,8-bis-(2-piperidin-1-yl-ethoxy)-dibenzofuran;
2,8-bis-(3-azetidin-1-yl-propoxy)-dibenzofuran;
2,8-bis-(3-pyrrolidin-1-yl-propoxy)-dibenzofuran;
2,8-bis-(3-piperidin-1-yl-propoxy)-dibenzofuran;
2,8-bis-(2-trimethylammoniumethoxy)-dibenzofuran;
2,8-bis-(3-trimethylammoniumpropoxy)-dibenzofuran;
3,7-bis-(2-aminoethoxy)-dibenzothiophene;
3,7-bis-(3-aminopropoxy)-dibenzothiophene;
3,7-bis-(2-methylaminoethoxy)-dibenzothiophene;
3,7-bis-(2-ethylaminoethoxy)-dibenzothiophene;
3,7-bis-(2-n-propylaminoethoxy)-dibenzothiophene;
3,7-bis-(3-methylaminopropoxy)-dibenzothiophene;
3,7-bis-(3-ethylaminopropoxy)-dibenzothiophene;
3,7-bis-(3-n-propylaminopropoxy)-dibenzothiophene;
3,7-bis-(2-dimethylaminoethoxy)-dibenzothiophene;
3,7-bis-(2-diethylaminoethoxy)-dibenzothiophene;
3,7-bis-(2-di-n-propylaminoethoxy)-dibenzothiophene;
3,7-bis-(3-dimethylaminopropoxy)-dibenzothiophene;
3,7-bis-(3-diethylaminopropoxy)-dibenzothiophene;
3,7-bis-(3-di-n-propylaminopropoxy)-dibenzothiophene;
3,7-bis-(2-azetidin-1-yl-ethoxy)-dibenzothiophene;
3,7-bis-(2-pyrrolidin-1-yl-ethoxy)-dibenzothiophene;
3,7-bis-(2-piperidin-1-yl-ethoxy)-dibenzothiophene;
3,7-bis-(3-azetidin-1-yl-propoxy)-dibenzothiophene;
3,7-bis-(3-pyrrolidin-1-yl-propoxy)-dibenzothiophene;
3,7-bis-(3-piperidin-1-yl-propoxy)-dibenzothiophene;
3,7-bis-(2-trimethylammoniumethoxy)-dibenzothiophene;
3,7-bis-(3-trimethylammoniumpropoxy)-dibenzothiophene;
2,7-bis-(2-aminoethoxy)-dibenzothiophene;
2,7-bis-(3-aminopropoxy)-dibenzothiophene;
2,7-bis-(2-methylaminoethoxy)-dibenzothiophene;
2,7-bis-(2-ethylaminoethoxy)-dibenzothiophene;
2,7-bis-(2-n-propylaminoethoxy)-dibenzothiophene;
2,7-bis-(3-methylaminopropoxy)-dibenzothiophene;
2,7-bis-(3-ethylaminopropoxy)-dibenzothiophene;
2,7-bis-(3-n-propylaminopropoxy)-dibenzothiophene;
2,7-bis-(2-dimethylaminoethoxy)-dibenzothiophene;
2,7-bis-(2-diethylaminoethoxy)-dibenzothiophene;
2,7-bis-(2-di-n-propylaminoethoxy)-dibenzothiophene;
2,7-bis-(3-dimethylaminopropoxy)-dibenzothiophene;
2,7-bis-(3-diethylaminopropoxy)-dibenzothiophene;
2,7-bis-(3-di-n-propylaminopropoxy)-dibenzothiophene;
2,7-bis-(2-azetidin-1-yl-ethoxy)-dibenzothiophene;
2,7-bis-(2-pyrrolidin-1-yl-ethoxy)-dibenzothiophene;
2,7-bis-(2-piperidin-1-yl-ethoxy)-dibenzothiophene;
2,7-bis-(3-azetidin-1-yl-propoxy)-dibenzothiophene;
2,7-bis-(3-pyrrolidin-1-yl-propoxy)-dibenzothiophene;
2,7-bis-(3-piperidin-1-yl-propoxy)-dibenzothiophene;
2,7-bis-(2-trimethylammoniumethoxy)-dibenzothiophene;
2,7-bis-(3-trimethylammoniumpropoxy)-dibenzothiophene;
2,8-bis-(2-aminoethoxy)-dibenzothiophene;
2,8-bis-(3-aminopropoxy)-dibenzothiophene;
2,8-bis-(2-methylaminoethoxy)-dibenzothiophene;
2,8-bis-(2-ethylaminoethoxy)-dibenzothiophene;
2,8-bis-(2-n-propylaminoethoxy)-dibenzothiophene;
2,8-bis-(3-methylaminopropoxy)-dibenzothiophene;
2,8-bis-(3-ethylaminopropoxy)-dibenzothiophene;
2,8-bis-(3-n-propylaminopropoxy)-dibenzothiophene;
2,8-bis-(2-dimethylaminoethoxy)-dibenzothiophene;
2,8-bis-(2-diethylaminoethoxy)-dibenzothiophene;
2,8-bis-(2-di-n-propylaminoethoxy)-dibenzothiophene;
2,8-bis-(3-dimethylaminopropoxy)-dibenzothiophene;
2,8-bis-(3-diethylaminopropoxy)-dibenzothiophene;
2,8-bis-(3-di-n-propylaminopropoxy)-dibenzothiophene;
2,8-bis-(2-azetidin-1-yl-ethoxy)-dibenzothiophene;
2,8-bis-(2-pyrrolidin-1-yl-ethoxy)-dibenzothiophene;
2,8-bis-(2-piperidin-1-yl-ethoxy)-dibenzothiophene;
2,8-bis-(3-azetidin-1-yl-propoxy)-dibenzothiophene;
2,8-bis-(3-pyrrolidin-1-yl-propoxy)-dibenzothiophene;
2,8-bis-(3-piperidin-1-yl-propoxy)-dibenzothiophene;
2,8-bis-(2-trimethylammoniumethoxy)-dibenzothiophene;
2,8-bis-(3-trimethylammoniumpropoxy)-dibenzothiophene;
3,7-bis-(2-aminoethoxy)-dibenzothiophene-5-oxide;
3,7-bis-(3-aminopropoxy)-dibenzothiophene-5-oxide;
3,7-bis-(2-methylaminoethoxy)-dibenzothiophene-5-oxide;
3,7-bis-(2-ethylaminoethoxy)-dibenzothiophene-5-oxide;
3,7-bis-(2-n-propylaminoethoxy)-dibenzothiophene-5-oxide;
3,7-bis-(3-methylaminopropoxy)-dibenzothiophene-5-oxide;
3,7-bis-(3-ethylaminopropoxy)-dibenzothiophene-5-oxide;
3,7-bis-(3-n-propylaminopropoxy)-dibenzothiophene-5-oxide;
3,7-bis-(2-dimethylaminoethoxy)-dibenzothiophene-5-oxide;
3,7-bis-(2-diethylaminoethoxy)-dibenzothiophene-5-oxide;
3,7-bis-(2-di-n-propylaminoethoxy)-dibenzothiophene-5-oxide;
3,7-bis-(3-dimethylaminopropoxy)-dibenzothiophene-5-oxide;
3,7-bis-(3-diethylaminopropoxy)-dibenzothiophene-5-oxide;
3,7-bis-(3-di-n-propylaminopropoxy)-dibenzothiophene-5-oxide;
3,7-bis-(2-azetidin-1-yl-ethoxy)-dibenzothiophene-5-oxide;
3,7-bis-(2-pyrrolidin-1-yl-ethoxy)-dibenzothiophene-5-oxide;
3,7-bis-(2-piperidin-1-yl-ethoxy)-dibenzothiophene-5-oxide;
3,7-bis-(3-azetidin-1-yl-propoxy)-dibenzothiophene-5-oxide;
3,7-bis-(3-pyrrolidin-1-yl-propoxy)-dibenzothiophene-5-oxide;
3,7-bis-(3-piperidin-1-yl-propoxy)-dibenzothiophene-5-oxide;
3,7-bis-(2-trimethylammoniumethoxy)-dibenzothiophene-5-oxide;
3,7-bis-(3-trimethylammoniumpropoxy)-dibenzothiophene-5-oxide;
2,7-bis-(2-aminoethoxy)-dibenzothiophene-5-oxide;
2,7-bis-(3-aminopropoxy)-dibenzothiophene-5-oxide;
2,7-bis-(2-methylaminoethoxy)-dibenzothiophene-5-oxide;
2,7-bis-(2-ethylaminoethoxy)-dibenzothiophene-5-oxide;
2,7-bis-(2-n-propylaminoethoxy)-dibenzothiophene-5-oxide;
2,7-bis-(3-methylaminopropoxy)-dibenzothiophene-5-oxide;
2,7-bis-(3-ethylaminopropoxy)-dibenzothiophene-5-oxide;
2,7-bis-(3-n-propylaminopropoxy)-dibenzothiophene-5-oxide;
2,7-bis-(2-dimethylaminoethoxy)-dibenzothiophene-5-oxide;
2,7-bis-(2-diethylaminoethoxy)-dibenzothiophene-5-oxide;
2,7-bis-(2-di-n-propylaminoethoxy)-dibenzothiophene-5-oxide;
2,7-bis-(3-dimethylaminopropoxy)-dibenzothiophene-5-oxide;
2,7-bis-(3-diethylaminopropoxy)-dibenzothiophene-5-oxide;
2,7-bis-(3-di-n-propylaminopropoxy)-dibenzothiophene-5-oxide;
2,7-bis-(2-azetidin-1-yl-ethoxy)-dibenzothiophene-5-oxide;
2,7-bis-(2-pyrrolidin-1-yl-ethoxy)-dibenzothiophene-5-oxide;
2,7-bis-(2-piperidin-1-yl-ethoxy)-dibenzothiophene-5-oxide;
2,7-bis-(3-azetidin-1-yl-propoxy)-dibenzothiophene-5-oxide;
2,7-bis-(3-pyrrolidin-1-yl-propoxy)-dibenzothiophene-5-oxide;
2,7-bis-(3-piperidin-1-yl-propoxy)-dibenzothiophene-5-oxide;
2,7-bis-(2-trimethylammoniumethoxy)-dibenzothiophene-5-oxide
2,7-bis-(3-trimethylammoniumpropoxy)-dibenzothiophene-5-oxide;
2,8-bis-(2-aminoethoxy)-dibenzothiophene-5-oxide;
2,8-bis-(3-aminopropoxy)-dibenzothiophene-5-oxide;
2,8-bis-(2-methylaminoethoxy)-dibenzothiophene-5-oxide;
2,8-bis-(2-ethylaminoethoxy)-dibenzothiophene-5-oxide;
2,8-bis-(2-n-propylaminoethoxy)-dibenzothiophene-5-oxide;
2,8-bis-(3-methylaminopropoxy)-dibenzothiophene-5-oxide;
2,8-bis-(3-ethylaminopropoxy)-dibenzothiophene-5-oxide;
2,8-bis-(3-n-propylaminopropoxy)-dibenzothiophene-5-oxide;
2,8-bis-(2-dimethylaminoethoxy)-dibenzothiophene-5-oxide;
2,8-bis-(2-diethylaminoethoxy)-dibenzothiophene-5-oxide;
2,8-bis-(2-di-n-propylaminoethoxy)-dibenzothiophene-5-oxide;
2,8-bis-(3-dimethylaminopropoxy)-dibenzothiophene-5-oxide;
2,8-bis-(3-diethylaminopropoxy)-dibenzothiophene-5-oxide;
2,8-bis-(3-di-n-propylaminopropoxy)-dibenzothiophene-5-oxide;
2,8-bis-(2-azetidin-1-yl-ethoxy)-dibenzothiophene-5-oxide;
2,8-bis-(2-pyrrolidin-1-yl-ethoxy)-dibenzothiophene-5-oxide;
2,8-bis-(2-piperidin-1-yl-ethoxy)-dibenzothiophene-5-oxide;
2,8-bis-(3-azetidin-1-yl-propoxy)-dibenzothiophene-5-oxide;
2,8-bis-(3-pyrrolidin-1-yl-propoxy)-dibenzothiophene-5-oxide;
2,8-bis-(3-piperidin-1-yl-propoxy)-dibenzothiophene-5-oxide;
2,8-bis-(2-trimethylammoniumethoxy)-dibenzothiophene-5-oxide;
2,8-bis-(3-trimethylammoniumpropoxy)-dibenzothiophene-5-oxide;
3,7-bis-(2-aminoethoxy)-dibenzothiophene-5,5-dioxide;
3,7-bis-(3-aminopropoxy)-dibenzothiophene-5,5-dioxide;
3,7-bis-(2-methylaminoethoxy)-dibenzothiophene-5,5-dioxide;
3,7-bis-(2-ethylaminoethoxy)-dibenzothiophene-5,5-dioxide;
3,7-bis-(2-n-propylaminoethoxy)-dibenzothiophene-5,5-dioxide;
3,7-bis-(3-methylaminopropoxy)-dibenzothiophene-5,5-dioxide;
3,7-bis-(3-ethylaminopropoxy)-dibenzothiophene-5,5-dioxide;
3,7-bis-(3-n-propylaminopropoxy)-dibenzothiophene-5,5-dioxide;
3,7-bis-(2-dimethylaminoethoxy)-dibenzothiophene-5,5-dioxide;
3,7-bis-(2-diethylaminoethoxy)-dibenzothiophene-5,5-dioxide;
3,7-bis-(2-di-n-propylaminoethoxy)-dibenzothiophene-5,5-dioxide;
3,7-bis-(3-dimethylaminopropoxy)-dibenzothiophene-5,5-dioxide;
3,7-bis-(3-diethylaminopropoxy)-dibenzothiophene-5,5-dioxide;
3,7-bis-(3-di-n-propylaminopropoxy)-dibenzothiophene-5,5-dioxide;
3,7-bis-(2-azetidin-1-yl-ethoxy)-dibenzothiophene-5,5-dioxide;
3,7-bis-(2-pyrrolidin-1-yl-ethoxy)-dibenzothiophene-5,5-dioxide;
3,7-bis-(2-piperidin-1-yl-ethoxy)-dibenzothiophene-55,5-dioxide;
3,7-bis-(3-azetidin-1-yl-propoxy)-dibenzothiophene-55,5-dioxide;
3,7-bis-(3-pyrrolidin-1-yl-propoxy)-dibenzothiophene-5,5-dioxide;
3,7-bis-(3-piperidin-1-yl-propoxy)-dibenzothiophene-5,5-dioxide;
3,7-bis-(2-trimethylammoniumethoxy)-dibenzothiophene-5,5-dioxide;
3,7-bis-(3-trimethylammoniumpropoxy)-dibenzothiophene-5,5-dioxide;
2,7-bis-(2-aminoethoxy)-dibenzothiophene-5,5-dioxide;
2,7-bis-(3-aminopropoxy)-dibenzothiophene-5,5-dioxide;
2,7-bis-(2-methylaminoethoxy)-dibenzothiophene-5,5-dioxide;
2,7-bis-(2-ethylaminoethoxy)-dibenzothiophene-5,5-dioxide;
2,7-bis-(2-n-propylaminoethoxy)-dibenzothiophene-5,5-dioxide;
2,7-bis-(3-methylaminopropoxy)-dibenzothiophene-5,5-dioxide;
2,7-bis-(3-ethylaminopropoxy)-dibenzothiophene-5,5-dioxide;
2,7-bis-(3-n-propylaminopropoxy)-dibenzothiophene-5,5-dioxide;
2,7-bis-(2-dimethylaminoethoxy)-dibenzothiophene-5,5-dioxide;
2,7-bis-(2-diethylaminoethoxy)-dibenzothiophene-5,5-dioxide;
2,7-bis-(2-di-n-propylaminoethoxy)-dibenzothiophene-5,5-dioxide;
2,7-bis-(3-dimethylaminopropoxy)-dibenzothiophene-5,5-dioxide;
2,7-bis-(3-diethylaminopropoxy)-dibenzothiophene-5,5-dioxide;
2,7-bis-(3-di-n-propylaminopropoxy)-dibenzothiophene-5,5-dioxide;
2,7-bis-(2-azetidin-1-yl-ethoxy)-dibenzothiophene-5,5-dioxide;
2,7-bis-(2-pyrrolidin-1-yl-ethoxy)-dibenzothiophene-5,5-dioxide;
2,7-bis-(2-piperidin-1-yl-ethoxy)-dibenzothiophene-5,5-dioxide;
2,7-bis-(3-azetidin-1-yl-propoxy)-dibenzothiophene-5,5-dioxide;
2,7-bis-(3-pyrrolidin-1-yl-propoxy)-dibenzothiophene-5,5-dioxide;
2,7-bis-(3-piperidin-1-yl-propoxy)-dibenzothiophene-5,5-dioxide;
2,7-bis-(2-trimethylammoniumethoxy)-dibenzothiophene-5,5-dioxide;
2,7-bis-(3-trimethylammoniumpropoxy)-dibenzothiophene-5,5-dioxide;
2,8-bis-(2-aminoethoxy)-dibenzothiophene-5,5-dioxide;
2,8-bis-(3-aminopropoxy)-dibenzothiophene-5,5-dioxide;
2,8-bis-(2-methylaminoethoxy)-dibenzothiophene-5,5-dioxide;
2,8-bis-(2-ethylaminoethoxy)-dibenzothiophene-5,5-dioxide;
2,8-bis-(2-n-propylaminoethoxy)-dibenzothiophene-5,5-dioxide;
2,8-bis-(3-methylaminopropoxy)-dibenzothiophene-5,5-dioxide;
2,8-bis-(3-ethylaminopropoxy)-dibenzothiophene-5,5-dioxide;
2,8-bis-(3-n-propylaminopropoxy)-dibenzothiophene-5,5-dioxide;
2,8-bis-(2-dimethylaminoethoxy)-dibenzothiophene-5,5-dioxide;
2,8-bis-(2-diethylaminoethoxy)-dibenzothiophene-5,5-dioxide;
2,8-bis-(2-di-n-propylaminoethoxy)-dibenzothiophene-5,5-dioxide;
2,8-bis-(3-dimethylaminopropoxy)-dibenzothiophene-5,5-dioxide;
2,8-bis-(3-diethylaminopropoxy)-dibenzothiophene-5,5-dioxide;
2,8-bis-(3-di-n-propylaminopropoxy)-dibenzothiophene-5,5-dioxide;
2,8-bis-(2-azetidin-1-yl-ethoxy)-dibenzothiophene-5,5-dioxide;
2,8-bis-(2-pyrrolidin-1-yl-ethoxy)-dibenzothiophene-5,5-dioxide;
2,8-bis-(2-piperidin-1-yl-ethoxy)-dibenzothiophene-5,5-dioxide;
2,8-bis-(3-azetidin-1-yl-propoxy)-dibenzothiophene-55,5-dioxide;
2,8-bis-(3-pyrrolidin-1-yl-propoxy)-dibenzothiophene-5,5-dioxide;
2,8-bis-(3-piperidin-1-yl-propoxy)-dibenzothiophene-5,5-dioxide;
2,8-bis-(2-methylammoniumethoxy)-dibenzothiophene-5,5-dioxide;
2,8-bis-(3-trimethylammoniumpropoxy)-dibenzothiophene-5,5-dioxide;
2,7-bis-(2-aminoethoxy)-9H-carbazole;
2,7-bis-(3-aminopropoxy)-9H-carbazole;
2,7-bis-(2-methylaminoethoxy)-9H-carbazole;
2,7-bis-(2-ethylaminoethoxy)-9H-carbazole;
2,7-bis-(2-n-propylaminoethoxy)-9H-carbazole;
2,7-bis-(3-methylaminopropoxy)-9H-carbazole;
2,7-bis-(3-ethylaminopropoxy)-9H-carbazole;
2,7-bis-(3-n-propylaminopropoxy)-9H-carbazole;
2,7-bis-(2-dimethylaminoethoxy)-9H-carbazole;
2,7-bis-(2-diethylaminoethoxy)-9H-carbazole;
2,7-bis-(2-di-n-propylaminoethoxy)-9H-carbazole;
2,7-bis-(3-dimethylaminopropoxy)-9H-carbazole;
2,7-bis-(3-diethylaminopropoxy)-9H-carbazole;
2,7-bis-(3-di-n-propylaminopropoxy)-9H-carbazole;
2,7-bis-(2-azetidin-1-yl-ethoxy)-9H-carbazole;
2,7-bis-(2-pyrrolidin-1-yl-ethoxy)-9H-carbazole;
2,7-bis-(2-piperidin-1-yl-ethoxy)-9H-carbazole;
2,7-bis-(3-azetidin-1-yl-propoxy)-9H-carbazole;
2,7-bis-(3-pyrrolidin-1-yl-propoxy)-9H-carbazole;
2,7-bis-(3-piperidin-1-yl-propoxy)-9H-carbazole;
2,7-bis-(2-trimethylammoniumethoxy)-9H-carbazole;
2,7-bis-(3-trimethylammoniumpropoxy)-9H-carbazole;
2,6-bis-(2-aminoethoxy)-9H-carbazole;
2,6-bis-(3-aminopropoxy)-9H-carbazole;
2,6-bis-(2-methylaminoethoxy)-9H-carbazole;
2,6-bis-(2-ethylaminoethoxy)-9H-carbazole;
2,6-bis-(2-n-propylaminoethoxy)-9H-carbazole;
2,6-bis-(3-methylaminopropoxy)-9H-carbazole;
2,6-bis-(3-ethylaminopropoxy)-9H-carbazole;
2,6-bis-(3-n-propylaminopropoxy)-9H-carbazole;
2,6-bis-(2-dimethylaminoethoxy)-9H-carbazole;
2,6-bis-(2-diethylaminoethoxy)-9H-carbazole;
2,6-bis-(2-di-n-propylaminoethoxy)-9H-carbazole;
2,6-bis-(3-dimethylaminopropoxy)-9H-carbazole;
2,6-bis-(3-diethylaminopropoxy)-9H-carbazole;
2,6-bis-(3-di-n-propylaminopropoxy)-9H-carbazole;
2,6-bis-(2-azetidin-1-yl-ethoxy)-9H-carbazole;
2,6-bis-(2-pyrrolidin-1-yl-ethoxy)-9H-carbazole;
2,6-bis-(2-piperidin-1-yl-ethoxy)-9H-carbazole;
2,6-bis-(3-azetidin-1-yl-propoxy)-9H-carbazole;
2,6-bis-(3-pyrrolidin-1-yl-propoxy)-9H-carbazole;
2,6-bis-(3-piperidin-1-yl-propoxy)-9H-carbazole;
2,6-bis-(2-trimethylammoniumethoxy)-9H-carbazole;
2,6-bis-(3-trimethylammoniumpropoxy)-9H-carbazole;
3,6-bis-(2-aminoethoxy)-9H-carbazole;
3,6-bis-(3-aminopropoxy)-9H-carbazole;
3,6-bis-(2-methylaminoethoxy)-9H-carbazole;
3,6-bis-(2-ethylaminoethoxy)-9H-carbazole;
3,6-bis-(2-n-propylaminoethoxy)-9H-carbazole;
3,6-bis-(3-methylaminopropoxy)-9H-carbazole;
3,6-bis-(3-ethylaminopropoxy)-9H-carbazole;
3,6-bis-(3-n-propylaminopropoxy)-9H-carbazole;
3,6-bis-(2-dimethylaminoethoxy)-9H-carbazole;
3,6-bis-(2-diethylaminoethoxy)-9H-carbazole;
3,6-bis-(2-di-n-propylaminoethoxy)-9H-carbazole;
3,6-bis-(3-dimethylaminopropoxy)-9H-carbazole;
3,6-bis-(3-diethylaminopropoxy)-9H-carbazole;
3,6-bis-(3-di-n-propylaminopropoxy)-9H-carbazole;
3,6-bis-(2-azetidin-1-yl-ethoxy)-9H-carbazole;
3,6-bis-(2-pyrrolidin-1-yl-ethoxy)-9H-carbazole;
3,6-bis-(2-piperidin-1-yl-ethoxy)-9H-carbazole;
3,6-bis-(3-azetidin-1-yl-propoxy)-9H-carbazole;
3,6-bis-(3-pyrrolidin-1-yl-propoxy)-9H-carbazole;
3,6-bis-(3-piperidin-1-yl-propoxy)-9H-carbazole;
3,6-bis-(2-trimethylammoniumethoxy)-9H-carbazole;
3,6-bis-(3-trimethylammoniumpropoxy)-9H-carbazole;
2,7-bis-(2-aminoethoxy)-9-methyl-9H-carbazole;
2,7-bis-(3-aminopropoxy)-9-methyl-9H-carbazole;
2,7-bis-(2-methylaminoethoxy)-9-methyl-9H-carbazole;
2,7-bis-(2-ethylaminoethoxy)-9-methyl-9H-carbazole;
2,7-bis-(2-n-propylaminoethoxy)-9-methyl-9H-carbazole;
2,7-bis-(3-methylaminopropoxy)-9-methyl-9H-carbazole;
2,7-bis-(3-ethylaminopropoxy)-9-methyl-9H-carbazole;
2,7-bis-(3-n-propylaminopropoxy)-9-methyl-9H-carbazole;
2,7-bis-(2-dimethylaminoethoxy)-9-methyl-9H-carbazole;
2,7-bis-(2-diethylaminoethoxy)-9-methyl-9H-carbazole;
2,7-bis-(2-di-n-propylaminoethoxy)-9-methyl-9H-carbazole;
2,7-bis-(3-dimethylaminopropoxy)-9-methyl-9H-carbazole;
2,7-bis-(3-diethylaminopropoxy)-9-methyl-9H-carbazole;
2,7-bis-(3-di-n-propylaminopropoxy)-9-methyl-9H-carbazole;
2,7-bis-(2-azetidin-1-yl-ethoxy)-9-methyl-9H-carbazole;
2,7-bis-(2-pyrrolidin-1-yl-ethoxy)-9-methyl-9H-carbazole;
2,7-bis-(2-piperidin-1-yl-ethoxy)-9-methyl-9H-carbazole;
2,7-bis-(3-azetidin-1-yl-propoxy)-9-methyl-9H-carbazole;
2,7-bis-(3-pyrrolidin-1-yl-propoxy)-9-methyl-9H-carbazole;
2,7-bis-(3-piperidin-1-yl-propoxy)-9-methyl-9H-carbazole;
2,7-bis-(2-trimethylammoniumethoxy)-9-methyl-9H-carbazole;
2,7-bis-(3-trimethylammoniumpropoxy)-9-methyl-9H-carbazole;
2,6-bis-(2-aminoethoxy)-9-methyl-9H-carbazole;
2,6-bis-(3-aminopropoxy)-9-methyl-9H-carbazole;
2,6-bis-(2-methylaminoethoxy)-9-methyl-9H-carbazole;
2,6-bis-(2-ethylaminoethoxy)-9-methyl-9H-carbazole;
2,6-bis-(2-n-propylaminoethoxy)-9-methyl-9H-carbazole;
2,6-bis-(3-methylaminopropoxy)-9-methyl-9H-carbazole;
2,6-bis-(3-ethylaminopropoxy)-9-methyl-9H-carbazole;
2,6-bis-(3-n-propylaminopropoxy)-9-methyl-9H-carbazole;
2,6-bis-(2-dimethylaminoethoxy)-9-methyl-9H-carbazole;
2,6-bis-(2-diethylaminoethoxy)-9-methyl-9H-carbazole;
2,6-bis-(2-di-n-propylaminoethoxy)-9-methyl-9H-carbazole;
2,6-bis-(3-dimethylaminopropoxy)-9-methyl-9H-carbazole;
2,6-bis-(3-diethylaminopropoxy)-9-methyl-9H-carbazole;
2,6-bis-(3-di-n-propylaminopropoxy)-9-methyl-9H-carbazole;
2,6-bis-(2-azetidin-1-yl-ethoxy)-9-methyl-9H-carbazole;
2,6-bis-(2-pyrrolidin-1-yl-ethoxy)-9-methyl-9H-carbazole;
2,6-bis-(2-piperidin-1-yl-ethoxy)-9-methyl-9H-carbazole;
2,6-bis-(3-azetidin-1-yl-propoxy)-9-methyl-9H-carbazole;
2,6-bis-(3-pyrrolidin-1-yl-propoxy)-Omethyl-9H-carbazole;
2,6-bis-(3-piperidin-1-yl-propoxy)-9-methyl-9H-carbazole;
2,6-bis-(2-methylammoniumethoxy)-9-methyl-9H-carbazole;
2,6-bis-(3-trimethylammoniumpropoxy)-9-methyl-9H-carbazole;
3,6-bis-(2-aminoethoxy)-9-methyl-9H-carbazole;
3,6-bis-(3-aminopropoxy)-9-methyl-9H-carbazole;
3,6-bis-(2-methylaminoethoxy)-9-methyl-9H-carbazole;
3,6-bis-(2-ethylaminoethoxy)-9-methyl-9H-carbazole;
3,6-bis-(2-n-propylaminoethoxy)-9-methyl-9H-carbazole;
3,6-bis-(3-methylaminopropoxy)-9-methyl-9H-carbazole;
3,6-bis-(3-ethylaminopropoxy)-9-methyl-9H-carbazole;
3,6-bis-(3-n-propylaminopropoxy)-9-methyl-9H-carbazole;
3,6-bis-(2-dimethylaminoethoxy)-9-methyl-9H-carbazole;
3,6-bis-(2-diethylaminoethoxy)-9-methyl-9H-carbazole;
3,6-bis-(2-di-n-propylaminoethoxy)-9-methyl-9H-carbazole;
3,6-bis-(3-dimethylaminopropoxy)-9-methyl-9H-carbazole;
3,6-bis-(3-diethylaminopropoxy)-9-methyl-9H-carbazole;
3,6-bis-(3-di-n-propylaminopropoxy)-9-methyl-9H-carbazole;
3,6-bis-(2-azetidin-1-yl-ethoxy)-9-methyl-9H-carbazole;
3,6-bis-(2-pyrrolidin-1-yl-ethoxy)-9-methyl-9H-carbazole;
3,6-bis-(2-piperidin-1-yl-ethoxy)-9-methyl-9H-carbazole;
3,6-bis-(3-azetidin-1-yl-propoxy)-9-methyl-9H-carbazole;
3,6-bis-(3-pyrrolidin-1-yl-propoxy)-9-methyl-9H-carbazole;
3,6-bis-(3-piperidin-1-yl-propoxy)-9-methyl-9H-carbazole;
3,6-bis-(2-trimethylammoniumethoxy)-9-methyl-9H-carbazole;
3,6-bis-(3-trimethylammoniumpropoxy)-9-methyl-9H-carbazole;
2,7-bis-(2-aminoethoxy)-xanthen-9-one;
2,7-bis-(3-aminopropoxy)-xanthen-9-one;
2,7-bis-(2-methylaminoethoxy)-xanthen-9-one;
2,7-bis-(2-ethylaminoethoxy)-xanthen-9-one;
2,7-bis-(2-n-propylaminoethoxy)-xanthen-9-one;
2,7-bis-(3-methylaminopropoxy)-xanthen-9-one;
2,7-bis-(3-ethylaminopropoxy)-xanthen-9-one;
2,7-bis-(3-n-propylaminopropoxy)-xanthen-9-one;
2,7-bis-(2-dimethylaminoethoxy)-xanthen-9-one;
2,7-bis-(2-diethylaminoethoxy)-xanthen-9-one;
2,7-bis-(2-di-n-propylaminoethoxy)-xanthen-9-one;
2,7-bis-(3-dimethylaminopropoxy)-xanthen-9-one;
2,7-bis-(3-diethylaminopropoxy)-xanthen-9-one;
2,7-bis-(3-di-n-propylaminopropoxy)-xanthen-9-one;
2,7-bis-(2-azetidin-1-yl-ethoxy)-xanthen-9-one;
2,7-bis-(2-pyrrolidin-1-yl-ethoxy)-xanthen-9-one;
2,7-bis-(2-piperidin-1-yl-ethoxy)-xanthen-9-one;
2,7-bis-(3-azetidin-1-yl-propoxy)-xanthen-9-one;
2,7-bis-(3-pyrrolidin-1-yl-propoxy)-xanthen-9-one;
2,7-bis-(3-piperidin-1-yl-propoxy)-xanthen-9-one;
2,7-bis-(2-trimethylammoniumethoxy)-xanthen-9-one;
2,7-bis-(3-trimethylammoniumpropoxy)-xanthen-9-one;
2,6-bis-(2-methylaminoethoxy)-xanthen-9-one;
2,6-bis-(2-ethylaminoethoxy)-xanthen-9-one;
2,6-bis-(2-n-propylaminoethoxy)-xanthen-9-one;
2,6-bis-(3-methylaminopropoxy)-xanthen-9-one;
2,6-bis-(3-ethylaminopropoxy)-xanthen-9-one;
2,6-bis-(3-n-propylaminopropoxy)-xanthen-9-one;
2,6-bis-(2-dimethylaminoethoxy)-xanthen-9-one;
2,6-bis-(2-diethylaminoethoxy)-xanthen-9-one;
2,6-bis-(2-di-n-propylaminoethoxy)-xanthen-9-one;
2,6-bis-(3-dimethylaminopropoxy)-xanthen-9-one;
2,6-bis-(3-diethylaminopropoxy)-xanthen-9-one;
2,6-bis-(3-di-n-propylaminopropoxy)-xanthen-9-one;
2,6-bis-(2-azetidin-1-yl-ethoxy)-xanthen-9-one;
2,6-bis-(2-pyrrolidin-1-yl-ethoxy)-xanthen-9-one;
2,6-bis-(2-piperidin-1-yl-ethoxy)-xanthen-9-one;
2,6-bis-(3-azetidin-1-yl-propoxy)-xanthen-9-one;
2,6-bis-(3-pyrrolidin-1-yl-propoxy)-xanthen-9-one;
2,6-bis-(3-piperidin-1-yl-propoxy)-xanthen-9-one;
2,6-bis-(2-trimethylammoniumethoxy)-xanthen-9-one;
2,6-bis-(3-trimethylammoniumpropoxy)-xanthen-9-one;
3,6-bis-(2-aminoethoxy)-xanthen-9-one;
3,6-bis-(3-aminopropoxy)-xanthen-9-one;
3,6-bis-(2-methylaminoethoxy)-xanthen-9-one;
3,6-bis-(2-ethylaminoethoxy)-xanthen-9-one;
3,6-bis-(2-n-propylaminoethoxy)-xanthen-9-one;
3,6-bis-(3-methylaminopropoxy)-xanthen-9-one;
3,6-bis-(3-ethylaminopropoxy)-xanthen-9-one;
3,6-bis-(3-n-propylaminopropoxy)-xanthen-9-one;
3,6-bis-(2-dimethylaminoethoxy)-xanthen-9-one;
3,6-bis-(2-diethylaminoethoxy)-xanthen-9-one;
3,6-bis-(2-di-n-propylaminoethoxy)-xanthen-9-one;
3,6-bis-(3-dimethylaminopropoxy)-xanthen-9-one;
3,6-bis-(3-diethylaminopropoxy)-xanthen-9-one;
3,6-bis-(3-di-n-propylaminopropoxy)-xanthen-9-one;
3,6-bis-(2-azetidin-1-yl-ethoxy)-xanthen-9-one;
3,6-bis-(2-pyrrolidin-1-yl-ethoxy)-xanthen-9-one;
3,6-bis-(2-piperidin-1-yl-ethoxy)-xanthen-9-one;
3,6-bis-(3-azetidin-1-yl-propoxy)-xanthen-9-one;
3,6-bis-(3-pyrrolidin-1-yl-propoxy)-xanthen-9-one;
3,6-bis-(3-piperidin-1-yl-propoxy)-xanthen-9-one;
3,6-bis-(2-trimethylammoniumethoxy)-xanthen-9 one;
3,6-bis-(3-trimethylammoniumpropoxy)-xanthen-9-one;
2,7-bis-(2-aminoethoxy)-thioxanthen-9-one;
2,7-bis-(3-aminopropoxy)-thioxanthen-9-one;
2,7-bis-(2-methylaminoethoxy)-thioxanthen-9-one;
2,7-bis-(2-ethylaminoethoxy)-thioxanthen-9-one;
2,7-bis-(2-n-propylaminoethoxy)-thioxanthen-9-one;
2,7-bis-(3-methylaminopropoxy)-thioxanthen-9-one;
2,7-bis-(3-ethylaminopropoxy)-thioxanthen-9-one;
2,7-bis-(3-n-propylaminopropoxy)-thioxanthen-9-one;
2,7-bis-(2-dimethylaminoethoxy)-thioxanthen-9-one;
2,7-bis-(2-diethylaminoethoxy)-thioxanthen-9-one;
2,7-bis-(2-di-n-propylaminoethoxy)-thioxanthen-9-one;
2,7-bis-(3-dimethylaminopropoxy)-thioxanthen-9-one;
2,7-bis-(3-diethylaminopropoxy)-thioxanthen-9-one;
2,7-bis-(3-di-n-propylaminopropoxy)-thioxanthen-9-one;
2,7-bis-(2-azetidin-1-yl-ethoxy)-thioxanthen-9-one;
2,7-bis-(2-pyrrolidin-1-yl-ethoxy)-thioxanthen-9-one;
2,7-bis-(2-piperidin-1-yl-ethoxy)-thioxanthen-9-one;
2,7-bis-(3-azetidin-1-yl-propoxy)-thioxanthen-9-one;
2,7-bis-(3-pyrrolidin-1-yl-propoxy)-thioxanthen-9-one;
2,7-bis-(3-piperidin-1-yl-propoxy)-thioxanthen-9-one;
2,7-bis-(2-trimethylammoniumethoxy)-thioxanthen-9-one;
2,7-bis-(3-trimethylammoniumpropoxy)-thioxanthen-9-one;
2,6-bis-(2-methylaminoethoxy)-thioxanthen-9-one;
2,6-bis-(2-ethylaminoethoxy)-thioxanthen-9-one;
2,6-bis-(2-n-propylaminoethoxy)-thioxanthen-9-one;
2,6-bis-(3-methylaminopropoxy)-thioxanthen-9-one;
2,6-bis-(3-ethylaminopropoxy)-thioxanthen-9-one;
2,6-bis-(3-n-propylaminopropoxy)-thioxanthen-9-one;
2,6-bis-(2-dimethylaminoethoxy)-thioxanthen-9-one;
2,6-bis-(2-diethylaminoethoxy)-thioxanthen-9-one;
2,6-bis-(2-di-n-propylaminoethoxy)-thioxanthen-9-one;
2,6-bis-(3-dimethylaminopropoxy)-thioxanthen-9-one;
2,6-bis-(3-diethylaminopropoxy)-thioxanthen-9-one;
2,6-bis-(3-di-n-propylaminopropoxy)-thioxanthen-9-one;
2,6-bis-(2-azetidin-1-yl-ethoxy)-thioxanthen-9-one;
2,6-bis-(2-pyrrolidin-1-yl-ethoxy)-thioxanthen-9-one;
2,6-bis-(2-piperidin-1-yl-ethoxy)-thioxanthen-9-one;
2,6-bis-(3-azetidin-1-yl-propoxy)-thioxanthen-9-one;
2,6-bis-(3-pyrrolidin-1-yl-propoxy)-thioxanthen-9-one;
2,6-bis-(3-piperidin-1-yl-propoxy)-thioxanthen-9-one;
2,6-bis-(2-trimethylammoniumethoxy)-thioxanthen-9-one;
2,6-bis-(3-trimethylammoniumpropoxy)-thioxanthen-9-one;
3,6-bis-(2-aminoethoxy)-thioxanthen-9-one;
3,6-bis-(3-aminopropoxy)-thioxanthen-9-one;
3,6-bis-(2-methylaminoethoxy)-thioxanthen-9-one;
3,6-bis-(2-ethylaminoethoxy)-thioxanthen-9-one;
3,6-bis-(2-n-propylaminoethoxy)-thioxanthen-9-one;
3,6-bis-(3-methylaminopropoxy)-thioxanthen-9-one;
3,6-bis-(3-ethylaminopropoxy)-thioxanthen-9-one;
3,6-bis-(3-n-propylaminopropoxy)-thioxanthen-9-one;
3,6-bis-(2-dimethylaminoethoxy)-thioxanthen-9-one;
3,6-bis-(2-diethylaminoethoxy)-thioxanthen-9-one;
3,6-bis-(2-di-n-propylaminoethoxy)-thioxanthen-9-one;
3,6-bis-(3-dimethylaminopropoxy)-thioxanthen-9-one;
3,6-bis-(3-diethylaminopropoxy)-thioxanthen-9-one;
3,6-bis-(3-di-n-propylaminopropoxy)-thioxanthen-9-one;
3,6-bis-(2-azetidin-1-yl-ethoxy)-thioxanthen-9-one;
3,6-bis-(2-pyrrolidin-1-yl-ethoxy)-thioxanthen-9-one;
3,6-bis-(2-piperidin-1-yl-ethoxy)-thioxanthen-9-one;
3,6-bis-(3-azetidin-1-yl-propoxy)-thioxanthen-9-one;
3,6-bis-(3-pyrrolidin-1-yl-propoxy)-thioxanthen-9-one;
3,6-bis-(3-piperidin-1-yl-propoxy)-thioxanthen-9-one;
3,6-bis-(2-trimethylammoniumethoxy)-thioxanthen-9-one;
3,6-bis-(3-trimethylammoniumpropoxy)-thioxanthen-9-one;
2,7-bis-(2-aminoethoxy)-10H-acridine-9-one;
2,7-bis-(3-aminopropoxy)-10H-acridine-9-one;
2,7-bis-(2-methylaminoethoxy)-10H-acridine-9-one;
2,7-bis-(2-ethylaminoethoxy)-10H-acridine-9-one;
2,7-bis-(2-n-propylaminoethoxy)-10H-acridine-9-one;
2,7-bis-(3-methylaminopropoxy)-10H-acridine-9-one;
2,7-bis-(3-ethylaminopropoxy)-10H-acridine-9-one;
2,7-bis-(3-n-propylaminopropoxy)-10H-acridine-9-one;
2,7-bis-(2-dimethylaminoethoxy)-10H-acridine-9-one;
2,7-bis-(2-diethylaminoethoxy)-10H-acridine-9-one;
2,7-bis-(2-di-n-propylaminoethoxy)-10H-acridine-9-one;
2,7-bis-(3-dimethylaminopropoxy)-10H-acridine-9-one;
2,7-bis-(3-diethylaminopropoxy)-10H-acridine-9-one;
2,7-bis-(3-di-n-propylaminopropoxy)-10H-acridine-9-one;
2,7-bis-(2-azetidin-1-yl-ethoxy)-10H-acridine-9-one;
2,7-bis-(2-pyrrolidin-1-yl-ethoxy)-10H-acridine-9-one;
2,7-bis-(2-piperidin-1-yl-ethoxy)-10H-acridine-9-one;
2,7-bis-(3-azetidin-1-yl-propoxy)-10H-acridine-9-one;
2,7-bis-(3-pyrrolidin-1-yl-propoxy)-10H-acridine-9-one;
2,7-bis-(3-piperidin-1-yl-propoxy)-10H-acridine-9-one;
2,7-bis-(2-trimethylammoniumethoxy)-10H-acridine-9-one;
2,7-bis-(3-trimethylammoniumpropoxy)-10H-acridine-9-one;
2,6-bis-(2-methylaminoethoxy)-10H-acridine-9-one;
2,6-bis-(2-ethylaminoethoxy)-10H-acridine-9-one;
2,6-bis-(2-n-propylaminoethoxy)-10H-acridine-9-one;
2,6-bis-(3-methylaminopropoxy)-10H-acridine-9-one;
2,6-bis-(3-ethylaminopropoxy)-10H-acridine-9-one;
2,6-bis-(3-n-propylaminopropoxy)-10H-acridine-9-one;
2,6-bis-(2-dimethylaminoethoxy)-10H-acridine-9-one;
2,6-bis-(2-diethylaminoethoxy)-10H-acridine-9-one;
2,6-bis-(2-di-n-propylaminoethoxy)-10H-acridine-9-one;
2,6-bis-(3-dimethylaminopropoxy)-10H-acridine-9-one;
2,6-bis-(3-diethylaminopropoxy)-10H-acridine-9-one;
2,6-bis-(3-di-n-propylaminopropoxy)-10H-acridine-9-one;
2,6-bis-(2-azetidin-1-yl-ethoxy)-10H-acridine-9-one;
2,6-bis-(2-pyrrolidin-1-yl-ethoxy)-10H-acridine-9-one;
2,6-bis-(2-piperidin-1-yl-ethoxy)-10H-acridine-9-one;
2,6-bis-(3-azetidin-1-yl-propoxy)-10H-acridine-9-one;
2,6-bis-(3-pyrrolidin-1-yl-propoxy)-10H-acridine-9-one;
2,6-bis-(3-piperidin-1-yl-propoxy)-10H-acridine-9-one;
2,6-bis-(2-trimethylammoniumethoxy)-10H-acridine-9-one;
2,6-bis-(3-trimethylammoniumpropoxy)-10H-acridine-9-one;
3,6-bis-(2-aminoethoxy)-10H-acridine-9-one;
3,6-bis-(3-aminopropoxy)-10H-acridine-9-one;
3,6-bis-(2-methylaminoethoxy)-10H-acridine-9-one;
3,6-bis-(2-ethylaminoethoxy)-10H-acridine-9-one;
3,6-bis-(2-n-propylaminoethoxy)-10H-acridine-9-one;
3,6-bis-(3-methylaminopropoxy)-10H-acridine-9-one;
3,6-bis-(3-ethylaminopropoxy)-10H-acridine-9-one;
3,6-bis-(3-n-propylaminopropoxy)-10H-acridine-9-one;
3,6-bis-(2-dimethylaminoethoxy)-10H-acridine-9-one;
3,6-bis-(2-diethylaminoethoxy)-10H-acridine-9-one;
3,6-bis-(2-di-n-propylaminoethoxy)-10H-acridine-9-one;
3,6-bis-(3-dimethylaminopropoxy)-10H-acridine-9-one;
3,6-bis-(3-diethylaminopropoxy)-10H-acridine-9-one;
3,6-bis-(3-di-n-propylaminopropoxy)-10H-acridine-9-one;
3,6-bis-(2-azetidin-1-yl-ethoxy)-10H-acridine-9-one;
3,6-bis-(2-pyrrolidin-1-yl-ethoxy)-10H-acridine-9-one;
3,6-bis-(2-piperidin-1-yl-ethoxy)-10H-acridine-9-one;
3,6-bis-(3-azetidin-1-yl-propoxy)-10H-acridine-9-one;
3,6-bis-(3-pyrrolidin-1-yl-propoxy)-10H-acridine-9-one;
3,6-bis-(3-piperidin-1-yl-propoxy)-10H-acridine-9-one;
3,6-bis-(2-trimethylammoniumethoxy)-10H-acridine-9-one;
3,6-bis-(3-trimethylammoniumpropoxy)-10H-acridine-9-one;
2,7-bis-(2-aminoethoxy)-10-methyl-10H-acridine-9-one;
2,7-bis-(3-aminopropoxy)-10-methyl-10H-acridine-9-one;
2,7-bis-(2-methylaminoethoxy)-10-methyl-10H-acridine-9-one;
2,7-bis-(2-ethylaminoethoxy)-10-methyl-10H-acridine-9-one;
2,7-bis-(2-n-propylaminoethoxy)-10-methyl-10H-acridine-9-one;
2,7-bis-(3-methylaminopropoxy)-10-methyl-10H-acridine-9-one;
2,7-bis-(3-ethylaminopropoxy)-10-methyl-10H-acridine-9-one;
2,7-bis-(3-n-propylaminopropoxy)-10-methyl-10H-acridine-9-one;
2,7-bis-(2-dimethylaminoethoxy)-10-methyl-10H-acridine-9-one;
2,7-bis-(2-diethylaminoethoxy)-10-methyl-10H-acridine-9-one;
2,7-bis-(2-di-n-propylaminoethoxy)-10-methyl-10H-acridine-9-one;
2,7-bis-(3-dimethylaminopropoxy)-10-methyl-10H-acridine-9-one;
2,7-bis-(3-diethylaminopropoxy)-10-methyl-10H-acridine-9-one;
2,7-bis-(3-di-n-propylaminopropoxy)-10-methyl-10H acridine-9-one;
2,7-bis-(2-azetidin-1-yl-ethoxy)-10-methyl-10H-acridine-9-one;
2,7-bis-(2-pyrrolidin-1-yl-ethoxy)-10-methyl-10H-acridine-9-one;
2,7-bis-(2-piperidin-1-yl-ethoxy)-10-methyl-10H-acridine-9-one;
2,7-bis-(3-azetidin-1-yl-propoxy)-10-methyl-10H-acridine-9-one;
2,7-bis-(3-pyrrolidin-1-yl-propoxy)-10-methyl-10H-acridine-9-one;
2,7-bis-(3-piperidin-1-yl-propoxy)-10-methyl-10H-acridine-9-one;
2,7-bis-(2-trimethylammoniumethoxy)-10-methyl-10H-acridine-9-one;
2,7-bis-(3-trimethylammoniumpropoxy)-10-methyl-10H-acridine-9-one;
2,6-bis-(2-methylaminoethoxy)-10-methyl-10H-acridine-9-one;
2,6-bis-(2-ethylaminoethoxy)-10-methyl-10H-acridine-9-one;
2,6-bis-(2-n-propylaminoethoxy)-10-methyl-10H-acridine-9-one;
2,6-bis-(3-methylaminopropoxy)-10-methyl-10H-acridine-9-one;
2,6-bis-(3-ethylaminopropoxy)-10-methyl-10H-acridine-9-one;
2,6-bis-(3-n-propylaminopropoxy)-10-methyl-10H-acridine-9-one;
2,6-bis-(2-dimethylaminoethoxy)-10-methyl-10H-acridine-9-one;
2,6-bis-(2-diethylaminoethoxy)-10-methyl-10H-acridine-9-one;
2,6-bis-(2-di-n-propylaminoethoxy)-10-methyl-10H-acridine-9-one;
2,6-bis-(3-dimethylaminopropoxy)-10-methyl-10H-acridine-9-one;
2,6-bis-(3-diethylaminopropoxy 10-methyl-10H-acridine-9-one;
2,6-bis-(3-di-n-propylaminopropoxy)-10-methyl-10-H-acridine-9-one;
2,6-bis-(2-azetidin-1-yl-ethoxy)-10-methyl-10H-acridine-9-one;
2,6-bis-(2-pyrrolidin-1-yl-ethoxy)-10-methyl-10H-acridine-9-one;
2,6-bis-(2-piperidin-1-yl-ethoxy)-10-methyl-10H-acridine-9-one;
2,6-bis-(3-azetidin-1-yl-propoxy)-10-methyl-10H-acridine-9-one;
2,6-bis-(3-pyrrolidin 1-yl-propoxy)-10-methyl-10H-acridine-9-one;
2,6-bis-(3-piperidin-1-yl-propoxy)-10-methyl-10H-acridine-9-one;
2,6-bis-(2-trimethylammoniumethoxy)-10-methyl-10H-acridine-9-one;
2,6-bis-(3-trimethylammoniumpropoxy)-10-methyl-10H-acridine-9-one;
3,6-bis-(2-aminoethoxy)-10-methyl-10H-acridine-9-one;
3,6-bis-(3-aminopropoxy)-10-methyl-10H-acridine-9-one;
3,6-bis-(2-methylaminoethoxy)-10-methyl-10H-acridine-9-one;
3,6-bis-(2-ethylaminoethoxy)-10-methyl-10H-acridine-9-one;
3,6-bis-(2-n-propylaminoethoxy)-10-methyl-10H-acridine-9-one;
3,6-bis-(3-methylaminopropoxy)-10-methyl-10H-acridine-9-one;
3,6-bis-(3-ethylaminopropoxy)-10-methyl-10H-acridine-9-one;
3,6-bis-(3-n-propylaminopropoxy)-10-methyl-10H-acridine-9-one;
3,6-bis-(2-dimethylaminoethoxy)-10-methyl-10H-acridine-9-one;
3,6-bis-(2-diethylaminoethoxy)-10-methyl-10H-acridine-9-one;
3,6-bis-(2-di-n-propylaminoethoxy)-10-methyl-10H-acridine-9-one;
3,6-bis-(3-dimethylaminopropoxy)-10-methyl-10H-acridine-9-one;
3,6-bis-(3-diethylaminopropoxy 10-methyl-10H-acridine-9-one;
3,6-bis-(3-di-n-propylaminopropoxy)-10-methyl-10-acridine-9-one;
3,6-bis-(2-azetidin-1-yl-ethoxy)-10-methyl-10H-acridine-9-one;
3,6-bis-(2-pyrrolidin-1-yl-ethoxy)-10-methyl-10H-acridine-9-one;
3,6-bis-(2-piperidin-1-yl-ethoxy)-10-methyl-10H-acridine-9-one;
3,6-bis-(3-azetidin-1-yl-propoxy)-10-methyl-10H-acridine-9-one;
3,6-bis-(3-pyrrolidin-1-yl-propoxy)-10-methyl-10H-acridine-9-one;
3,6-bis-(3-piperidin-1-yl-propoxy)-10-methyl-10H-acridine-9-one;
3,6-bis-(2-trimethylammoniumethoxy)-10-methyl-10H-acridine-9-one;
3,6-bis-(3-trimethylammoniumpropoxy)-10-methyl-10H-acridine-9-one;
3,8-bis-(2-aminoethoxy)-5H-phenanthridin-9-one;
3,8-bis-(3-aminopropoxy)-5H-phenanthridin-6-one;
3,8-bis-(2-methylaminoethoxy)-5H-phenanthridin-6-one;
3,8-bis-(2-ethylaminoethoxy)-5H-phenanthridin-6-one;
3,8-bis-(2-n-propylaminoethoxy)-5H-phenanthridin-6-one;
3,8-bis-(3-methylaminopropoxy)-5H-phenanthridin-6-one;
3,8-bis-(3-ethylaminopropoxy)-5H-phenanthridin 6-one;
3,8-bis-(3-n-propylaminopropoxy)-5H-phenanthridin-6-one;
3,8-bis-(2-dimethylaminoethoxy)-5H-phenanthridin-6-one;
3,8-bis-(2-diethylaminoethoxy)-5H-phenanthridin-6-one;
3,8-bis-(2-di-n-propylaminoethoxy)-5H-phenanthridin-6-one;
3,8-bis-(3-dimethylaminopropoxy)-5H-phenanthridin-6-one;
3,8-bis-(3-diethylaminopropoxy)-5H-phenanthridin-6-one;
3,8-bis-(3-di-n-propylaminopropoxy)-5H-phenanthridin-6-one;
3,8-bis-(2-azetidin-1-yl-ethoxy)-5H-phenanthridin-6-one;
3,8-bis-(2-pyrrolidin-1-yl-ethoxy)-5H-phenanthridin-6-one;
3,8-bis-(2-piperidin-1-yl-ethoxy)-5H-phenanthridin-6-one;
3,8-bis-(3-azetidin-1-yl-propoxy)-5H-phenanthridin-6-one;
3,8-bis-(3-pyrrolidin-1-yl-propoxy)-5H-phenanthridin-6-one;
3,8-bis-(3-piperidin-1-yl-propoxy)-5H-phenanthridin-6-one;
3,8-bis-(2-trimethylammoniumethoxy)-5H-phenanthridin-6-one;
3,8-bis-(3-trimethylammoniumpropoxy)-5H-phenanthridin-6-one;
3,8-bis-(2-aminoethoxy)-5-methyl-5H-phenanthridin-6-one;
3,8-bis-(3-aminopropoxy)-5-methyl-5H-phenanthridin-6-one;
3,8-bis-(2-methylaminoethoxy)-5-methyl-5H-phenanthridin-6-one;
3,8-bis-(2-ethylaminoethoxy)-5-methyl-5H-phenanthridin-6-one;
3,8-bis-(2-n-propylaminoethoxy)-5-methyl-5H-phenanthridin-6-one;
3,8-bis-(3-methylaminopropoxy)-5-methyl-5H-phenanthridin-6-one;
3,8-bis-(3-ethylaminopropoxy)-5-methyl-5H-phenanthridin-6-one;
3,8-bis-(3-n-propylaminopropoxy)-5-methyl-5H-phenanthridin-6-one;
3,8-bis-(2-dimethylaminoethoxy)-5-methyl-5H-phenanthridin-6-one;
3,8-bis-(2-diethylaminoethoxy)-5-methyl-5H-phenanthridin-6-one;
3,8-bis-(2-di-n-propylaminoethoxy)-5-methyl-5H-phenanthridin-6-one;
3,8-bis-(3-dimethylaminopropoxy)-5-methyl-5H-phenanthridin-6-one;
3,8-bis-(3-diethylaminopropoxy)-5-methyl-5H-phenanthridin-6-one;
3,8-bis-(3-di-n-propylaminopropoxy)-5-methyl-5H-phenanthridin-6-one;
3,8-bis-(2-azetidin-1-yl-ethoxy)-5-methyl-5H-phenanthridin-6-one;
3,8-bis-(2-pyrrolidin-1-yl-ethoxy)-5-methyl-5H-phenanthridin-6-one;
3,8-bis-(2-piperidin-1-yl-ethoxy)-5-methyl-5H-phenanthridin-6-one;
3,8-bis-(3-azetidin-1-yl-propoxy)-5-methyl-5H-phenanthridin-6-one;
3,8-bis-(3-pyrrolidin-1-yl-propoxy)-5-methyl-5H-phenanthridin-6-one;
3,8-bis-(3-piperidin-1-yl-propoxy)-5-methyl-5H-phenanthridin-6-one;
3,8-bis-(2-trimethylammoniumethoxy)-5-methyl-5H-phenanthridin-6-one;
3,8-bis-(3-trimethylammoniumpropoxy)-5-methyl-5H-phenanthridin-6-one;
3,8-bis-(2-aminoethoxy)-benzo[c]chromen-6-one;
3,8-bis-(3-aminopropoxy)-benzo[c]chromen-6-one;
3,8-bis-(2-methylaminoethoxy)-benzo[c]chromen-6-one;
3,8-bis-(2-ethylaminoethoxy)-benzo[c]chromen-6-one;
3,8-bis-(2-n-propylaminoethoxy)-benzo[c]chromen-6-one;
3,8-bis-(3-methylaminopropoxy)-benzo[c]chromen 6-one;
3,8-bis-(3-ethylaminopropoxy)-benzo[c]chromen-6-one;
3,8-bis-(3-n-propylaminopropoxy)-benzo[c]chromen-6-one;
3,8-bis-(2-dimethylaminoethoxy)-benzo[c]chromen-6-one;
3,8-bis-(2-diethylaminoethoxy)-benzo[c]chromen-6-one;
3,8-bis-(2-di-n-propylaminoethoxy)-benzo[c]chromen-6-one;
3,8-bis-(3-dimethylaminopropoxy)-benzo[c]chromen-6-one;
3,8-bis-(3-diethylaminopropoxy)-benzo[c]chromen-6-one;
3,8-bis-(3-di-n-propylaminopropoxy)-benzo[c]chromen-6-one;
3,8-bis-(2-azetidin-1-yl-ethoxy)-benzo[c]chromen-6-one;
3,8-bis-(2-pyrrolidin-1-yl-ethoxy)-benzo[c]chromen-6-one;
3,8-bis-(2-piperidin-1-yl-ethoxy)-benzo[c]chromen-6-one;
3,8-bis-(3-azetidin-1-yl-propoxy)-benzo[c]chromen-6-one;
3,8-bis-(3-pyrrolidin-1-yl-propoxy)-benzo[c]chromen-6-one;
3,8-bis-(3-piperidin-1-yl-propoxy)-benzo[c]chromen-6-one;
3,8-bis-(2-trimethylammoniumethoxy)-benzo[c]chromen-6-one;
3,8-bis-(3-trimethylammoniumpropoxy)-benzo[c]chromen-6-one;
2,7-bis-(2-aminoethoxy)-10H-phenanthren-9-one;
2,7-bis-(3-aminopropoxy)-10H-phenanthren-9-one;
2,7-bis-(2-methylaminoethoxy)-10H-phenanthren-9-one;
2,7-bis-(2-ethylaminoethoxy)-10H-phenanthren-9-one;
2,7-bis-(2-n-propylaminoethoxy)-10H-phenanthren-9-one;
2,7-bis-(3-methylaminopropoxy)-10H-phenanthren-9-one;
2,7-bis-(3-ethylaminopropoxy)-10H-phenanthren-9-one;
2,7-bis-(3-n-propylaminopropoxy)-10H-phenanthren-9-one;
2,7-bis-(2-dimethylaminoethoxy)-10H-phenanthren 9-one;
2,7-bis-(2-diethylaminoethoxy)-10H-phenanthren-9-one;
2,7-bis-(2-di-n-propylaminoethoxy)-10H-phenanthren-9-one;
2,7-bis-(3-dimethylaminopropoxy)-10H-phenanthren-9-one;
2,7-bis-(3-diethylaminopropoxy)-10H-phenanthren-9-one;
2,7-bis-(3-di-n-propylaminopropoxy)-10H-phenanthren-9-one;
2,7-bis-(2-azetidin-1-yl-ethoxy)-10H-phenanthren-9-one;
2,7-bis-(2-pyrrolidin-1-yl-ethoxy)-10H-phenanthren-9-one;
2,7-bis-(2-piperidin-1-yl-ethoxy)-10H-phenanthren-9-one;
2,7-bis-(3-azetidin-1-yl-propoxy)-10H-phenanthren-9-one;
2,7-bis-(3-pyrrolidin-1-yl-propoxy)-10H-phenanthren-9-one;
2,7-bis-(3-piperidin-1-yl-propoxy)-10H-phenanthren-9-one;
2,7-bis-(2-trimethylammoniumethoxy)-10H-phenanthren 9-one; and
2,7-bis-(3-trimethylammoniumpropoxy)-10H-phenanthren-9-one.

20. The method of selectively modulating the effects of α7nicotinic acetylcholine receptors in a mammal comprising administering an effective amount of a compound of claim 1.

21. The method according to claim 14, wherein the condition or disorder is selected from the group consisting of attention deficit disorder, attention deficit hyperactivity disorder (ADHD), Alzheimer's disease (AD), mild cognitive impairment, senile dementia, AIDS dementia, Pick's Disease, dementia associated with Lewy bodies, dementia associated with Down's syndrome, amyotrophic lateral sclerosis, Huntington's disease, diminished CNS function associated with traumatic brain injury, acute pain, post-surgical pain, chronic pain, inflammatory pain, neuropathic pain, infertility, lack of circulation, need for new blood vessel growth associate with wound healing, more particularly circulation around a vascular occlusion, need for new blood vessel growth associated with vascularization of skin grafts, ischemia, inflammation, wound healing, and other complications associated with diabetes.

22. The method according to claim 14, wherein the condition or disorder is selected from the group consisting of a memory disorder, cognitive disorder, neurodegeneration, and neurodevelopmental disorder.

23. The method according to claim 1, wherein the condition or disorder is schizophrenia.

24. The method according to claim 14, further comprising administering a compound of claim 1 in combination with an atypical antipsychotic.
